(19) United States
(12) Patent Application Publication
(10) Pub. No.: US 2024/0175022 A1
(43) Pub. Date: May 30, 2024

(54) RUNX1 Inhibition for Treatment of Proliferative Vitreoretinopathy and Conditions Associated with Epithelial to Mesenchymal Transition

(71) Applicants: The Schepens Eye Research Institute, Inc., Boston, MA (US); Massachusetts Eye and Ear Infirmary, Boston, MA (US)

(72) Inventors: Leo A. Kim, Boston, MA (US); Joseph F. Arboleda-Velasquez, Newton, MA (US); Dhanesh Amarnani, Allston, MA (US); Dean Eliott, Carlsbad, CA (US)

(21) Appl. No.: 18/351,320

(22) Filed: Jul. 12, 2023

Related U.S. Application Data
(62) Division of application No. 16/763,880, filed on May 13, 2020, now Pat. No. 11,739,326.
(60) Provisional application No. 62/586,067, filed on Nov. 14, 2017.

(51) Int. Cl. C12N 15/113; A61K 9/00; A61K 31/4025; A61K 31/5513; A61K 31/7105; A61K 45/06; A61P 27/02; G01N 33/68

(52) U.S. Cl. CPC C12N 15/113; A61K 9/0048; A61K 31/4025; A61K 31/5513; A61K 31/7105; A61K 45/06; A61P 27/02; G01N 33/6893; C12N 2310/14; G01N 2333/4706; G01N 2800/164

(57) ABSTRACT

The present subject matter provides compositions. formulations and methods for preventing or reducing proliferation or migration of retinal cells or epithelial to mesenchymal transition in ocular cells or cells from other tissues.

### RELATED APPLICATIONS

This application is a divisional application of U.S. patent application Ser. No. 16/763,880, filed May 13, 2020, which is a national stage application filed under 35 U.S.C. § 371, of International Patent Application No PCT/US2018/061110, filed on Nov. 14, 2018, which claims the benefit of priority to U.S. Provisional Application No. 62/586,067, filed Nov. 14, 2017, the entire contents of which is incorporated herein by reference.

### STATEMENT AS TO FEDERALLY SPONSORED RESEARCH

This invention was made with government support under EY021624 awarded by the National Institutes of Health and under W81XWH-17-2-0006 award by the Department of Defense. The Government has certain rights in the invention.

### SEQUENCE LISTING

This application contains a Sequence Listing that has been submitted electronically as an XML file named “00633-0305002_SL_ST26.XML.” The XML file, created on Jul. 12, 2023, is 146,433 bytes in size. The material in the XML file is hereby incorporated by reference in its entirety.

### FIELD OF THE INVENTION

The present invention relates to preventing or reducing proliferation, migration, or mesenchymal transition of retinal pigment epithelial cells.

### BACKGROUND

Retinal detachment (RD) is an important cause of sudden visual loss in the United States, with approximately 40,000 cases occurring annually. Permanent visual loss will result if treatment is delayed. A retinal detachment is the separation of the neurosensory retina from the retinal pigment epithelium (RPE). In the nonpathologic state, the retinal pigment epithelium is a continuous epithelial monolayer occluded by tight junctions, which maintain a strict separation of the underlying choroidal capillary beds from the photoreceptors of the sensory retina, thus forming the outer blood-retina barrier. Its functions include the nourishment of photoreceptors, elimination of waste products, and reabsorption of subretinal fluid.

The definitive treatment of retinal detachment is surgical repair. Multiple operative techniques are available to the treating retinologist, but the principles underlying treatment of retinal detachment remain the same: removal of fluid from the subretinal space, relief of any existing traction, and treatment and prophylaxis against the underlying cause for the ingression of fluid, whether it be due to a retinal break or an exudative process.

Proliferative vitreoretinopathy (PVR) is the most common cause for failure of retinal detachment surgery, a complication which occurs in 5-10% of all retinal detachment surgeries. PVR can also occur spontaneously in the absence of surgery. PVR is most likely to develop following repeated surgical procedures of the eye, following significant physiologic insult to the eye such as in trauma, as well as in retinal detachments complicated by multiple tears, giant tears, vitreous hemorrhage, or in eyes with uveitis. PVR is especially prevalent after retinal detachment associated with open globe injury, where it occurs in approximately 50% of cases (Colyer M., et al. Perforating globe injuries during operation Iraqi Freedom. Ophthalmology. 2008;115:2087-2093, and Eliott D. et al., Smoking is a risk factor for proliferative vitreoretinopathy after traumatic retinal detachment. Retina. 2017;37:1229-1235).

PVR is also a common complication of post-traumatic eye surgery. In this case, cells also grow uncontrollably beneath or on top of the retina triggering pre/sub-retinal membrane formation, tractional retinal detachment, and permanent vision loss. PVR occurs in 40-60% of patients with open globe injury. Hence PVR is highly relevant for the military and military-related eye trauma. Colyer, M. H., et al., Perforating globe injuries during operation Iraqi Freedom. Ophthalmology, 2008.115(11): p. 2087-93.

Currently, there are no medical treatments for PVR. Current standard of care for PVR includes invasive and complex surgery that often yields disappointing results.

### SUMMARY OF THE INVENTION

Provided herein are solutions to the clinical problems described above. The present subject matter provides methods for preventing or reducing proliferation or migration or mesenchymal transition of retinal pigment epithelial (RPE) cells or other cells including retinal glial cells, macrophages, and fibroblasts in a subject who comprises a retinal hole or a retinal tear, the method comprising administering to the subject a Runt-Related Transcription Factor 1 (RUNX1) inhibitor. For example, the methods are useful for preventing or reducing proliferation, migration, or mesenchymal transition of retinal pigment epithelial cells, corneal epithelial cells, conjunctival epithelial cells, and other cells within the eye. PVR is diagnosed by the observation of cell outgrowths, membranes and bands in the vitreous during an ophthalmological exam, fundus or optical coherence tomography (OCT). The methods and compositions described herein are also useful for RUNX1 inhibition for treatment of proliferative vitreoretinopathy and other conditions associated with epithelial to mesenchymal transition (EMT). Thus, the invention encompasses a composition for preventing or reducing proliferation or migration of retinal pigment epithelial (RPE) cells in a subject who comprises a retinal hole or retinal tear, the composition comprising a RUNX1, inhibitor, e.g., a pharmaceutical composition comprising the inhibitor and a pharmaceutically-acceptable carrier or excipient. An excipient is an inactive substance that serves as the vehicle or medium for a drug or other active substance.

In embodiments, the subject comprises proliferative vitreoretinopathy (PVR). In other aspects, the subject comprises retinal detachment, wherein the retinal detachment comprises rhegmatogenous retinal detachment, exudative detachment, or tractional retinal detachment.

In embodiments. the RUNX1 inhibitor decreases the expression and/or activity of RUNX1. The RUNX1 inhibitor, may comprise a small molecule or an inhibitory nucleic acid. In aspects, the inhibitory nucleic acid comprises an RNA interfering agent (RNAi) or an RNA expressing/encoding an inhibitory protein. In particular aspects, the RNAi comprises siRNA. In other aspects, the RUNX1 inhibitor is a small molecule. In embodiments, the small molecule comprises the structure of Formula 1:

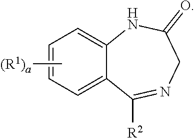

In other embodiments, the RUNX1 small molecule of Formula I comprises Ro5-3335. In embodiments, the small molecule inhibitor comprises the structure of Formula III:

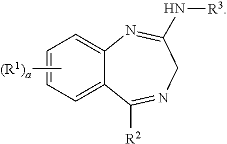

In other embodiments, the RUNX1 small molecule of Formula III comprises Ro 24-7429. In other embodiments, the RUNX1 small molecule inhibitor comprises the structure of Formula V:

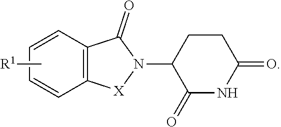

In embodiments, the small molecule of Formula V comprises lenalidomide. In other examples, the method further comprises administering methotrexate.

In embodiments, the RUNX1 inhibitor may be formulated into a composition. In other embodiments, the composition may be formulated as a solution, suspension, semi-liquid, emulsion, ointment, cream, foam gel, powder or a controlled-release/sustain-release formulation.

In aspects, the composition is administered topically or by intravitreal injection. In other aspects, the composition may be administered to the eye in a concentration of about 0.001 mg to about 10 mg of the inhibitor per eye.

In embodiments, the composition may be administered to the eye of a subject in an amount from about 50 μL to about 100 μL per eye.

In further embodiments, the composition may further comprise an anti-inflammatory agent, e.g., a combination therapy treatment approach such as one in which a RUNX1 inhibitor is administered and an anti-inflammatory agent is administered before, after, or concurrently with the anti-inflammatory agent. In embodiments, the anti-inflammatory agent comprises a steroid or a nonsteroidal anti-inflammatory drug (NSAID). For example, the anti-inflammatory agent comprises prednisolone acetate, diclofenac sodium, or a combination thereof.

In further aspects, the inhibitor may be at a concentration of about 0.001% w/v to about 100% w/v.

The subject to be treated is preferably a manual in need of such treatment, e.g., has been diagnosed with, is suffering from/has, or is at risk of developing one or more disorders or diseases described herein. The mammal can be, e.g., any mammal, e.g., a human, a primate, a mouse, a rat, a dog, a cat, a cow, a horse, or a pig. In a preferred embodiment, the mammal is a human.

In other aspects, the subject has not been diagnosed with aberrant angiogenesis. In other aspects, the subject has not been diagnosed with small vessel disease.

In aspects, the subject has not undergone surgery. In additional aspects, the inhibitor may be administered prior to a surgery, during a surgery or after a surgery. The surgery may comprise of retinal detachment surgery, glaucoma surgery, corneal surgery, cataract surgery, or any penetrating surgery of the eye. In other aspects, the subject has suffered a trauma to the eye.

Also provided herein are methods for diagnosing aberrant epithelial to mesenchymal transition (EMT) of retinal and ocular cells in a subject, the methods comprising: providing a test sample from said subject (e.g., via ocular surgery), assaying the level of runt-related transcription factor 1 (RUNX1) protein or mRNA in the test sample; and, and thereby diagnosing the subject as having aberrant EMT of retinal cells if the level of RUNX1 protein or mRNA is elevated in the test sample compared to a normal control. In embodiments, diagnosis of aberrant epithelial to mesenchymal transition of retinal cells may be performed by clinical exam or imaging. Such methods may also be used to measure RUNX1 levels, e.g., level of expression, as a biomarker for PVR and/or an indication of the severity of the disorder. The methods are also useful to monitor the effect/efficacy of treatment.

In embodiments, levels of RUNX1 may be measured using ELISA, Q-RT-PCR, Western blot analysis, immunohistochemistry, or immunofluorescence. In other embodiments, the test sample from the subject may be obtained during ocular surgery.

Also provided herein are methods for treating or reducing the severity of proliferative vitreoretinopathy (PVR) in a subject, the method comprising: identifying a subject comprising PVR, and administering to said subject a runt-related transcription factor 1 (RUNX1) inhibitor.

Each embodiment disclosed herein is contemplated as being applicable to each of the other disclosed embodiments. Thus, all combinations of the various elements described herein are within the scope of the invention.

Also described herein are methods for monitoring whether a disease (e.g., that comprises proliferative vitreoretinopathy (PVR) as well as a number of other disorders described herein, e.g., pathologic ocular fibrosis, or pathologic ocular proliferation, is progressing in a subject who has been diagnosed with the disease. The method comprises periodically determining the level of RUNX1 protein or mRNA in said subject, and identifying the disease as worsening if the level of RUNX1 protein or mRNA increases over time: identifying the disease as improving if the level of RUNX1 protein or mRNA decreases over time; identifying the disease as neither worsening or improving if the level of RUNX1 protein or mRNA remains the same or about the same over time, wherein determining the level of RUNX1 protein or mRNA comprises providing a test sample from said subject; and assaying the level of RUNX1 protein or mRNA in the test sample.

Described herein is a method for diagnosing a proliferative vitreoretinopathy (PVR) in a subject, the method comprising providing a test sample from said subject, assaying the level of runt-related transcription factor 1 (RUNX1) protein or mRNA in the test sample; and diagnosing the subject as having aberrant PVR if the level of RUNX1 protein or mRNA is elevated in the test sample compared to a normal control.

Also provided herein are methods for identifying whether a therapy has reduced or ameliorated a disease that comprises proliferative vitreoretinopathy (PVR) in a subject, the method comprising providing a pre-therapy test sample from said subject, assaying the pre-therapy level of RUNX1 protein or mRNA in the pre-therapy test sample; administering the therapy to the subject; providing a post-therapy test sample from said subject; assaying the post-therapy level of RUNX1 protein or mRNA in the post-therapy test sample; and identifying the therapy as having reduced or ameliorated said disease if the level of RUNX1 protein or mRNA in the post-therapy test sample is lower than the level of RUNX1 protein or mRNA in the pre-therapy sample.

In other examples, described herein are methods of reducing proliferation and migration of cells undergoing epithelial to mesenchymal transition (EMT) within an eye of a subject, the method comprising administering a runt-related transcription factor 1 (RUNX1) inhibitor to the subject. In embodiments, the EMT related disease comprises pathologic ocular fibrosis, proliferation, conjunctival fibrosis, ocular cicatricial pemphigoid, corneal scarring, corneal epithelial down growth, or aberrant post-surgical fibrosis. In some examples, the inhibitor is administered during or after glaucoma surgery, cataract surgery, or Laser-Assisted in situ keratomileusis (LASIK). In other examples, the inhibitor is administered during or after intraocular surgery.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### DESCRIPTION OF THE DRAWINGS

FIG. 1A is an image depicting the immunofluorescence staining of a negative control counterstained with DAPI. FIG. 1B is an image depicting the immunofluorescence staining of a negative control of staining of RUNX1. FIG. 1C is a merged image depicting the immunofluorescence staining of FIG. 1A and FIG. 1B. FIG. 1D is an image depicting the immunofluorescence staining of the RUNX1 primary antibody counterstained with DAPI. FIG. 1E is an image depicting the immunofluorescence staining of the RUNX1. FIG. 1F is a merged image depicting the immunofluorescence staining of FIG. 1D and 1E.

FIG. 2A is an image depicting the immunofluorescence staining of patient sample 5 (membranes obtained from patient with PVR), counterstained with DAPI. FIG. 2B is an image depicting the immunofluorescence staining of RUNX1 in patient sample 5. FIG. 2C is a merged image of FIG. 2A and 2B. FIG. 2D is an image depicting the immunofluorescence staining of patient sample 3, counterstained with DAPI. FIG. 2E is an image depicting the immunofluorescence staining of RUNX1 in patient sample 3. FIG. 2F is a merged image of FIG. 2D and 2E. FIG. 20 is an image depicting the immunofluorescence staining of patient sample 11 (membranes obtained from patient with PVR) counterstained with DAPI. FIG. 2H is an image depicting he immunofluorescence staining of RUNX1 in patient sample 11. FIG. 2I is a merged image of FIG. 2G and 2H. FIG. 2A-2I indicated that RUNX1 was present in Cells from human PVR (C-PVR) from several patient donors.

FIG. 3A is an image depicting the immunofluorescence staining of RUNX1 in human PVR membranes (e.g., patients with PVR). FIG. 3B is an image depicting the immunofluorescence staining of RUNX1 in human PVR membranes counterstained with DAPI. FIG. 3C is an image depicting the immunofluorescence staining of a negative control counterstained with DAPI. FIG. 3D is an image depicting the immunofluorescence staining of a negative control counterstained with DAPI.

FIG. 4A is an image depicting the immunofluorescence staining of membranes from patients with PVR counterstained with DAPI. FIB. 4B is an image depicting the immunofluorescence staining of membranes from patients with PVR stained with RUNX1. FIG. 4C is a merged image of FIG. 4A and 4B. FIG. 4D is an image depicting the immunofluorescence staining of membranes from patients with PVR counterstained with DAPI. FIG. 4E is an image depicting the immunofluorescence staining of membranes from patients with PVR stained with RUNX1. FIG. 4F is a merged image of FIG. 4D and 4E. FIG. 4A-4F showed additional images of staining of RUNX1 in PVR.

FIG. 5A is an image depicting the immunohistochemical staining of human PVR membranes of a control, counterstained with hematoxylin in membranes obtained from patients with PVR. FIG. 5B is an image depicting the immunohistochemical staining of human PVR membranes of RUNX1, counterstained with hematoxylin in membranes obtained from patients with PVR.

FIG. 6 is a bar graph depicting that siRNA effectively knocked down expression of RUNX1 in C-PVR cells. A significant reduction was observed in the gene expression of RUNX1 in C-PVR cells 48 hours after transfection with siRUNX1compared to siScramble.

FIG. 7A is an image depicting DAPI staining of untreated controls. FIG. 7B is an image depicting RUNX1 staining of untreated controls. FIG. 7C is a merged image of FIG. 7A and 7B. FIG. 7D is an image depicting DAPI staining of siScramble. FIG. 7E is an image depicting RUNX1 staining of siScramble. FIG. 7F is a merged image of FIG. 7E and 7F. FIG. 7G is an image depicting DAPI staining of siRUNX1. FIG. 7H is an image depicting RUNX1 staining of siRUNX1. FIG. 7I is a merged image of FIG. 7G and 7H. Ki67 staining 48 hours post siRNA knockdown of RUNX1 (FIG. 7G-7I) compared to scramble (FIG. 7D-7F) and untreated controls (FIG. 7A-7C) showed a significant reduction in cell number and proliferative capacity of C-PVR cells.

FIG. 8 is a bar graph depicting the quantification of proliferation from siRUNX1, siScramble and untreated cells. A significant reduction was observed in the number of Ki67 positive proliferating cells 48 hours after transfection with siRUNX1 compared to siScramble and untreated cells. The graph depicts quantification of images from FIG. 7A-7F.

FIG. 9A is an image depicting a control (vehicle treated) Ki67 staining 48 hours post treatment. FIG. 9B is an image depicting Ki67 staining 48 hours post treatment with RUNX1 inhibitor, Ro5-3335 at 150 μM. A significant reduction in cell number and proliferative capacity of C-PVR cells was observed. FIG. 9C is a bar graph depicting the quantitation of images from FIG. 9A and 9B.

FIG. 10A is an image depicting DAPI staining of untreated controls. FIG. 10B is an image depicting Ki67 staining of untreated controls. FIG. 10C is a merged image of FIG. 10A and 10B. FIG. 10D is an image depicting DAPI staining of cells treated with RUNX1 inhibitor, Ro5-3335. FIG. 10E is an image depicting Ki67 staining of cells treated with RUNX1 inhibitor, Ro5-3335. FIG. 10F is a merged image of FIG. 10D and 10E. FIG. 10A-10E are a higher magnification than FIG. 9A and 9B; and depict a significant reduction in the cell number and proliferative capacity of C-PVR cells.

FIG. 11A is a bright-field image depicting ARPE-19) cells 7 days post-treatment of control cells. FIG. 11B is a bright-field image depicting ARPE-19 cells 7 days post-treatment with TGFβ2, TNFα, and IL-6 (from Preprotec). FIG. 11A and 11B showed epithelial-mesenchymal transition (FIG. 11B) compared to control (FIG. 11A).

FIG. 12 are inmunofluorescence staining images of ARPE-19 cells 7 days post treatment with TGFβ1, TGFβ2, and combination of TGFβ2, TNFα and IL-6 (10 ng/ml each). A reduction in the epithelial marker (cytokeratin-middle panel) was observed, and increase in the mesenchymal marker (smooth muscle actin-right panel) in the treatments was compared as compared to controls. This showed that ARPE-19 cells underwent EMT with combination treatment.

FIG. 13 are immunofluorescence staining images of mature ARPE-19 cells 7 days post treatment with TGFβ1, TGFβ2, and combination of TGFβ2, TNFα and IL-6 (10 ng/ml). A reduction in the epithelial markers, cytokeratin (middle panel) and Zonula occludens-1 (ZO-1) (right panel) in the treatments was observed as compared to controls. Losing ZO-1 organization is a marker of EMT. Therefore data showed that combination treatment also impacted ZO-1 distribution.

FIG. 14A is an immunofluorescence image depicting mature ARPE-19 cells 7 days post treatment of untreated control. FIG. 14B is an immunofluorescence image depicting mature ARPE-19 cells 7 days post treatment with TGFBI. FIG. 14C is an immunofluorescence image depicting mature ARPE-19 cells 7 days post treatment with TGFβ2. FIG. 14D is an immunofluorescence image depicting mature ARPE-19 cells 7 days post treatment with a combination of TGF822, TNFα, and IL-6 (10 ng/ml each). A reduction in the epithelial markers Zonula oceludens-1 in the treatments was observed as compared to controls.

FIG. 15 are immunofluorescence staining images of ARPE-19 cells 7 days post treatment with TGFβ1, TGFβ2, and combination of TGFβ2, TNFα and IL-6 (10 ng/ml each). A significant increase in the RUNX1 expression in the treatments was observed as compared to controls. This indicated that RUNX1 expression increased with EMT.

FIG. 16 are immunofluorescence staining of ARPE-19 cells 7 days post treatment with a combination of TGFβ2, TNFα and IL-6 (10 ng/ml each). A significant increase in the RUNX1 expression in the treatments compared to controls was observed. FIG. 16 is magnified data from FIG. 13. FIG. 17 are immmofluorescence staining images of ARPE-19 cells 7 days post treatment with a combination of TGFβ2, TNFα and IL-6 (10 ng/ml each). A significant increase in the RUNX1 expression (middle panel) and a reduction in the epithelial marker cytokeratin (merge-right panel) in the treatments was observed compared to controls. This showed that RUNX1 increased expression was also present at the 3-week time point when ZO-1 changes of distribution were present.

FIG. 18A is an image depicting a primary explant culture treated with a control (4× magnification). FIG. 18B is an image depicting a primary explant culture treated with RUNX1 inhibitor, Ro5-3335 at 150 μM (4× magnification). FIG. 18C is an image depicting a primary explant culture treated with a control (10× magnification). FIG. 18D is an image depicting a primary explant culture treated with RUNX1 inhibitor, Ro5-3335 (10× magnification). Growth was observed from control explants compared to the explants treated with RUNX1 inhibitor, which showed no growth after 4 days.

FIG. 19A are immunofluorescence staining images of RUNX1 and counterstained with DAPI in membranes obtained from patients with PVR (Case 1 “PVR 01” and Case 3 “PVR 03”). These images showed that RUNX1 expression was a common feature in membranes from different donors with PVR. Scale bars-400 microns.

FIG. 19B are immunofluorescence staining images of RUNX1 and counterstained with DAPI in membranes obtained from patients with PVR (Case 1 and Case 3). These images showed that RUNX1 expression was a common feature in membranes from different donors with PVR. Scale bars- 400 microns.

FIG. 20A are immunofluorescence staining images of Ki67 (also known ask antigen Ki-67 or Ki-67 or MKI67) (bottom, middle, FIG. 20B), RUNX1 (top, middle, FIG. 20A) and counterstained with DAPI in contiguous sections of membranes obtained from patients with PVR. These images showed that the population of cells expressing RUNX1 within PVR membranes actively proliferated, as demonstrated by their expression of Ki67. Scale bars-400 microns.

FIG. 20B are immunofluorescence staining images of Ki67 (bottom, middle, FIG. 20B), RUNX1 (top, middle, FIG. 20A) and counterstained with DAPI in contiguous sections of membranes obtained from patients with PVR. These images showed that the population of cells expressing RUNX1 within PVR membranes actively proliferated, as demonstrated by their expression of Ki67. Scale bars-400 microns.

FIG. 21A are images depicting immunoblots indicating that RUNX1 protein expression levels were increased in growth factor induced Epithelial to Mesenchymal Transition (EMT). The increase in RUNX1 protein was observed upon treatment with growth factors: transforming growth factor beta 2 (TGFβ2), tumor necrosis factor alpha (TNFα) and combination treatment, including TGFβ2, TNFα, and interleukin-6 (IL-6), at 3 and 7 days post treatment.

FIG. 21B are bar graphs indicating that an increase in RUNX1 and runt related transcription factor 2 (RUNX2) RNA expression was observed upon treatment with growth factors (TGFβ2, TNFα, and IL-6, each at 10 ng/ml) at 3 and 7 days post treatment. (“Con” depicts control). *P<0.05. **P<0.01, ****P<0.0001.

FIG. 22 are bright field images depicting ARPE-19 cells 3 days post treatment with TGFβ2, TNFα, and a combination of TGFβ2, TNFα and IL-6 with RUNX1 inhibitor (Ro5-3335). 150 μM of Ro5-3335 showed either no or reduced EMT (right panels), compared to vehicle treatment (left panels). EMT was determined by morphological changes including cell shape. This data demonstrated that RUNX1 inhibition using Ro5-3335 resulted in inhibition of EMT. Scale bars-400 microns.

FIG. 23A are immunofluorescence staining images of ARPE-19 cells 3 days post treatment with TGFβ2, TNFα, and combination of TGFβ2, TNFα and IL-6 (10 ng/ml each). Also shown is treatment without a RUNX1 small molecule inhibitor (Ro5-3335), assessed as a reduction in the epithelial marker (cytokeratin-right panel) and an increase in the mesenchymal markers such as fibronectin (middle panel) and smooth muscle actin (left panel). Scale bars—200 microns.

FIG. 23B are immunofluorescence staining images of ARPE-19 cells 3 days post treatment with TGFβ2, TNFα, and combination of TGFβ2, TNFα and IL-6 (10 ng/mL each). The data showed that treatment with a RUNX1 small molecule inhibitor (Ro5-3335) prevented EMT, which was assessed as a reduction in the epithelial marker (cytokeratin—right panel) and an increase in the mesenchymal markers such as fibronectin (middle panel) and smooth muscle actin (left panel). Scale bars—200 microns.

FIG. 24 are bright field images of C-PVR cells 7 days post treatment with TGFβ2, TNFα, and a combination of TGFβ2, TNFα and IL-6, which showed EMT compared to control (top, left picture). EMT was assessed as morphological changes including cell shape with more elongated, fibroblast-like shaped cells in the combination treatment. This experiment showed that TNFα, TGFβ2 and IL-6 induced EMT in C-PVR cells derived from human proliferative vitreoretinopathy membranes. Scale bars—400 microns.

FIG. 25 are immunofluorescence staining images of C-PVR cells 7 days post treatment with TGFβ2, TNFα and a combination of TGFβ2, TNFα and IL-6 (10 ng/ml each), which showed a reduction in the epithelial marker (cytokeratin—middle panel) and increase in the mesenchymal marker (smooth muscle actin—right panel) in the treatments compared to controls (top row). This experiment demonstrated that these growth factors induced EMT in C-PVR cells from human proliferative vitreoretinopathy. Scale bar—200 microns.

FIG. 26A is a bar graph depicting that RUNX1 expression levels were increased in growth factor-induced EMT. An increase in RUNX1 RNA was observed along with an increase in mesenchymal markers, N-Cadherin, Smooth muscle actin and vimentin and a decrease in Epithelial marker, occludin was on treatment with growth factors TGFβ2, TNFα and combination treatment of TGFβ2, TNFα and IL-6 at 7 days post treatment.

FIG. 26B are images of immunoblots showing that an increase in the protein for RUNX1, N-Cadherin (Mesenchymal marker), Snail, and Twist (Snail and Twist are EMT transition markers) was observed compared to β-actin, which was used as a loading control on treatment with growth factors at 7 days post treatment. ****P<0.0001.

FIG. 27A is a bar graph showing results of a CyQuant cell proliferation assay 24 hours post treatment with a RUNX1 inhibitor (Ro5-3335) at 100 μM, 50 μM and 25 μM, and methotrexate at 400 μM, 100 μM and 10 μM, alone or in combination compared to vehicle treated. The data showed a significant reduction in percent live cells at 24 hours. This experiment showed that RUNX1 inhibition was more efficacious than treatment with methotrexate at inhibiting growth of ARPE-19 cells. The data also showed that RUNX1 inhibition can be used as an adjuvant in combination to treatments like methotrexate.*P<0.05. **P<0.01, ***P<0.001, ****P<0.0001.

FIG. 27B is a bar graph showing results of a CyQuant cell proliferation assay 48 hours post treatment with a RUNX1 inhibitor (Ro5-3335) at 100 μM, 50 μM and 25 μM, and methotrexate at 400 μM, 100 μM and 10 μM, alone or in combination compared to vehicle treated. The data showed a significant reduction in percent live cells at 48 hours. This experiment showed that RUNX1 inhibition was more efficacious than treatment with methotrexate at inhibiting growth of ARPE-19 cells. The data also showed that RUNX1 inhibition can be used as an adjuvant in combination to treatments like methotrexate. *P<0.05, **P<0.01, ***P<0.001, ****P<0.0001.

FIG. 28A is a bar graph showing results of a cell proliferation assay at 24 hours post treatment with a RUNX1 inhibitor (Ro5-3335) at 100 μM, 50 μM and 25 μM, and methotrexate at 400 μM, 100 μM and 10 μM, alone or in combination compared to vehicle treated. The data showed a significant reduction in percent live cells at 24 hours. This experiment showed that RUNX1 inhibition was more efficacious than treatment with methotrexate at inhibiting growth of C-PVR cells. The data also showed that RUNX1 inhibition can be used as an adjuvant in combination to treatments like methotrexate. *P<0.05, **P<0.01, ***P<0.001, ****P<0.0001.

FIG. 28B is a bar graph showing results of a cell proliferation assay at 48 hours post treatment with a RUNX1 inhibitor (Ro5-3335) at 100 μM, 50 μM and 25 μM, and methotrexate at 400 μM, 100 μM and 10 μM, alone or in combination compared to vehicle treated. The data showed a significant reduction in percent live cells at 48 hours. This experiment showed that RUNX1 inhibition was more efficacious than treatment with methotrexate at inhibiting growth of C-PVR cells. The data also showed that RUNX1 inhibition can be used as an adjuvant in combination to treatments like methotrexate. *P<0.05, **P<0.01, ***P<0.001, ****P<0.0001.

FIG. 29 are bright field images of ARPE-19 cells. RUNX1 knockdown using siRNA was effective at inhibiting EMT in cells treated with a growth factor cocktail including TGF822, TNFα and IL-6, compared to untransfected (right top) and si-scramble (right middle). EMT was determined by morphological changes including cell shape. This data demonstrated that RUNX1 inhibition using siRNA resulted in inhibition of EMT. Scale bars-400 microns.

FIG. 30A is an image of an immunoblot showing RUNX1 protein levels in ARPE-19 cells treated with TGFβ2 and TNFα. These data showed that each of these growth factors induced protein expression of RUNX1. The data also showed that siRNA treatment efficiently reduced the induction of RUNX1 triggered by each of these growth factors alone. These data demonstrated that RUNX1 inhibition can be used to limit the effect of these growth factors as it relates to PVR.

FIG. 30B is an image of an immunoblot showing RUNX1 protein levels in ARPE-19 cells treated with IL-6 or with a combination of these growth factors (combination comprises TGFβ2, TNFα, and IL-6). These data showed that each of these growth factors induced protein expression of RUNX1 and that such induction was stronger when the growth factors were combined. It also showed that siRNA treatment efficient reduced the induction of RUNX1 triggered by each of these growth factors alone or in combination. These data demonstrated that RUNX1 inhibition can be used to limit the effect of these growths factors as it relates to PVR.

FIG. 31 is a series of bright field images of C-PVR cells, 72 hours after RUNX1 knockdown and 48 hours post treatment with a combination of TGFβ2, TNFα and IL-6, and which showed no or reduced EMT (right bottom picture) compared to un-transfected (right top) and Scramble (right middle). EMT was determined by morphological changes including cell shape. These data demonstrate that RUNX1 inhibition using siRNA resulted in inhibition of EMT. Scale bars—1000 microns.

FIG. 32A is a series of images indicating that apoptosis, less branching and a faster regression of the branches was observed in human PVR explants treated with RUNX1 inhibitor, Ro5-3335 at 150 μm. Detachment of the branches from the primary tissue was also observed. Top panels showed explant treated with DMSO (vehicle) and bottom panels showed explant after treatment with Ro5-3335.

FIG. 32B is a bar graph indicating the number of outgrowths (or branches) after 24 hours and 72 hours with the control (DMSO) and with Ro5-3335.

FIG. 33A is a series of images indicating that apoptosis, less branching and a faster regression of the branches was observed in another human PVR explant treated with RUNX1 inhibitor, Ro5-3335 at 150 μm. Detachment of the branches from the primary tissue was also observed. Top panels showed explant treated with DMSO (vehicle) and bottom panels showed explant after treatment with Ro5-3335.

FIG. 33B is a bar graph indicating the number of outgrowths (or branches) after 24 hours and 72 hours with the control (DMSO) and with Ro5-3335.

FIG. 34A is a series of images indicating that the RUNX1 inhibitor. Ro5-3335 reduced growth of human PVR membranes in an explant model. A synergistic effect was observed with methotrexate+Ro5-3335. Less branching, a faster regression of the branches and apoptosis was observed in human PVR explants treated with RUNX1 inhibitor (Ro5-3335 at 150 μM), or a combination of Ro5-3335 (150 μM) and methotrexate (400 μM). Detachment of the branches from the primary tissue was also observed in Ro-3335 alone, but the combination (synergistic) effect of the drugs was surprising and even more significant. Top panels showed explant treated with DMSO (vehicle), middle panels showed explant after treatment with Ro5-3335 and bottom panels showed the effect of combination treatment (Ro5-3335 and methotrexate).

FIG. 34B is a bar graph indicating the number of outgrowths (or branches) after 24 hours and 72 hours with the control (DMSO), Ro5-3335, and combination of methotrexate and Ro5-3335.

FIG. 35A is a bar graph indicating that at 40 nM and 72 hours lenalidomide inhibited proliferation in C-PVR cells. The data were compared to vehicle treated, and showed a significant reduction in percent live cells at 72 hours. *P<0.05.

FIG. 35B is a bar graph indicating that at 80 nM and 72 hours, lenalidomide inhibited proliferation in C-PVR cells. The data were compared to vehicle treated, and showed a significant reduction in percent live cells at 72 hours. *P<0.05.

FIG. 35C is a bar graph indicating that at 40 nM and 48 hours, lenalidomide inhibited proliferation in C-PVR cells. *P<0.05.

FIG. 35D is a bar graph indicating that at 80 nM and 48 hours, lenalidomide inhibited proliferation in C-PVR cells. *p<0.05.

### DETAILED DESCRIPTION

The present disclosure relates to methods for treating retinal detachment disorders, (e.g., proliferative vitreoretinopathy, PVR). Currently there are no medical (e.g., no non-surgical) treatments or management for PVR, which is a severe complication of retinal detachment, and commonly associated with eye trauma. Current management of PVR includes a potentially risky and invasive surgical method to remove the membrane directly from the eye. The compositions and methods are useful for the treatment of diseases afflicted with proliferation or migration of retinal pigment epithelial cells, glial, inflammatory cells, and mesenchymal cells as found in PVR.

The disclosure described herein is based on the surprising discovery that the Runt-Related Transcription Factor 1 (RUNX1) is highly expressed in surgically removed human PVR membranes, and cells derived from them (C-PVR). RUNX1 has been implicated in other biological processes including endothelial-cell derived blood vessel formation during embryonic development and normal angiogenesis. However, a role for RUNX1 in PVR (or EMT in retinal cells) has not previously been described. The disclosure herein describes actual patient-derived PVR membranes as a resource to highlight the specific expression of RUNX1 and to study its effect on RUNX1 inhibition. Current methods rely on surrogate cell types thought to be involved in the pathogenesis of PVR.

Additionally, RUNX1 expression is enhanced in an in vitro model of retinal pigment epithelial (RPE) cells undergoing epithelial to mesenchymal transition (EMT). The inhibition or RUNX1 via siRNA or a small molecule inhibitor significantly reduced the growth and migration of C-PVR culture. RUNX1 inhibition also significantly reduced the growth of human PVR membrane explants in culture.

Additionally, RUNX1 inhibition significantly reduced EMT in two in vitro models using ARPE-19 and C-PVR cultures. Reduction of EMT was characterized by morphological changes, increased expression in epithelial markers, and decreased expression of mesenchymal markers.

Accordingly, provided herein, are methods for preventing and treating PVR and other conditions associated with EMT by targeting RUNX1. This will allow for specific medical management of membranes, and caused their regression and impeded their growth and thus potentially avoiding the need for surgery.

### Retinal Detachment Disorder

Retinal detachment is a disorder of the eye in which the neurosensory retina separates from the retinal pigment epithelial layer underneath. The mechanism most commonly involves a break in the retina that then allows the fluid in the eye to get behind the retina. A break in the retina can occur from a posterior vitreous detachment, injury to the eye, or inflammation of the eye. Other risk factors include being short sighted and previous cataract surgery. Typically, diagnosis is accomplished by either looking at the back of the eye with an ophthalmoscope or by ultrasound.

Symptoms include an increase in the number of floaters, flashes of light, and worsening of the outer part of the visual field, which may be described as a curtain over part of the field of vision. In about 7% of cases both eyes are affected. Without treatment permanent loss of vision may occur. In patients with a retinal tear, efforts to prevent it becoming a detachment include cryotherapy using a cold probe or photocoagulation using a laser. Treatment of retinal detachment should be carried out in a timely manner.

Retinal detachment can be examined by fundus photography or ophthalmoscopy. Ultrasound has diagnostic accuracy similar to that of examination by an ophthalmologist. Recent meta-analysis shows the diagnostic accuracy of emergency department (ED) ocular ultrasonography is high. The sensitivity and specificity ranged from 97% to 100% and 83% to 100%. The typical feature of retinal detachment when viewed on ultrasound is “flying angel sign,” it shows the detached retina moving with a fixed point under the B mode, linear probe 10 MHZ. In embodiments, retinal detachment may be visualized by a fundus exam. Alternatively, a B-scan or wide-field fundus photography may be used to visualize retinal detachment.

Retinal detachments affect between 0.6 and 1.8 people per 10,000 per year. About 0.3% of people are affected at some point in their life. It is most common in people who are in their 60s or 70s, and males are more often affected than females. The long term outcomes depend on the duration of the detachment and whether the macula was detached. If treated before the macula detaches outcomes are generally good. Optionally, the subject has not been diagnosed or characterized with some other ocular disorder comprising age-related macular degeneration or an ocular angiogenesis disease or disorder.

When the retina is pulled away from the back of the eye, it is a retinal detachment. Typically, the vitreous moves away from the retina without causing problems. But sometimes the vitreous pulls hard enough to tear the retina in one or more places, and thus causing a retinal tear. Fluid may pass through a retinal tear, lifting the retina off the back of the eye. The symptoms of vitreous separation, retinal tear, and retinal detachment are similar and sometimes can overlap. On occasion, the patient may notice the floaters and flashing lights (photopsia) more commonly associated with isolated vitreous separation. An ophthalmologist, optometrist, or primary care physician may be suspicious about a more serious problem if symptoms are of very recent or sudden onset and are accompanied by a shower of spots or “cobwebs.” Of even greater concern is the loss of peripheral vision, which may present as a shadow moving toward the center of one's field of vision.

Additionally, in retinal detachment, a retinal hole may develop. Because the vitreous is attached to the retina with tiny strands of collagen, it can pull on the retina as it shrinks. Sometimes, this shrinkage can tear off a small piece of the retina in the periphery, causing a hole or tear of the periphery retina. If this missing piece of retina is in the macula, it's called a macular hole. Additionally, another direct cause of macular holes due to vitreous shrinkage is when the collagen strands stay attached to the retina forming an epiretinal membrane. These membranes can contract around the macula, causing the macula to develop a hole from the traction. Retinal detachments commonly occur secondary to peripheral retinal tears/holes, and rarely form macular holes.

In other aspects, a minority of retinal detachments result from trauma, including blunt blows to the orbit, penetrating trauma, and concussions to the head.

There are three types of retinal detachment:
(1) Rhegmatogenous retinal detachment—A rhegmatogenous retinal detachment occurs due to a break in the retina (e.g., a retinal tear) that allows fluid to pass from the vitreous space into the subretinal space between the sensory retina and the retinal pigment epithelium. Retinal breaks are divided into three types—holes, tears and dialyses. Holes form due to retinal atrophy especially within an area of lattice degeneration. Tears are due to vitreoretinal traction. Dialyses are very peripheral and circumferential, and may be either tractional or atrophic. The atrophic form most often occurs as idiopathic dialysis of the young.
(2) Exudative serous, or secondary retinal detachment—An exudative retinal detachment occurs due to inflammation, injury or vascular abnormalities that results in fluid accumulating underneath the retina without the presence of a hole, tear, or break. In evaluation of retinal detachment it is critical to exclude exudative detachment as surgery will make the situation worse, not better. Although rare, exudative detachment can be caused by the growth of a tumor on the layers of tissue beneath the retina, namely the choroid. This cancer is called a choroidal melanoma.
(3) Tractional retinal detachment—A tractional retinal detachment occurs when fibrous (from PVR membrane) or fibrovascular (from neovascular disorders such as proliferative diabetic retinopathy) tissue, caused by an injury, inflammation or neovascularization, pulls the sensory retina from the retinal pigment epithelium

### Proliferative Vitreoretinopathy

Proliferative vitreoretinopathy (PVR) is a clinical syndrome that develops as a complication of rhegmatogenous retinal detachment and is also commonly associated with eye trauma. PVR is the most common cause of failure in retinal detachment surgery, however, it can also occur with untreated eyes with retinal detachment. In particular, PVR can occur with vitreous hemorrhage, after cryotherapy, after laser retinopexy, pneumatic retinopexy, scleral buckling, or vitrectomy, and after a variety of surgical complications. PVR is also common after eye traumas (e.g., penetrating injuries) and other conditions associated with prolonged inflammation.

PVR occurs in about 8-10% of patients undergoing primary retinal detachment surgery and prevents the successful surgical repair of rhegmatogenous retinal detachment. PVR can be treated with surgery to reattach the retina, however, the visual outcome of the surgery is very poor. If PVR is progressive, then despite complex surgery, low vision in the eye results.

Pathophysiology

PVR is characterized by proliferation or migration of cells derived from retinal pigment epithelium (RPE), glia, or inflammatory recruitment on the retinal surface and within the vitreous gel. These cells transdifferentiate and take on contractile properties. The process of PVR can start when there is an interruption to the surface lining (e.g., through posterior vitreous detachment and local preretinal membrane formation or retinal tears in the periphery). The PVR process is self-propagating and is often considered an inappropriate excess wound-healing response. The cellular proliferation can increase the influx of inflammatory cytokines and inflammatory cells.

In embodiments, as described herein proliferation or migration of RPE cells describes their transdifferentiation to assume contractile properties through internal cellular contractile proteins and by laying down extracellular collagen. The cells can multiply and grow along any available scaffolding (e.g., the retinal surfaces or elements of the residual vitreous gel). The mass contraction can lead to retinal wrinkles, folds, tears, and traction retinal detachment.

During rhegmatogenous retinal detachment, fluid from the vitreous humor enters a retinal hole. The accumulation of fluid in the subretinal space and the tractional force of the vitreous on the retina result in rhegmatogenous retinal detachment. During this process the retinal cell layers come in contact with vitreous cytokines. These cytokines trigger the ability of the retinal pigmented epithelium (RPE) to proliferate and migrate. The process involved resembles fibrotic wound healing by the RPE cells. The RPE cells undergo epithelial-mesenchymal transition (EMT) and develop the ability to migrate out into the vitreous. During this process the RPE cell layer-neural retinal adhesion and RPE-ECM (extracellular matrix) adhesions are lost. The RPE cells lay down fibrotic membranes while they migrate and these membranes contract and pull at the retina. Thus, this leads to secondary retinal detachment after primary retinal detachment surgery.

During RPE disruption, inflammation may play an important role in the development of PVR. Cytokines IL-6, IL-1, TNF-α and IFN-γ have been identified in high concentrations in the vitreous in the early, proliferative stages of PVR, but they decrease to normal levels in the scarring phase. Other molecules involved in PVR include TGFβ and IL-6.

Risk Factors and Clinical Signs

As described above, the most common development of PVR is after a retinal detachment surgery and/or repair, although patients can develop PVR spontaneously with retinal detachment prior to surgery or with longstanding primary detachments. Multiple factors have been associated with the formation of PVR. In general, processes that increase vascular permeability are more likely to increase the probability of PVR formation. Specific risk factors that have been identified include: uveitis; large, giant, or multiple tears; vitreous hemorrhage, preoperative or postoperative choroidal detachments; aphakia; multiple previous surgeries; and large detachments involving greater than 2 quadrants of the eye.

Early signs of PVR are often subtle and can include cellular dispersion in the vitreous and on the retinal surface, which can appear as a white opacification of the retinal surface and small wrinkles or folds. More developed PVR is characteristic with fixed folds and retinal detachment. Diagnosis is typically done by indirect ophthalmoscopy and slit-lamp biomicroscopy. Additionally, an ultrasound can help visualize immobile retinal folds of detachment and prominent vitreous membranes. Also, wide-field fundus photography can be used to visualize retinal detachments. However, the clinical history and exam is often enough to make the diagnosis of a retinal detachment.

Development Stages

Ocular wound healing typically occurs in 3 stages: (1) an inflammatory stage, (2) a proliferative stage, and (3) a modulatory stage. PVR can be viewed in a similar fashion, with the wound being the retinal detachment. This healing response often takes place over many weeks. Early on, preretinal PVR adopts an immature appearance and consistency. During this phase, the retina may still remain compliant, and the PVR membrane may be difficult to remove due to its amorphous form. By 6 to 8 weeks, however, the PVR membrane becomes more mature, taking on a white, fibrotic appearance. In this stage, the PVR is more easily identifiable, causes rigidity of the retina, and can be more identifiably removed.

Classification

The extent of PVR in patients is often classified (or graded) depending on the severity. The most commonly used classification system was published by the Retina Society Terminology Committee. It classifies the appearance of PVR based on clinical signs and its geographic location (Grade A, B, C, or D). Grade A is characterized by the appearance of vitreous haze and RPE cells in the vitreous, or by pigment clumping. Grade B is characterized by wrinkling of the edges of the retinal tear or the inner retinal surface. Grade C is characterized by posterior or anterior full thickness retinal folds with the presence of epi/subretinal membranes/bands. Grade D is characterized by fixed retinal folds in all four quadrants. Diagnosis via clinical examination and imaging for PVR is known in the art, e.g., as described in the classification of retinal detachment with proliferative vitreoretinopathy. Ophthalmology, 1983; 90(2): p. 121-5. Clinical examination and classification schemes are further described in Di Lauro et al., J Ophthalmol. 2016; Volume 2016, Article ID 7807596, 6 pages 2016: 7807596. (PMCID: PMC4939352); hereby incorporated by reference.

PVR is Distinct from Proliferative Diabetic Retinopathy

PVR is a condition distinct from proliferative diabetic retinopathy (PDR). PVR is a condition distinct from a small blood vessel disease. The fundamental process involved in

PDR is aberrant angiogenesis, and therefore impacting vascular endothelial cells. To the contrary, in PVR, the fundamental processes is the aberrant epithelial to mesenchymal transition (EMT) of retinal pigment epithelial derived cells, and other cells within the eye. Accordingly, the disclosure herein provides the surprising discovery that targeting RUNX1 may be used as a therapeutic target for the management of PVR.

### Retinal Pigment Eepithelium

The retinal pigment epithelium (RPE) is the pigmented cell layer just outside the neurosensory retina that nourishes retinal visual cells, and is firmly attached to the underlying choroid and overlying retinal visual cells. The RPE forms a monolayer of cells beneath the sensory retina that is normally mitotically inactive except when it is participating in retinal wound repair, where it plays a central role. When wound repair is complete, the RPE usually stops proliferating; failure to do so can result in blinding disorders such as proliferative vitreoretinopathy (PVR) and disciform scarring. For instance, after detachment of the sensory retina, the RPE changes in morphology and begins to proliferate. Multilayered colonies of dedifferentiated RPE cells are formed. Cells then begin to migrate into the subretinal space where they engulf rod outer segments. In some instances, cells migrate onto the surface of the retina and form epiretinal membranes. These events have been implicated in the pathogenesis of proliferative vitreoretinopathy, severe scarring occurring in association with macular degeneration, and poor or delayed recovery of vision after retinal reattachment. Despite these important consequences, little is known about the stimuli involved in RPE dedifferentiation and loss of density-dependent growth control.

Other conditions associated with EMT including cancer, e.g., mesothelioma, Ocular Chronic Graft-Versus-Host Disease, corneal scarring, corneal epithelial downgrowth, conjunctival scarring, eye tumors like melanoma, ocular fibrosis, fibrosis, and complication of glaucoma surgery such as fibrosis (post surgical fibrosis as described in, e.g., Current and Future Techniques in Wound Healing Modulation after Glaucoma Filtering Surgeries. Masoumpour M B, et al. Open Ophthalmol J. 2016. Open Ophthalmol J. 2016 Feb 29;10:68-85. doi: 10.2174/1874364101610010068. eCollection 2016) as well as fibrosis and glaucoma (Friedlander et al., J Clin Invest. 2007, Mar. 1; 117(3): 576-586. Published online 2007 Mar. 1. doi: [10.1172/JC131030] PMCID: MC1804382 PMID). RUNX1 inhibition is useful for reduction, treatment, or prevention of aberrant or pathological EMT occurring in the eye. For example, the methods described herein are used for reducing proliferation and migration of cells within the eye undergoing epithelial to mesenchymal transition, e.g., inhibitors are administered to subjects diagnosed with, suffering from, or having EMT-associated diseases of pathologic ocular fibrosis and proliferation. Diseases include but are not limited to: conjunctival fibrosis (e.g. ocular cicatricial pemphigoid), corneal scarring, corneal epithelial down growth, and/or aberrant post-surgical fibrosis (e.g. after glaucoma surgery, cataract surgery, LASIK, or any intraocular surgery).

### Runt-Related Transcription Factor 1

Runt-related transcription factor 1 (RUNX1), also known as acute myeloid leukemia 1 protein (AML1) or core-binding factor subunit alpha-2 (CBFA2), is a protein that in humans is encoded by the RUNX1 gene.

RUNX1 is a transcription factor that regulates the differentiation of hematopoietic stem cells into mature blood cells. RUNX1 also plays a role in the development of the neurons that transmit pain. It belongs to the Runt-related transcription factor (RUNX) family of genes which are also called core binding factor-α (CBFα). RUNX proteins form a heterodimeric complex with core binding factor β (CBFβ) which confers increased deoxyribonucleic acid (DNA) binding and stability to the complex.

In humans, the RUNX1 gene is 260 kilobases (kb) in length, and is located on chromosome 21 (21q22.12). The gene can be transcribed from 2 alternative promoters, promoter 1 (distal) or promoter 2 (proximal). As a result, various isoforms of RUNX1 can be synthesized, facilitated by alternative splicing. The full-length RUNX1 protein is encoded by 12 exons. Among the exons are two defined domains, namely the runt homology domain (RHD) or the runt domain (exons 2, 3 and 4), and the transactivation domain (TAD) (exon 6). These domains are necessary for RUNX1 to mediate DNA binding and protein-protein interactions respectively. The transcription of RUNX1 is regulated by 2 enhancers (regulatory element 1 and regulatory element 2), and these tissue specific enhancers enable the binding of lymphoid or erythroid regulatory proteins, therefore the gene activity of RUNX1 is highly active in the hematopoietic system.

An exemplary isoform of RUNX1 (Q01196-1; SEQ ID NO: 1) has 453 amino acids. As a transcription factor (TF), its DNA binding ability is encoded by the runt domain (residues 50-177 of SEQ ID NO: 1), which is homologous to the p53 family. Without wishing to be bound by any scientific theory, the runt domain of RUNX1 is believed to bind to the core consensus sequence TGTGGNNN of SEQ ID NO: 1 (where NNN can represent either TTT or TCA). DNA recognition is achieved by loops of the 12-stranded β-barrel and the C-terminus “tail” (residues 170-177 of SEQ ID NO: 1), which clamp around the sugar phosphate backbone and fits into the major and minor grooves of DNA. Specificity is achieved by making direct or water-mediated contacts with the bases. RUNX1 can bind DNA as a monomer, but its DNA binding affinity is enhanced by 10 fold if it heterodimerizes with the CBFβ, also via the runt domain. The RUNX family is often referred to as α-subunits, together with binding of a common β-subunit CBFβ, RUNX can behave as heterodimeric transcription factors collectively called the core binding factors (CBFs).

An amino acid sequence for human RUNX1 is publically available in the UniProt database under accession number Q01196-1 (SEQ ID NO: 1) and is as follows:

MRIPVDASTSRRFTPPSTALSPGKMSEALPLGAPDAGAALAGKLRSGDRS
MVEVLADHPGELVRTDSPNFLCSVLPTHWRCNKTLPIAFKVVALGDVPDG
TLVTVMAGNDENYSAELRNATAAMKNQVARFNDLRFVGRSGRGKSFTLTI
TVFTNPPQVATYHRAIKITVDGPREPRRHRQKLDDQTKPGSLSFSERLSE
LEQLRRTAMRVSPHHPAPTPNPRASLNHSTAFNPQPQSQMQDTRQIQPSP
PWSYDQSYQYLGSIASPSVHPATPISPGRASGMTTLSAELSSRLSTAPDL
TAFSDPRQFPALPSISDPRMHYPGAFTYSPTPVTSGIGIGMSAMGSATRY
HTYLPPPYPGSSQAQGGPFQASSPSYHLYYGASAGSYQFSMVGGERSPPR
ILPPCTNASTGSALLNPSLPNQSDVVEAEGSHSNSPTNMAPSARLEEAVW
RPY

Amino acid sequences of additional isoforms are publically available in the UniProt database under accession numbers Q01196-2 (SEQ ID NO: 2); Q01196-3 (SEQ ID NO: 3); Q01196-4 (SEQ ID NO: 4); Q01196-5 (SEQ ID NO: 5); Q01196-6 (SEQ ID NO: 6); Q01196-7 (SEQ ID NO: 7); Q01196-8 (SEQ ID NO: 8); Q01196-9 (SEQ ID NO: 9); Q01196-10 (SEQ ID NO: 10); and Q01196-11 (SEQ ID NO: 11).

Exemplary landmark sequences and domains include, residues 80-84 (DNA binding domain), residues 135-143 (DNA binding domain), residues 168-177 (DNA binding domain), residues 291-371 (interaction with K(lysine) acetyltransferase 6A (KATAGA)), residues 307-400 (interaction with K(lysine) acetyltransferase 6B (KATA6B)), and residues 362-402 (interaction with forkhead box P3 (FOXP3)).

A nucleotide sequence that encodes human RUNX1 is publically available in the GenBank database under accession number NM_001001890.2 (SEQ ID NO: 12) and is as follows (start and stop codon are bolded and underlined):

CATAGAGCCAGCGGGCGCGGGCGGGACGGGCGCCCCGCGGCCGGACCCAG
CCAGGGCACCACGCTGCCCGGCCCTGCGCCGCCAGGCACTTCTTTCCGGG
GCTCCTAGGGACGCCAGAAGGAAGTCAACCTCTGCTGCTTCTCCTTGGCC
TGCGTTGGACCTTCCTTTTTTTGTTGTTTTTTTTTGTTTTTCCCCTTTCT
TCCTTTTGAATTAACTGGCTTCTTGGCTGGATGTTTTCAACTTCTTTCCT
GGCTGCGAACTTTTCCCCAATTGTTTTCCTTTTACAACAGGGGGAGAAAG
TGCTCTGTGGTCCGAGGCGAGCCGTGAAGTTGCGTGTGCGTGGCAGTGTG
CGTGGCAGGATGTGCGTGCGTGTGTAACCCGAGCCGCCCGATCTGTTTCG
ATCTGCGCCGCGGAGCCCTCCCTCAAGGCCCGCTCCACCTGCTGCGGTTA
CGCGGCGCTCGTGGGTGTTCGTGCCTCGGAGCAGCTAACCGGCGGGTGCT
GGGCGACGGTGGAGGAGTATCGTCTCGCTGCTGCCCGAGTCAGGGCTGAG
TCACCCAGCTGATGTAGACAGTGGCTGCCTTCCGAAGAGTGCGTGTTTGC
ATGTGTGTGACTCTGCGGCTGCTCAACTCCCAACAAACCAGAGGACCAGC
CACAAACTTAACCAACATCCCCAAACCCGAGTTCACAGATGTGGGAGAGC
TGTAGAACCCTGAGTGTCATCGACTGGGCCTTCTTATGATTGTTGTTTTA
AGATTAGCTGAAGATCTCTGAAACGCTGAATTTTCTGCACTGAGCGTTTT
GACAGAATTCATTGAGAGAACAGAGAACATGACAAGTACTTCTAGCTCAG
CACTGCTCCAACTACTGAAGCTGATTTTCAAGGCTACTTAAAAAAATCTG
CAGCGTACATTAATGGATTTCTGTTGTGTTTAAATTCTCCACAGATTGTA
TTGTAAATATTTTATGAAGTAGAGCATATGTATATATTTATATATACGTG
CACATACATTAGTAGCACTACCTTTGGAAGTCTCAGCTCTTGCTTTTCGG
GACTGAAGCCAGTTTTGCATGATAAAAGTGGCCTTGTTACGGGAGATAAT
TGTGTTCTGTTGGGACTTTAGACAAAACTCACCTGCAAAAAACTGACAGG
CATTAACTACTGGAACTTCCAAATAATGTGTTTGCTGATCGTTTTACTCT
TCGCATAAATATTTTAGGAAGTGTATGAGAATTTTGCCTTCAGGAACTTT
TCTAACAGCCAAAGACAGAACTTAACCTCTGCAAGCAAGATTCGTGGAAG
ATAGTCTCCACTTTTTAATGCACTAAGCAATCGGTTGCTAGGAGCCCATC
CTGGGTCAGAGGCCGATCCGCAGAACCAGAACGTTTTCCCCTCCTGGACT
GTTAGTAACTTAGTCTCCCTCCTCCCCTAACCACCCCCGCCCCCCCCCAC
CCCCCGCAGTAATAAAGGCCCCTGAACGTGTATGTTGGTCTCCCGGGAGC
TGCTTGCTGAAGATCCGCGCCCCTGTCGCCGTCTGGTAGGAGCTGTTTGC
AGGGTCCTAACTCAATCGGCTTGTTGTGATGCGTATCCCCGTAGATGCCA
GCACGAGCCGCCGCTTCACGCCGCCTTCCACCGCGCTGAGCCCAGGCAAG
ATGAGCGAGGCGTTGCCGCTGGGCGCCCCGGACGCCGGCGCTGCCCTGGC
CGGCAAGCTGAGGAGCGGCGACCGCAGCATGGTGGAGGTGCTGGCCGACC
ACCCGGGCGAGCTGGTGCGCACCGACAGCCCCAACTTCCTCTGCTCCGTG
CTGCCTACGCACTGGCGCTGCAACAAGACCCTGCCCATCGCTTTCAAGGT
GGTGGCCCTAGGGGATGTTCCAGATGGCACTCTGGTCACTGTGATGGCTG
GCAATGATGAAAACTACTCGGCTGAGCTGAGAAATGCTACCGCAGCCATG
AAGAACCAGGTTGCAAGATTTAATGACCTCAGGTTTGTCGGTCGAAGTGG
AAGAGGGAAAAGCTTCACTCTGACCATCACTGTCTTCACAAACCCACCGC
AAGTCGCCACCTACCACAGAGCCATCAAAATCACAGTGGATGGGCCCCGA
GAACCTCGAAGACATCGGCAGAAACTAGATGATCAGACCAAGCCCGGGAG
CTTGTCCTTTTCCGAGCGGCTCAGTGAACTGGAGCAGCTGCGGCGCACAG
CCATGAGGGTCAGCCCACACCACCCAGCCCCCACGCCCAACCCTCGTGCC
TCCCTGAACCACTCCACTGCCTTTAACCCTCAGCCTCAGAGTCAGATGCA
GGATACAAGGCAGATCCAACCATCCCCACCGTGGTCCTACGATCAGTCCT
ACCAATACCTGGGATCCATTGCCTCTCCTTCTGTGCACCCAGCAACGCCC
ATTTCACCTGGACGTGCCAGCGGCATGACAACCCTCTCTGCAGAACTTTC
CAGTCGACTCTCAACGGCACCCGACCTGACAGCGTTCAGCGACCCGCGCC
AGTTCCCCGCGCTGCCCTCCATCTCCGACCCCCGCATGCACTATCCAGGC
GCCTTCACCTACTCCCCGACGCCGGTCACCTCGGGCATCGGCATCGGCAT
GTCGGCCATGGGCTCGGCCACGCGCTACCACACCTACCTGCCGCCGCCCT
ACCCCGGCTCGTCGCAAGCGCAGGGAGGCCCGTTCCAAGCCAGCTCGCCC
TCCTACCACCTGTACTACGGCGCCTCGGCCGGCTCCTACCAGTTCTCCAT
GGTGGGCGGCGAGCGCTCGCCGCCGCGCATCCTGCCGCCCTGCACCAACG
CCTCCACCGGCTCCGCGCTGCTCAACCCCAGCCTCCCGAACCAGAGCGAC
GTGGTGGAGGCCGAGGGCAGCCACAGCAACTCCCCCACCAACATGGCGCC
CTCCGCGCGCCTGGAGGAGGCCGTGTGGAGGCCCTACTGAGGCGCCAGGC
CTGGCCCGGCTGGGCCCCGCGGGCCGCCGCCTTCGCCTCCGGGCGCGCGG
GCCTCCTGTTCGCGACAAGCCCGCCGGGATCCCGGGCCCTGGGCCCGGCC
ACCGTCCTGGGGCCGAGGGCGCCCGACGGCCAGGATCTCGCTGTAGGTCA
GGCCCGCGCAGCCTCCTGCGCCCAGAAGCCCACGCCGCCGCCGTCTGCTG
GCGCCCCGGCCCTCGCGGAGGTGTCCGAGGCGACGCACCTCGAGGGTGTC
CGCCGGCCCCAGCACCCAGGGGACGCGCTGGAAAGCAAACAGGAAGATTC
CCGGAGGGAAACTGTGAATGCTTCTGATTTAGCAATGCTGTGAATAAAAA
GAAAGATTTTATACCCTTGACTTAACTTTTTAACCAAGTTGTTTATTCCA
AAGAGTGTGGAATTTTGGTTGGGGTGGGGGGAGAGGAGGGATGCAACTCG
CCCTGTTTGGCATCTAATTCTTATTTTTAATTTTTCCGCACCTTATCAAT
TGCAAAATGCGTATTTGCATTTGGGTGGTTTTTATTTTTATATACGTTTA
TATAAATATATATAAATTGAGCTTGCTTCTTTCTTGCTTTGACCATGGAA
AGAAATATGATTCCCTTTTCTTTAAGTTTTATTTAACTTTTCTTTTGGAC
TTTTGGGTAGTTGTTTTTTTTTGTTTTGTTTTGTTTTTTTGAGAAACAGC
TACAGCTTTGGGTCATTTTTAACTACTGTATTCCCACAAGGAATCCCCAG
ATATTTATGTATCTTGATGTTCAGACATTTATGTGTTGATAATTTTTTAA
TTATTTAAATGTACTTATATTAAGAAAAATATCAAGTACTACATTTTCTT
TTGTTCTTGATAGTAGCCAAAGTTAAATGTATCACATTGAAGAAGGCTAG
AAAAAAAGAATGAGTAATGTGATCGCTTGGTTATCCAGAAGTATTGTTTA
CATTAAACTCCCTTTCATGTTAATCAAACAAGTGAGTAGCTCACGCAGCA
ACGTTTTTAATAGGATTTTTAGACACTGAGGGTCACTCCAAGGATCAGAA
GTATGGAATTTTCTGCCAGGCTCAACAAGGGTCTCATATCTAACTTCCTC
CTTAAAACAGAGAAGGTCAATCTAGTTCCAGAGGGTTGAGGCAGGTGCCA
ATAATTACATCTTTGGAGAGGATTTGATTTCTGCCCAGGGATTTGCTCAC
CCCAAGGTCATCTGATAATTTCACAGATGCTGTGTAACAGAACACAGCCA
AAGTAAACTGTGTAGGGGAGCCACATTTACATAGGAACCAAATCAATGAA
TTTAGGGGTTACGATTATAGCAATTTAAGGGCCCACCAGAAGCAGGCCTC
GAGGAGTCAATTTGCCTCTGTGTGCCTCAGTGGAGACAAGTGGGAAAACA
TGGTCCCACCTGTGCGAGACCCCCTGTCCTGTGCTGCTCACTCAACAACA
TCTTTGTGTTGCTTTCACCAGGCTGAGACCCTACCCTATGGGGTATATGG
GCTTTTACCTGTGCACCAGTGTGACAGGAAAGATTCATGTCACTACTGTC
CGTGGCTACAATTCAAAGGTATCCAATGTCGCTGTAAATTTTATGGCACT
ATTTTTATTGGAGGATTTGGTCAGAATGCAGTTGTTGTACAACTCATAAA
TACTAACTGCTGATTTTGACACATGTGTGCTCCAAATGATCTGGTGGTTA
TTTAACGTACCTCTTAAAATTCGTTGAAACGATTTCAGGTCAACTCTGAA
GAGTATTTGAAAGCAGGACTTCAGAACAGTGTTTGATTTTTATTTTATAA
ATTTAAGCATTCAAATTAGGCAAATCTTTGGCTGCAGGCAGCAAAAACAG
CTGGACTTATTTAAAACAACTTGTTTTTGAGTTTTCTTATATATATATTG
ATTATTTGTTTTACACACATGCAGTAGCACTTTGGTAAGAGTTAAAGAGT
AAAGCAGCTTATGTTGTCAGGTCGTTCTTATCTAGAGAAGAGCTATAGCA
GATCTCGGACAAACTCAGAATATATTCACTTTCATTTTTGACAGGATTCC
CTCCACAACTCAGTTTCATATATTATTCCGTATTACATTTTTGCAGCTAA
ATTACCATAAAATGTCAGCAAATGTAAAAATTTAATTTCTGAAAAGCACC
ATTAGCCCATTTCCCCCAAATTAAACGTAAATGTTTTTTTTCAGCACATG
TTACCATGTCTGACCTGCAAAAATGCTGGAGAAAAATGAAGGAAAAAATT
ATGTTTTTCAGTTTAATTCTGTTAACTGAAGATATTCCAACTCAAAACCA
GCCTCATGCTCTGATTAGATAATCTTTTACATTGAACCTTTACTCTCAAA
GCCATGTGTGGAGGGGGCTTGTCACTATTGTAGGCTCACTGGATTGGTCA
TTTAGAGTTTCACAGACTCTTACCAGCATATATAGTATTTAATTGTTTCA
AAAAAAATCAAACTGTAGTTGTTTTGGCGATAGGTCTCACGCAACACATT
TTTGTATGTGTGTGTGTGTGCGTGTGTGTGTGTGTGTGTGAAAAATTGCA
TTCATTGACTTCAGGTAGATTAAGGTATCTTTTTATTCATTGCCCTCAGG
AAAGTTAAGGTATCAATGAGACCCTTAAGCCAATCATGTAATAACTGCAT
GTGTCTGGTCCAGGAGAAGTATTGAATAAGCCATTTCTACTGCTTACTCA
TGTCCCTATTTATGATTTCAACATGGATACATATTTCAGTTCTTTCTTTT
TCTCACTATCTGAAAATACATTTCCCTCCCTCTCTTCCCCCCAATATCTC
CCTTTTTTTCTCTCTTCCTCTATCTTCCAAACCCCACTTTCTCCCTCCTC
CTTTTCCTGTGTTCTCTTAAGCAGATAGCACATACCCCCACCCAGTACCA
AATTTCAGAACACAAGAAGGTCCAGTTCTTCCCCCTTCACATAAAGGAAC
ATGGTTTGTCAGCCTTTCTCCTGTTTATGGGTTTCTTCCAGCAGAACAGA
GACATTGCCAACCATATTGGATCTGCTTGCTGTCCAAACCAGCAAACTTT
CCTGGGCAAATCACAATCAGTGAGTAAATAGACAGCCTTTCTGCTGCCTT
GGGTTTCTGTGCAGATAAACAGAAATGCTCTGATTAGAAAGGAAATGAAT
GGTTCCACTCAAATGTCCTGCAATTTAGGATTGCAGATTTCTGCCTTGAA
ATACCTGTTTCTTTGGGACATTCCGTCCTGATGATTTTTATTTTTGTTGG
TTTTTATTTTTGGGGGGAATGACATGTTTGGGTCTTTTATACATGAAAAT
TTGTTTGACAATAATCTCACAAAACATATTTTACATCTGAACAAAATGCC
TTTTTGTTTACCGTAGCGTATACATTTGTTTTGGGATTTTTGTGTGTTTG
TTGGGAATTTTGTTTTTAGCCAGGTCAGTATTGATGAGGCTGATCATTTG
GCTCTTTTTTTCCTTCCAGAAGAGTTGCATCAACAAAGTTAATTGTATTT
ATGTATGTAAATAGATTTTAAGCTTCATTATAAAATATTGTTAATGCCTA
TAACTTTTTTTCAATTTTTTTGTGTGTGTTTCTAAGGACTTTTTCTTAGG
TTTGCTAAATACTGTAGGGAAAAAAATGCTTCTTTCTACTTTGTTTATTT
TAGACTTTAAAATGAGCTACTTCTTATTCACTTTTGTAAACAGCTAATAG
CATGGTTCCAATTTTTTTTAAGTTCACTTTTTTTGTTCTAGGGGAAATGA
ATGTGCAAAAAAAGAAAAAGAACTGTTGGTTATTTGTGTTATTCTGGATG
TATAAAAATCAATGGAAAAAAATAAACTTTCAAATTGAAATGACGGTATA
ACACATCTACTGAAAAAGCAACGGGAAATGTGGTCCTATTTAAGCCAGCC
CCCACCTAGGGTCTATTTGTGTGGCAGTTATTGGGTTTGGTCACAAAACA
TCCTGAAAATTCGTGCGTGGGCTTCTTTCTCCCTGGTACAAACGTATGGA
ATGCTTCTTAAAGGGGAACTGTCAAGCTGGTGTCTTCAGCCAGATGACAT
GAGAGAATATCCCAGAACCCTCTCTCCAAGGTGTTTCTAGATAGCACAGG
AGAGCAGGCACTGCACTGTCCACAGTCCACGGTACACAGTCGGGTGGGCC
GCCTCCCCTCTCCTGGGAGCATTCGTCGTGCCCAGCCTGAGCAGGGCAGC
TGGACTGCTGCTGTTCAGGAGCCACCAGAGCCTTCCTCTCTTTGTACCAC
AGTTTCTTCTGTAAATCCAGTGTTACAATCAGTGTGAATGGCAAATAAAC
AGTTTGACAAGTACATACACCATA

Additional RUNX1-encoding nucleotide sequences are publically available in the GenBank database under accession numbers NM_001754.4 (homo sapiens, SEQ ID NO: 13); NM_001122607.1 (homo sapiens, SEQ ID NO: 14); XM_005261068.3 (homo sapiens, SEQ ID NO: 15); XM_011529770.2 (homo sapiens, SEQ ID NO: 16); XR_937576.2 (homo sapiens, SEQ ID NO: 17); XM_011529768.2 (homo sapiens, SEQ ID NO: 18); XM_005261069.4 (homo sapiens, SEQ ID NO: 19); XM_017028487.1 (homo sapiens, SEQ ID NO: 20); XM_011529767.2 (homo sapiens, SEQ ID NO: 21); and XM_011529766.2 (homo sapiens, SEQ ID NO: 22).

### Runt-Related Transcription Factor 2

Runt-related transcription factor 2 (RUNX2), also known as core-binding factor subunit alpha-1, is a protein that in humans is encoded by the RUNX2 gene. RUNX2 has been is a transcription factor that has been associated with osteoblast differentiation.

An amino acid sequence for human RUNX2 is publically available in the GenBank Database under accession number NP_001019801.3 (SEQ ID NO: 23), and is as follows:

| | |
|---|---|
| 1 | masnslfstv tpcqqnffwd pstsrrfspp ssslqpgkms |
| | dvspvvaaqq qqqqqqqqqq |
| 61 | qqqqqqqqqq qeaaaaaaaa aaaaaaaaav prlrpphdnr |
| | tmveiiadhp aelvrtdspn |
| 121 | flcsvlpshw rcnktlpvaf kvvalgevpd gtvvtvmagn |
| | denysaelrn asavmknqva |
| 181 | rfndlrfvgr sgrgksftlt itvftnppqv atyhraikvt |
| | vdgpreprrh rqklddskps |
| 241 | lfsdrlsdlg riphpsmrvg vppqnprpsl nsapspfnpq |
| | gqsqitdprq aqssppwsyd |
| 301 | qsypsylsqm tspsihsttp lsstrgtglp aitdvprris |
| | dddtatsdfc lwpstlskks |
| 361 | qagaselgpf sdprqfpsis sltesrfsnp rmhypatfty |
| | tppvtsgmsl gmsatthyht |
| 421 | ylpppypgss qsqsgpfqts stpylyygts sgsyqfpmvp |
| | ggdrspsrml ppctttsngs |
| 481 | tllnpnlpnq ndgvdadgsh sssptvlnss grmdesvwrp |
| | y |

Exemplary landmark sequences and domains include, residues 49-71 (polyglutamine repeat), residues 73-89 (polyalanine repeat), residues 109-230 (runt domain), residues 242-258 (domain for interaction with forkhead Box O1 (FOXO1)), residues 336-439 (domain for interaction with K(lysine) acetyltransferase 6A (KATA6A)), residues 374-488 (domain for interaction with K(lysine) acetyltransferase 6B (KATA6B)), and residues 430-521 (RUNX1 inhibition domain).

Additional amino acid sequences of human RUNX2 isoforms are publically available in the GenBank database under accession numbers: NP_001015051.3 (homo sapiens, SEQ ID NO: 24), Q13950.2 (homo sapiens, SEQ ID NO: 25), and NP_001265407.1 (homo sapiens SEQ ID NO: 26). Amino acid sequences of additional RUNX2 isoforms are publically available in the GenBank database under accession numbers NP_001139392.1 (mus musculus, SEQ ID NO: 27) NP_001139510.1 (mus musculus, SEQ ID NO: 28), NP 001258556.1 (mus musculus, SEQ ID NO: 29), NP_ 001258559.1 (mus musculus, SEQ ID NO: 30), and NP_001258560.1 (mus musculus, SEQ ID NO: 31).

Nucleic acids of additional RUNX2 human isoforms are publically available in the GenBank database under accession numbers: NM_001015051.3 (homo sapiens, SEQ ID NO: 32), NM 001024630.3 (homo sapiens, SEQ ID NO: 33), and NM_001278478.1 (homo sapiens, SEQ ID NO: 34). Nucleic acid sequences of additional RUNX2 isoforms are publically available in the GenBank database under accession numbers NM_001145920.2 (mus musculus, SEQ ID NO: 35), NM_001146038.2 (mus musculus, SEQ ID NO: 36), NM_001271627.1 (mus musculus, SEQ ID NO: 37), NM_001271630.1 (mus musculus, SEQ ID NO: 38), and NM_001271631.1 (mus musculus, SEQ ID NO: 39).

### Exemplary Inhibitors

Aspects of the present subject matter relate to the administration of an RUNX1 inhibitor. In various embodiments, an inhibitor may be, e.g., an aptamer, an oligonucleotide (e.g., an antisense oligonucleotide, a ribozyme, or an RNA interfering molecule), a peptide, an antibody or a fragment thereof, or a small molecule, that specifically binds to RUNX1 or a polynucleotide that encodes RUNX1.

Small Molecule CBFβ-RUNX1 Inhibitors

In various embodiments, the RUNX1 inhibitor is a small molecule inhibitor. Non-limiting examples include:

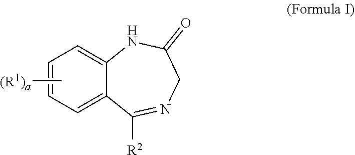

or pharmaceutically acceptable salts or esters thereof, wherein each R¹ is individually selected from halogen, alkyl, aryl, heteroaryl, or alkoxy; R² is selected from aryl or heteroaryl; and a is 0 to 4;

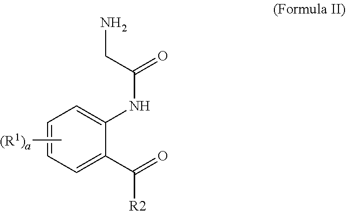

or a pharmaceutically acceptable salt or ester thereof, wherein each R¹ is individually selected from halogen, alkyl, aryl, heteroaryl, or alkoxy; R² is selected from aryl or heteroaryl; and a is 0 to 4; or

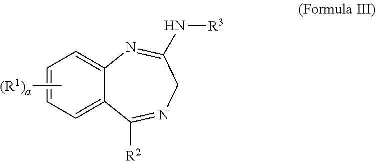

or a pharmaceutically acceptable salt or ester thereof, wherein each R¹ is individually selected from halogen, alkyl, aryl, heteroaryl, or alkoxy; R2 is selected from aryl or heteroaryl; R³ is alkyl or aryl; and a is 0 to 4.

In certain embodiments of formulae I-III, R² is a heteroaryl, particularly pyrrolyl, and especially pyrrol-2-yl. In certain embodiments of formulae I-III, R¹ is a halogen, particularly Cl or F. In certain embodiments of formula III, R³ is a lower alkyl. In certain embodiments of formulae I-III, R² is a heteroaryl, particularly pyrrolyl, and especially pyrrol-2-yl; and R¹ is a halogen, particularly Cl or F. In certain embodiments of formula III, R2 is a heteroaryl, particularly pyrrolyl, and especially pyrrol-2-yl; R¹ is a halogen, particularly Cl or F; and R³ is a lower alkyl.

The term “alkoxy” refers to a group of the formula —OR, wherein R is an organic group such as an alkyl group, optionally substituted with an alkenyl, alkynyl, aryl, aralkyl, cycloalkyl, halogenated alkyl, or heterocycloalkyl group. Suitable alkoxy groups include methoxy, ethoxy, n-propoxy, i-propoxy, n-butoxy, i-butoxy, sec-butoxy, tert-butoxy cyclopropoxy, cyclohexyloxy, and the like.

The term “alkyl” refers to a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 10 carbon atoms. Alkyl groups may be substituted alkyls wherein one or more hydrogen atoms are substituted with a substituent such as halogen, cycloalkyl, alkoxy, amino, hydroxyl, aryl, or carboxyl. For example, an “alkoxyalkyl” has the structure —ROR, wherein R is an alkyl group.

The term “aryl” refers to any carbon-based aromatic group including, but not limited to, benzyl, naphthyl, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorous. The aryl group can be optionally substituted with one or more groups including, but not limited to, alkyl, alkynyl, alkenyl, aryl, halide, nitro, amino, ester, ketone, aldehyde, hydroxy, carboxylic acid, or alkoxy, or the aryl group can be unsubstituted.

The term “heteroaryl” refers to a mono- or poly-cyclic (e.g., bi-, or tri-cyclic or more) fused or non-fused, radical or ring system having at least one aromatic ring, having from five to ten ring atoms of which one ring atom is selected from S, O and N; zero, one or two ring atoms are additional heteroatoms independently selected from S, O and N; and the remaining ring atoms are carbon. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

The term “derivative” refers to a compound or portion of a compound that is derived from or is theoretically derivable from a parent compound.

The term “pharmaceutically acceptable salt or ester” refers to salts or esters prepared by conventional means that include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. “Pharmaceutically acceptable salts” of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of “pharmacologically acceptable salts,” see Berge et al., J. Pharm. Sci. 66:1 (1977). “Pharmaceutically acceptable esters” includes those derived from compounds described herein that are modified to include a hydroxy or a carboxyl group. An in vivo hydrolysable ester is an ester, which is hydrolysed in the human or animal body to produce the parent acid or alcohol. Suitable pharmaceutically acceptable esters for carboxy include C₁₋₆ alkoxymethyl esters for example methoxy-methyl, C₁₋₆ alkanoyloxymethyl esters for example pivaloyloxymethyl, phthalidyl esters, C₃₋₈ cycloalkoxycarbonyloxy C₁₋₆ alkyl esters for example 1-cyclohexylcarbonyl-oxyethyl; 1,3-dioxolen-2-onylmethyl esters for example 5-methyl-1,3-dioxolen-2-onylmethyl; and C₁₋₆ alkoxycarbonyloxyethyl esters for example 1-methoxycarbonyl-oxyethyl which may be formed at any carboxy group in the compounds.

In some embodiments relating to a small molecule inhibitor that binds RUNX1, the small molecule inhibitor comprises Ro5-3335, Ro24-7429, NSC140873, MLS000548294, MLS001048862, or NSC156594. See, e.g., Cunningham et al. (2012) Proc Natl Acad Sci USA, 109(36): 14592-14597,Haubrich, R. et al., J Infect Dis 172(5): 1246-52, and U.S. Patent Application Publication No. 2014/0004082, the entire contents of each of which are incorporated herein by reference. Additional examples of RUNX1 inhibitors are described in U.S. Pat. Nos. 5,641,773; 5,164,376; 5,141,735; 5,041,438; 5,036,101; and 3,405,122, as well as U.S. Patent Application Publication No. 2014/0004082, the entire contents of each of which are hereby incorporated herein by reference.

Ro5-3335 has the following structure:

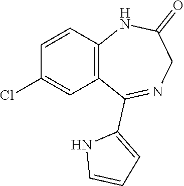 [SMILES: O=C1CN=C(c2ccc[nH]2)c2cc(Cl)ccc2N1]

The CAS Registry Number for Ro5-3335 is 30195-30-3.

Ro24-7429 has the following structure:

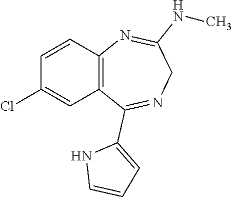 [SMILES: CNC1=Nc2ccc(Cl)cc2C(c2ccc[nH]2)=NC1]

The CAS Registry Number for Ro24-7429 is 13939-45-0.

NSC140873 has the following structure:

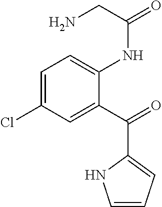 [SMILES: NCC(=O)Nc1ccc(Cl)cc1C(=O)c1ccc[nH]1]

The CAS Registry Number for NSC140873 is 106410-13-3.

MLS000548294 has the following structure:

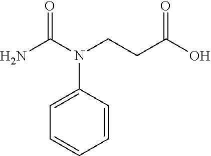 [SMILES: NC(=O)N(CCC(=O)O)c1ccccc1]

The PubChem ID for MLS000548294 is 768985.

MLS001048862 has the following structure:

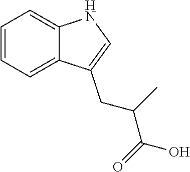

The PubChem ID for MLS001048862 is 2772042.

NSC156594 has the following structure:

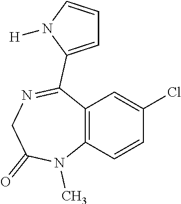 [SMILES: CN1C(=O)CN=C(c2ccc[nH]2)c2cc(Cl)ccc21]

The PubChem ID for NSC156594 is 457993.

The synthesis of several of the compounds disclosed above and analogs thereof have been previously described, for example, in U.S. Pat. Nos. 5,641,773; 5,164,376; 5,141,735: 5,041,438; 5,036,101; and 3,405,122, the entire contents of each of which are incorporated herein by reference.

In some embodiments, the RUNX1 inhibitor inhibits RUNX1 via inhibition of CBFβ, which is the transcriptional partner of RUNX1.

Non-limiting examples of CBFβ inhibitors include:

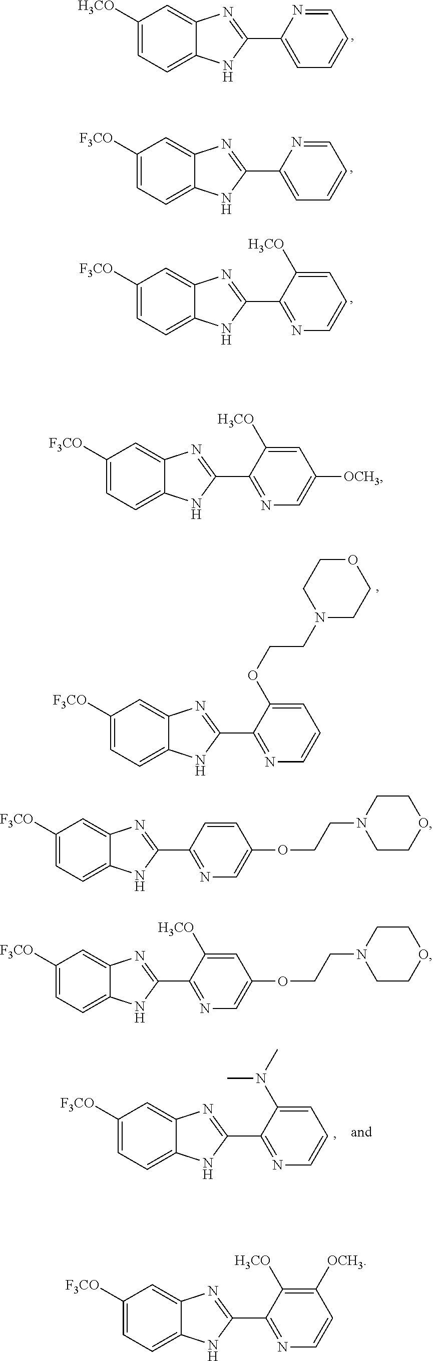

Non-limiting descriptions of CBFβ inhibitors and aspects thereof are described in Illendula et al. (2016) EBioMedicine 8: 117-131, the entire content of which is incorporated herein by reference. In some examples, the inhibitor comprises a pyridyl benzimidazole.

Proteins and Peptides

In some embodiments, a protein, peptide, or a fragment thereof is used to inhibit RUNX1. A non-limiting example of such an inhibitor for RUNX1 is a dominant negative CBF-Beta protein (CBFB-MYH11). See, e.g., Castilla et al. (1996) Cell. 1996;87:687-696, the entire content of which is hereby incorporated herein by reference.

### RNA

In some embodiments, a RNA is used to encode a protein that inhibits RUNX1 or the RNA itself has inhibitory properties.

### Aptamers

Aptamers are small, single stranded biomolecules, typically oligonucleotides (either DNA or RNA) or peptides, that bind to a specific target molecule (e.g. a protein or small molecule such as a steroid). They can be considered analogous to antibodies in their specificity but, unlike antibodies, aptamers are have a relatively low molecular weight. Peptide-based aptamers are generally less than thirty residues long while nucleotide-based aptamers are typically less than one hundred residues long.

Non-limiting examples of methods that are useful for designing aptamers that target a particular protein, such as RUNX1, are described in U.S. Pat. Nos. 8,484,010; 5,582,981: PCT International Patent Application No. WO 2015/049356; Blackwell et al., (1993) Science 250:1104-1110; Blackwell, et al., (1990) Science 250:1149-1152; Tuerk and Gold (1990) Science 249:505-510; and Joyce (1989) Gene 82:83-87, the entire contents of each of which are incorporated herein by reference.

Antisense Oligonucleotides

As used herein, an “antisense oligonucleotide” is an oligonucleotide that inhibits gene expression by a mechanism other than RNAi. Non-limiting examples of antisense oligonucleotides which decrease the amount of RUNX1 produced by cells that can be employed in the methods described herein include antisense oligonucleotides that are complementary (e.g., at least about 90, 95, 96, 97, 98, 99, or 100% complementary) to a stretch of at least about 10, 11, 12, 13, 14, 15, 16, 17, 18, 19, 20, 21, 22, 23, 24, 25. 26, 27, 28, 29 or 30 consecutive nucleotides having a sequence found within a nucleotide sequence that encodes RUNX1, such as any of the RUNX1-encoding nucleotide sequences disclosed herein.

Antisense oligonucleotides comprise nucleotide sequences which are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long. Longer sequences also can be used. Antisense oligonucleotide molecules can be provided in a DNA construct and introduced into a cell as described above to decrease the level of RUNX1 gene products in the cell.

Antisense oligonucleotides can comprise deoxyribonucleotides, ribonucleotides, or a combination of both. Antisense oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters.

Modifications of gene expression can be obtained by designing antisense oligonucleotides which will form duplexes to the control, 5′, or regulatory regions of the gene. Antisense oligonucleotides that target the transcription initiation site, e.g., between positions −10 and +10 from the start site, are used in some embodiments. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (Nicholls et al., 1993, J Immunol Meth 165:81-91). Antisense oligonucleotides that are complementary to a sequence that includes the translational start site, and/or that are complementary to a portion of a target mRNA within 10 nucleotides of the translational start site, are used in various embodiments. An antisense oligonucleotide also can be designed to block translation of mRNA by preventing the transcript from binding to ribosomes.

Precise complementarity is not required for successful complex formation between an antisense oligonucleotide and the complementary sequence of a RUNX1 polynucleotide. Antisense oligonucleotides which comprise, for example, 2, 3, 4, or 5 or more stretches of contiguous nucleotides which are precisely complementary to a RUNX1, each separated by a stretch of contiguous nucleotides which are not complementary to adjacent RUNX1 nucleotides, can provide sufficient targeting specificity for RUNX1 mRNA. In some embodiments, each stretch of complementary contiguous nucleotides is at least 4, 5, 6, 7, or 8 or more nucleotides in length. Noncomplementary intervening sequences may be, e.g., 1, 2, 3, or 4 nucleotides in length. One skilled in the art can easily use the calculated melting point of an antisense-sense pair to determine the degree of mismatching which will be tolerated between a particular antisense oligonucleotide and a particular RUNX1 polynucleotide sequence. Antisense oligonucleotides can be modified without affecting their ability to hybridize to a RUNX1 polynucleotide. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′ or 5′-substituted oligonucleotide in which the 3′ hydroxyl group and/or the 5′ phosphate group is substituted, also can be employed in a modified antisense oligonucleotide. These modified antisense oligonucleotides can be prepared by methods well known in the art.

Ribozymes

Ribozymes are RNA molecules with catalytic activity (Uhlmann et al., 1987, Tetrahedron. Lett. 215, 3539-3542). Ribozymes can be used to inhibit gene function by cleaving an RNA sequence, as is known in the art. The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences. The coding sequence of a polynucleotide can be used to generate ribozymes which will specifically bind to mRNA transcribed from the polynucleotide. Methods of designing and constructing ribozymes which can cleave other RNA molecules in trans in a highly sequence specific manner have been developed and described in the art. For example, the cleavage activity of ribozymes can be targeted to specific RNAs by engineering a discrete “hybridization” region into the ribozyme. The hybridization region contains a sequence complementary to the target RNA and thus specifically hybridizes with the target RNA.

Specific ribozyme cleavage sites within an RNA target can be identified by scanning the target molecule for ribozyme cleavage sites which include the following sequences: QUA, GUU, and GUC. Once identified, short RNA sequences of between 15 and 20 ribonucleotides corresponding to the region of the target RNA containing the cleavage site can be evaluated for secondary structural features which may render the target inoperable. Suitability of candidate RNA targets also can be evaluated by testing accessibility to hybridization with complementary oligonucleotides using ribonuclease protection assays. The nucleotide sequences shown in SEQ ID NOs: 1-26 and their complements provide sources of suitable hybridization region sequences. Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease RUNX1 expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer element, and a transcriptional terminator signal, for controlling transcription of ribozymes in the cells (U.S. Pat. No. 5,641,673). Ribozymes also can be engineered to provide an additional level of regulation, so that destruction of mRNA occurs only when both a ribozyme and a target gene are induced in the cells.

RNA Interference

As used herein, an “RNA interference” inducing compound refers to a compound capable of inducing RNA interference or “RNAi” of target gene (e.g., RUNX1) expression, depending on the context. RNAi involves mRNA degradation. The use of RNAi has been described in Fire et al. (1998) Nature 19;391(6669):806-11. Elbashir et al. (2001) EMBO J. 20(23): 6877-6888, and Cheloufi et al. (2010) Nature 465, 584-589, the entire contents of each of which are incorporated herein by reference.

Isolated RNA molecules can mediate RNAi. That is, the isolated RNA molecules of the present subject matter mediate degradation or block expression of mRNA that is the transcriptional product of the gene, which is also referred to as a target gene. For convenience, such mRNA may also be referred to herein as mRNA to be degraded. RNAi molecules may be, e.g., double-stranded RNA, small interfering RNA (siRNA), hairpin RNA. microRNA molecules which may be altered compared to naturally occurring RNA by the addition, deletion, substitution and/or alteration of one or more nucleotides. Such alterations can include addition of non-nucleotide material, such as to the end(s) of the RNA or internally (at one or more nucleotides of the RNA). Nucleotides in the RNA molecules of the present invention can also comprise nonstandard nucleotides, including non-naturally occurring nucleotides or deoxyribonucleotides. Collectively, all such altered RNAi molecules may be referred to as analogs or analogs of naturally-occurring RNA. RNA of the present invention need only be sufficiently similar to natural RNA that it has the ability to mediate RNAi.

As used herein the phrase “mediate RNAi” refers to and indicates the ability to distinguish which mRNA molecules are to be afflicted with the RNAi machinery or process. RNA that mediates RNAi interacts with the RNAi machinery such that it directs the machinery to degrade particular mRNAs or to otherwise reduce the expression of the target protein. In some embodiments, the present invention relates to RNA molecules that direct cleavage of specific mRNA to which their sequence corresponds. It is not necessary that there be perfect correspondence of the sequences, but the correspondence must be sufficient to enable the RNA to direct RNAi inhibition by cleavage or blocking expression of the target mRNA. In some embodiments, an RNAi molecule comprises a stretch of about 16 to 29, 18 to 23, 21-23, or at least 12, 13, 14, 15, 16, 17, 18, 19, 20, 21, 22, 23, 24, or 25 nucleotides having a sequence that is at least about 90. 95, 96, 97, 98, 99, or 100% complementary to a target sequence. As noted above, the RNA molecules of the present invention may comprise an RNA portion and some additional portion, for example a deoxyribonucleotide portion.

Antibodies

In some embodiments, the RUNX1 inhibitor is an antibody or a fragment thereof.

As used herein, the term “antibody” refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimerie, single chain, F_{ab}, F_{ab′} and F_{(ab′)2} fragments, an Fab expression library, single-chain antibody molecules (e.g., scFv), and multispecific antibodies formed from antibody fragments. By “specifically bind” or “immunoreacts with” is meant that the antibody reacts with one or more antigenic determinants of the desired antigen and does not react (i.e., bind) with other polypeptides or binds at much lower affinity (K_{d}>10⁻⁶) with other polypeptides.

An “antibody fragment” refers to a molecule other than an intact antibody that comprises a portion of an intact antibody that binds the antigen to which the intact antibody binds. The terms “full length antibody.” “intact antibody,” and “whole antibody” are used herein interchangeably to refer to an antibody having a structure substantially similar to a native antibody structure or having heavy chains that contain an Fc region.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ea., 2nd ed. Raven Press, N.Y. (1989)). The variable regions of each light/heavy chain pair form the antibody binding site.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs.” Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three hypervariable regions (HVRs) of each variable domain interact to define an antigen binding site on the surface of the VH-VL dimer. Collectively, the six HVRs or a subset thereof confer antigen binding specificity to the antibody. However, even a single variable domain (or half of an Fv comprising only three HVRs specific for an antigen) has the ability to recognize and bind antigen, although usually at a lower affinity than the entire binding site.

A “Fab” fragment contains a variable and constant domain of the light chain and a variable domain and the first constant domain (CHI) of the heavy chain. F(ab′) 2 antibody fragments comprise a pair of Fab fragments which are generally covalently linked near their carboxy termini by hinge cysteines between them. Other chemical couplings of antibody fragments are also known in the art,

“Single-chain Fv” or “scFv” antibody fragments comprise the VH and VL domains of an antibody, wherein these domains are present in a single polypeptide chain. Generally the Fv polypeptide further comprises a polypeptide linker between the VH and L domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-31S (1994).

The term “diabodies” refers to small antibody fragments with two antigen-binding sites, which fragments comprise a heavy chain variable domain (VH) connected to a light chain variable domain (VL) in the same polypeptide chain (VH and VL). By using a linker that is too short to allow pairing between the two domains on the same chain, the domains are forced to pair with the complementary domains of another chain and create two antigen-binding sites. Diabodies are described more fully in, for example, BP 404,097; WO 93/11161; and Hollinger et al ., Proc . Natl. Acad. Sci. USA, 90:6444-6448 (1993).

The expression “linear antibodies” refers to the antibodies described in Zapata et al., Protein Eng. 8 (10): 1057-1062 (1995). Briefly, these antibodies comprise a pair of tandem segments which, together with complementary light chain polypeptides, form a pair of antigen binding regions. Linear antibodies can be bispecific or monospecific.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used may be made by a variety of techniques, including but not limited to the hybridoma method, recombinant DNA methods, phage-display methods, and methods utilizing transgenic animals containing all or part of the human immunoglobulin loci. such methods and other exemplary methods for making monoclonal antibodies being described herein.

As used herein, the term “epitope” includes any protein determinant capable of specific binding to an antibody, an antibody fragment, or a T-cell receptor. Epitopic determinants usually consist of chemically active surface groupings of molecules such as amino acids or sugar side chains and usually have specific three dimensional structural characteristics, as well as specific charge characteristics. An antibody is said to specifically bind an antigen when the dissociation constant is ≤1 μM; preferably ≤100 nM and most preferably ≤10 nM.

Antibodies can be produced according to any method known in the art.

Methods of preparing monoclonal antibodies are known in the art. For example, monoclonal antibodies may be prepared using hybridoma methods, such as those described by Kohler and Milstein (1975) Nature 256:495. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a full length protein or a fragment thereof. Generally. either peripheral blood lymphocytes (“PBLs”) are used if cells of human origin are desired. or spleen cells or lymph node cells are used if non-human mammalian sources are desired. The lymphocytes are then fused with an immortalized cell line using a suitable fusing agent, such as polyethylene glycol, to form a hybridoma cell (see pp. 59-103 in Goding (1986) Monoclonal Antibodies: Principles and Practice Academic Press). Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HPRT-deficient cells.

In some examples, the antibodies to an epitope for an interested protein as described herein or a fragment thereof are humanized antibodies. Humanized forms of non-human (e.g., murine) antibodies are chimeric molecules of immunoglobulins, immunoglobulin chains or fragments thereof (such as Fv, Fab, Fats, Fiabe or other antigen-binding subsequences of antibodies) which contain minimal sequence derived from non-human immunoglobulin.

Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al. 1986. Nature 321:522-525; Riechmann et al. 1988. Nature 332:323-329; Presta. 1992. Curr. Op. Struct. Biol. 2:593-596). Humanization can be essentially performed following methods of Winter and co-workers (see, e.g., Jones et al. 1986. Nature 321:522-525; Riechmann et al. 1988. Nature 332:323-327; and Verhoeven et al. 1988. Science 239:1534-1536), by substituting rodent CDRs or CDR sequences for the corresponding sequences of a human antibody. Accordingly, such humanized antibodies are chimeric antibodies (e.g., U.S. Pat. No. 4,816,567), wherein substantially less than an intact human variable domain has been substituted by the corresponding sequence from a non-human species.

In various examples the antibodies to an epitope of an interested protein as described herein or a fragment thereof are human antibodies. Human antibodies can also be produced using various techniques known in the art. including phage display libraries (Hoogenboom and Winter. 1991. J. Mol. Biol. 227:381-388; Marks et al. 1991. J. Mol. Biol. 222:581-597) or the preparation of human monoclonal antibodies [e.g., Cole et al. 1985. Monoclonal Antibodies and Cancer Therapy Liss; Boerner et al. 1991. J. Immunol. 147(1):86-95]. Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in most respects, including gene rearrangement. assembly, and antibody repertoire. This approach is described, e.g., in U.S. Pat. Nos. 5,545,807: 5.545,806; 5,569,825; 5,625,126: 5,633,425; 5,661,016, and in the following scientific publications: Marks et al. 1992. Bio/Technology 10:779-783; Lonberg et al. 1994. Nature 368:856-859; Morrison. 1994. Nature 368:812-13; Fishwild et al. 1996. Nature Biotechnology 14:845-51; Neuberger. 1996. Nature Biotechnology 14:826; Lonberg and Huszar. 1995. Intern. Rev. Immunol. 13:65-93. U.S. Pat. No. 6,719,971 also provides guidance to methods of generating humanized antibodies.

In some embodiments, an intrabody is used to inhibit RUNX1. An “intrabody” (from intracellular and antibody) is an antibody that works within the cell to bind to an intracellular antigen. Intrabodies typically lack disulfide bonds and are capable of modulating the expression or activity of target genes through their specific binding activity. Intrabodies include single domain fragments such as isolated VH and VL domains and scFvs. An intrabody can include sub-cellular trafficking signals attached to the N or C terminus of the intrabody to allow expression at high concentrations in the sub-cellular compartments where a target protein is located. Upon interaction with a target gene, an intrabody modulates target protein function and/or achieves phenotypic/functional knockout by mechanisms such as accelerating target protein degradation and sequestering the target protein in a non-physiological sub-cellular compartment. Other mechanisms of intrabody-mediated gene inactivation can depend on the epitope to which the intrabody is directed, such as binding to the catalytic site on a target protein or to epitopes that are involved in protein-protein, protein-DNA, or protein-RNA interactions. In various embodiments, the intrabody is expressed within a target cell, e.g., by a viral or plasmid expression vector that has been introduced into the target cell. An intrabody may remain in the cytoplasm, or it may have a nuclear localization signal, or it may undergo cotranslational translocation across the membrane into the lumen of the endoplasmic reticulum, provided that it is retained in that compartment through a KDEL sequence. Because antibodies ordinarily are designed to be secreted from the cell, intrabodies require special alterations, including the use of single-chain antibodies (scFvs), modification of immunoglobulin VL domains for hyperstability, selection of antibodies resistant to the more reducing intracellular environment, or expression as a fusion protein with maltose binding protein or other stable intracellular proteins. Non-limiting aspects of intrabodies are described, e.g., in U.S. Pat. No. 9.133,269; U.S. Patent Application Publication No. 2006/0034834; Chen et al. (1994) Human gene therapy 5 (5): 595-601; and Shaki-Loewenstein et al. (2005) Journal of immunological methods 303 (1-2): 19-39, the entire contents of each of which are incorporated herein by reference.

Exemplary antibodies against RUNX1 include, but are not limited to, antibodies obtained from Abcam (Cambridge, MA, USA) (e.g., Cat. Nos. ab23980, ab35962, ab 189172, ab 189153, and ab91002), antibodies obtained from Novus Biologicals (Littleton, CO, USA) (e.g., Cat. Nos. NBP1-89105, H00000861-M05, H00000861-M06, MAB2399, and H00000861-M02), and antibodies obtained from ThermoFisher Scientific (Cambridge, MA, USA) (e.g., 710233, MAS-15814, PA 1-41078, OSR00271W, PAS-17434, PAS-19638, PAS-12409, PA5-40076, and PAS-17351).

Gene Therapy

In some embodiments, a gene editing method is used to modulate (e.g., reduce) RUNX1 expression and/or activity. Non-limiting examples of gene editing systems useful in such embodiments include the clustered regularly interspaced short palindromic repeat (CRISPR)-Cas system; zinc finger nuclease (ZFN) systems, and transcription activator-like effector-based nuclease (TALEN) systems.

Exemplary aspects of the CRISPR-Cas system are described in, e.g., U.S. Pat. No. 9,023,649, issued May 5, 2015; U.S. Pat. No. 9,074,199, issued Jul. 7, 2015; and U.S. Pat. No. 8,697,359, issued Apr. 15, 2014 the entire contents of each of which are incorporated herein by reference.

With their highly flexible but specific targeting, CRISPR-Cas systems can be manipulated and redirected to become powerful tools for genome editing. CRISPR-Cas technology permits targeted gene cleavage and gene editing in a variety of eukaryotic cells, and editing can be directed to virtually any genomic locus. Exemplary CRISPR Cas genes include Cas1, Cas2, Cas3′, Cas3″, Cas4, Cas5, Cas6, Cas6e (formerly referred to as CasE, Cse3), Cas6f (i.e., Csy4), Cas7, Cas8a1, Cas8a2, Cas8b, Cas8c, Cas9, Cas10, Cas10d, Csy1, Csy2, CPf1, Csy3, Cse1, Cse2, Cse1, Cse2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1l, Csf2, Csf3, and Csf4. These enzymes are known; for example, the amino acid sequence of Streptococcus pyogenes Cas9 protein may be found in the SwissProt database under accession number Q99ZW2.

Other non-limiting examples of approaches for gene editing include the use of zinc finger nucleases, which are artificial restriction enzymes generated by fusing a zinc finger DNA-binding domain to a DNA-cleavage domain. A zinc finger nuclease is a site-specific endonuclease designed to bind and cleave DNA at specific positions. There are two protein domains. The first domain is the DNA binding domain, which consists of eukaryotic transcription factors and contain the zinc finger. The second domain is the nuclease domain, which consists of the Fokl restriction enzyme and is responsible for the catalytic cleavage of DNA. The DNA-binding domains of individual ZFNs typically contain between three and six individual zinc finger repeats and can each recognize between 9 and 18 basepairs. If the zinc finger domains are perfectly specific for their intended target site then even a pair of 3-finger ZFNs that recognize a total of 18 basepairs can, in theory, target a single locus in a mammalian genome. Various strategies have been developed to engineer Cys2His2 zinc fingers to bind desired sequences. These include both “modular assembly” and selection strategies that employ either phage display or cellular selection systems. The most straightforward method to generate new zinc-finger arrays is to combine smaller zinc-finger “modules” of known specificity. The most common modular assembly process involves combining three separate zinc fingers that can each recognize a 3 basepair DNA sequence to generate a 3-finger array that can recognize a 9 basepair target site. Other procedures can utilize either 1-finger or 2-finger modules to generate zinc-finger arrays with six or more individual zinc fingers. Numerous selection methods have been used to generate zinc-finger arrays capable of targeting desired sequences. Initial selection efforts utilized phage display to select proteins that bound a given DNA target from a large pool of partially randomized zinc-finger arrays. More recent efforts have utilized yeast one-hybrid systems, bacterial one-hybrid and two-hybrid systems, and mammalian cells. The non-specific cleavage domain from the type IIs restriction endonuclease FoKI is typically used as the cleavage domain in ZENs. This cleavage domain must dimerize in order to cleave DNA and thus a pair of ZENs are required to target non-palindromic DNA sites. Standard ZENs fuse the cleavage domain to the C-terminus of each zinc finger domain. In order to allow the two cleavage domains to dimerize and cleave DNA, the two individual ZENs must bind opposite strands of DNA with their C-termini a certain distance apart. The most commonly used linker sequences between the zinc finger domain and the cleavage domain requires the 5′ edge of each binding site to be separated by 5 to 7 bp.

TALENs are restriction enzymes that can be engineered to cut specific sequences of DNA. They are made by fusing a TAL effector DNA-binding domain to a DNA cleavage domain. Transcription activator-like effectors (TALEs) can be engineered to bind practically any desired DNA sequence. so when combined with a nuclease, DNA can be cut at specific locations. The restriction enzymes can be introduced into cells, for use in gene editing or for genome editing in situ. Alongside zinc finger nucleases and CRISPR/Cas9, TALEN is a prominent tool in the field of genome editing.

NOTCH Signaling Inhibition

Notch signaling is an upstream regulator of the expression of Runx genes (Burns CE, et al., Genes and Development, 2005, 19:2331-2342). Modalities for preventing or reducing proliferation or migration of RPE cells is based on inhibition of the Notch receptors (Notch 1, Notch 2, Notch 3, Notch 4) and its ligands (Delta like 1, Delta like 2, Delta like 5, Jagged 1 and Jagged 2) or other modulators of the Notch pathway operate via regulation of RUNX1. These modalities of treatment include inhibition of gamma-secretase, modulating antibodies against the receptors and the ligands, and other small molecules, biologicals and genetic approaches that inhibit RUNX1 expression via modulation of Notch signaling activity.

In some embodiments, NOTCH signaling is modulated to reduce RUNX1 function. For example, a NOTCH inhibitor is used to reduce RUNX1 expression or activity. Non-limiting examples of NOTCH inhibitors include aptamers, oligonucleotides (e.g., antisense oligonucleotides, ribozymes, and RNAi molecules), peptides (e.g., a portion of or the entire extracellular domain of a NOTCH protein), antibodies, antibody fragments, and small molecules that specifically bind to a NOTCH protein or a polynucleotide that encodes a NOTCH protein. Non-limiting examples of small molecule inhibitors for NOTCH proteins include compounds having the following structures:

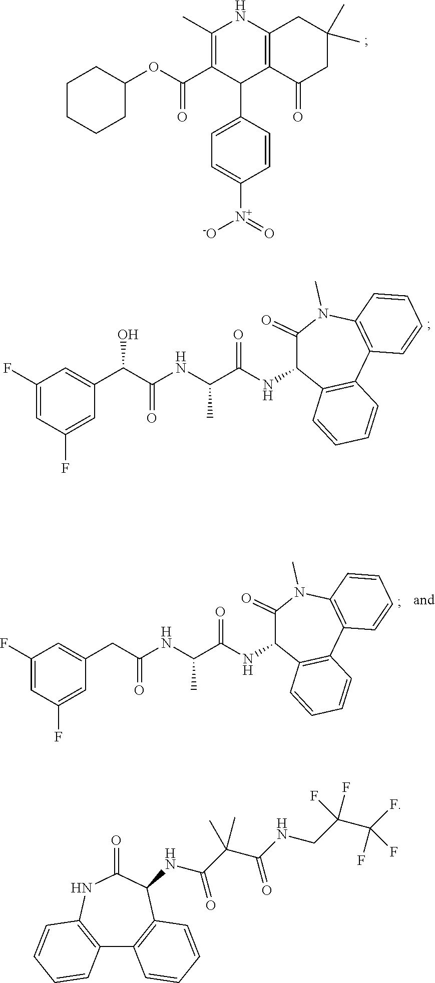

Additional non-limiting examples of NOTCH inhibitors (as well as aspects of NOTCH signaling) are described in Espinoza and Miele, Pharmacol Ther. 2013,139(2): 95-110; and Yuan et al., Cancer Letters 2015, 369(1) 20-27, the entire contents of each of which are incorporated herein in their entireties.

### Immunomodulatory Imide Drug (IMID) Inhibitors

In some examples, the RUNX1 inhibitor comprises an immunomodulatory imide drug (IMiD). IMiDs are a class of immunomodulatory drugs containing an imide group. In non-limiting examples, the IMID includes thalidomide and its analogs, e.g., lenalidomide, pomalidomide, and apremilast.

In various embodiments, the IMiD comprises:

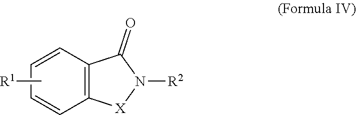

or pharmaceutically acceptable salts or esters thereof, wherein:

R¹═H, NH₂, NHC(O)CH₃

X═CH₂, C(O)

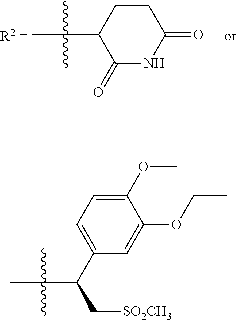 [SMILES: *C1CCC(=O)NC1=O ; *[C@H](CS(C)(=O)=O)c1ccc(OC)c(OCC)c1]

For example, Formula IV encompasses lenalidomide, pomalidomide, thalidomide, and/or apremilast.

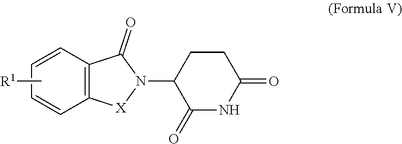

or pharmaceutically acceptable salts or esters thereof, wherein:

R¹═H, NH₂, NHC(O)CH₃

X═CH₂, C(O)

For example, Formula V encompasses lenalidomide, pomalidomide, and/or anthalidomide.

In some examples, the IMID is lenalidomide. The IUPAC name for lenalidomide is 3-(7-amino-3-oxo-1H-isoindol-2-yl)piperidine-2,6-dione. The CAS Registry Number for lenalidomide is 191732-72-6.

Lenalidomide has the following structure:

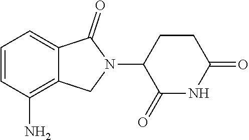 [SMILES: Nc1cccc2c1CN(C1CCC(=O)NC1=O)C2=O]

In some examples, the IMiD is thalidomide. The IUPAC name for thalidomide is 2-(2,6-dioxopiperidin-3-yl)isoindole-1,3-dione. The CAS Registry Number for thalidomide is 50-35-1.

Thalidomide has the following structure:

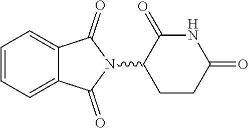 [SMILES: O=C1CCC(N2C(=O)c3ccccc3C2=O)C(=O)N1]

In some examples, the IMID is pomalidomide. The IUPAC name for pomalidomide is 4-amino-2-(2,6-dioxopiperidin-3-yl)isoindole-1,3-dione. The CAS Registry Number for pomalidomide is 19171-19-8.

Pomalidomide has the following structure:

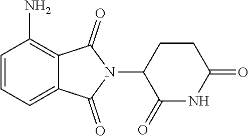 [SMILES: Nc1cccc2c1C(=O)N(C1CCC(=O)NC1=O)C2=O]

In some examples, the IMID is apremilast. The IUPAC name for apremilast is N-[2-[(1S)-1-(3-ethoxy-4-methoxyphenyl)-2-methylsulfonylethyl]-1,3-dioxoisoindol-4-yl]acetamide. The CAS Registry Number for apremilast is 608141-41-9.

Apremilast has the following structure:

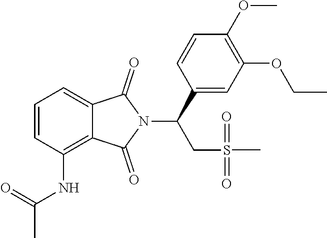 [SMILES: CCOc1cc([C@@H](CS(C)(=O)=O)N2C(=O)c3cccc(NC(C)=O)c3C2=O)ccc1OC]

In other examples, the IMiD comprises phosphodiesterase type 4 (PDE4) inhibitor, e.g., rolipram The IUPAC name for rolipram is 4-(3-cyclopentyloxy-4-methoxyphenyl)pyrrolidin-2-one. The CAS Registry Number for rolipram is 61413-54-5.

Rolipram has the following structure:

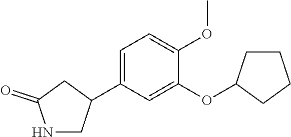 [SMILES: COc1ccc(C2CNC(=O)C2)cc1OC1CCCC1]

The RUNX1 inhibitors may have one or more chiral centers, and thus can exist as one or more stereoisomers. Such stereoisomers can exist as a single enantiomer, a mixture of enantiomers, a mixture of diastereomers, or a racemic mixture.

As used herein, the term “stereoisomers” refers to compounds made up of the same atoms having the same bond order but having different three-dimensional arrangements of atoms that are not interchangeable. The three-dimensional structures are called configurations. As used herein, the term “enantiomers” refers to two stereoisomers that are non-superimposable mirror images of one another. As used herein, the term “optical isomer” is equivalent to the term “enantiomer”. As used herein the term “diastereomer” refers to two stereoisomers which are not mirror images but also not superimposable. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981).

Pharmaceutical Formulations and Delivery

Dosages, formulations, dosage volumes, regimens, and methods for administering a RUNX1 inhibitor may vary. Thus, minimum and maximum effective dosages vary depending on the method of administration.

“Administering” an inhibitor described herein can be effected or performed using any of the various methods and delivery systems known to those skilled in the art. The administering can be, for example, intravenous, oral, ocular (e.g., subconjunctival, intravitreal, retrobulbar, or intracameral), intramuscular, intravascular, intra-arterial, intracoronary, intramyocardial, intraperitoneal, subcutaneous, inhaled, or intrathecal. Other non-limiting examples include topical administration, or coating of a device to be placed within the subject. Topical administration also includes administration of the inhibitor(s) by eye drop, e.g., contacting the surface of the eye with a liquid (aqueous, lipid, or combination thereof) or gel formulation. In other embodiments, administration is carried by injection. e.g., using a needle, or via a catheter.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

As used herein, a “monotherapy” is therapy that is administered to inhibit, treat, or prevent a disorder, such as proliferation or migration of retinal pigment epithelial (RPE) in a subject who comprise a retinal hole or a retinal tear, such as found in PVR), without any other therapy that is used to treat the disorder. A monotherapy for treating a disorder may optionally be combined with another treatment that is used to ameliorate a symptom of the disorder while not being directed against the disorder, for example an analgesic compound. an antipyretic compound, and/or an anti-inflammatory compound (e.g., aspirin, ibuprofen, naproxen, or acetaminophen) may be administered concurrently with the monotherapy. In various embodiments of the invention, a composition comprising a RUNX1 inhibitor may be administered only once or multiple times. For example, a RUNX1 inhibitor may be administered using a method disclosed herein at least about once, twice, three times, four times, five times, six times, or seven times per day, week, month, or year. In some embodiments, a composition comprising a RUNX1 inhibitor is administered once per month. In certain embodiments, the composition is administered once per week, once every two weeks, once a month via intravitreal injection. In various embodiments, such as embodiments involving eye drops, a composition is self-administered.

For the treatment of an ocular disorder, a RUNX1 inhibitor (e.g., a pharmaceutical composition comprising a RUNX1 inhibitor) may be administered locally, e.g., as a topical eye drop, peri-ocular injection (e.g., sub-tenon), intraocular injection, intravitreal injection, retrobulbar injection, intraretinal injection, subretinal injection, suprachoroidal. subconjunctival injection, or using iontophoresis, or peri-ocular devices which can actively or passively deliver drug.

Sustained release of drug may be achieved by the use of technologies such as implants (e.g., solid implants) (which may or may not be bio-degradable) or bio-degradable polymeric matrices (e.g., micro-particles). These may be administered, e.g., peri-ocularly or intravitreally.

Pharmaceutical formulations adapted for topical administration may be formulated as aqueous solutions, ointments, creams, suspensions, lotions, powders, solutions, pastes, gels, sprays, aerosols, liposomes, microcapsules, microspheres, or oils. For treatments of the eye or other external tissues, such as the mouth or skin, the formulations (e.g., a pharmaceutical composition comprising a RUNX1 inhibitor) may be applied as a topical ointment or cream. When formulated in an ointment, a RUNX1 inhibitor may be employed with either a paraffinic or a water-miscible ointment base. Alternatively, a RUNX1 inhibitor may be formulated in a cream with an oil-in-water cream base or a water-in-oil base.

The present subject matter provides compositions comprising a RUNX1 inhibitor and a carrier or excipient suitable for administration to ocular tissue. Such carriers and excipients are suitable for administration to ocular tissue (e.g., sclera, lens, iris, cornea, uvea, retina, macula, or vitreous tissue) without undue adverse side effects (such as toxicity, irritation, and allergic response) commensurate with a reasonable benefit/risk ratio. 20) Pharmaceutical formulations adapted for topical administrations to the eye include eye drops wherein a RUNX1 inhibitor is dissolved or suspended in a suitable carrier, especially an aqueous solvent. Formulations to be administered to the eye will have ophthalmically compatible pH and osmolality. The term “ophthalmically acceptable vehicle” means a pharmaceutical composition having physical properties (e.g., pH and/or osmolality) that are physiologically compatible with ophthalmic tissues.

In some embodiments, an ophthalmic composition of the present invention is formulated as sterile aqueous solutions having an osmolality of from about 200 to about 400 milliosmoles/kilogram water (“mOsm/kg”) and a physiologically compatible pH. The osmolality of the solutions may be adjusted by means of conventional agents, such as inorganic salts (e.g., NaCl), organic salts (e.g., sodium citrate), polyhydric alcohols (e.g., propylene glycol or sorbitol) or combinations thereof.

In various embodiments, the ophthalmic formulations of the present invention may be in the form of liquid, solid or semisolid dosage form. The ophthalmic formulations of the present invention may comprise, depending on the final dosage form, suitable ophthalmically acceptable excipients. In some embodiments, the ophthalmic formulations are formulated to maintain a physiologically tolerable pH range. In certain embodiments, the pH range of the ophthalmic formulation is in the range of from about 5 to about 9. In some embodiments, pH range of the ophthalmic formulation is in the range of from about 6 to about 8, or is about 6.5, about 7, or about 7.5.

In some embodiments, the composition is in the form of an aqueous solution, such as one that can be presented in the form of eye drops. By means of a suitable dispenser, a desired dosage of the active agent can be metered by administration of a known number of drops into the eye, such as by one, two, three, four, or five drops.

One or more ophthalmically acceptable pH adjusting agents and/or buffering agents can be included in a composition of the invention, including acids such as acetic, boric, citric, lactic, phosphoric, and hydrochloric acids: bases such as sodium hydroxide, sodium phosphate, sodium borate, sodium citrate, sodium acetate, and sodium lactate; and buffers such as citrate/dextrose, sodium bicarbonate, and ammonium chloride. Such acids, bases, and buffers can be included in an amount required to maintain pH of the composition in an ophthalmically acceptable range. One or more ophthalmically acceptable salts can be included in the composition in an amount sufficient to bring osmolality of the composition into an ophthalmically acceptable range. Such salts include those having sodium, potassium, or ammonium cations and chloride, citrate, ascorbate, borate, phosphate, bicarbonate, sulfate, thiosulfate, or bisulfite anions.

The ocular delivery device may be designed for the controlled release of one or more therapeutic agents with multiple defined release rates and sustained dose kinetics and permeability. Controlled release may be obtained through the design of polymeric matrices incorporating different choices and properties of biodegradable/bioerodable polymers (e.g., poly(ethylene vinyl) acetate (EVA), superhydrolyzed PVA), hydroxyalkyl cellulose (HPC). methylcellulose (MC), hydroxypropyl methyl cellulose (HPMC), polycaprolactone, poly(glycolic) acid, poly(lactic) acid, polyanhydride, of polymer molecular weights, polymer crystallinity, copolymer ratios, processing conditions, surface finish, geometry, excipient addition, and polymeric coatings that will enhance drug diffusion, erosion, dissolution, and osmosis.

Formulations for drug delivery using ocular devices may combine one or more active agents and adjuvants appropriate for the indicated route of administration. For example, a RUNX1 inhibitor (optionally with another agent) may be admixed with any pharmaceutically acceptable excipient, lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, tableted or encapsulated for conventional administration. Alternatively, the compounds may be dissolved in polyethylene glycol, propylene glycol, carboxymethyl cellulose colloidal solutions, ethanol, corn oil, peanut oil, cottonseed oil, sesame oil, tragacanth gum, and/or various buffers. The compounds may also be mixed with compositions of both biodegradable and non-biodegradable polymers, and a carrier or diluent that has a time delay property. Representative examples of biodegradable compositions can include albumin, gelatin, starch, cellulose, dextrans, polysaccharides, poly (D,L-lactide), poly (D),L-lactide-co-glycolide), poly (glycolide), poly (hydroxybutyrate), poly (alkylcarbonate) and poly (orthoesters), and mixtures thereof. Representative examples of non-biodegradable polymers can include EVA copolymers, silicone rubber and poly (methylacrylate), and mixtures thereof.

Pharmaceutical compositions for ocular delivery also include in situ gellable aqueous composition. Such a composition comprises a gelling agent in a concentration effective to promote gelling upon contact with the eye or with lacrimal fluid. Suitable gelling agents include but are not limited to thermosetting polymers. The term “in situ gellable” as used herein includes not only liquids of low viscosity that form gels upon contact with the eye or with lacrimal fluid, but also includes more viscous liquids such as semi-fluid and thixotropic gels that exhibit substantially increased viscosity or gel stiffness upon administration to the eye. See, for example, Ludwig, Adv. Drug Deliv. Rev. 3: 57:1595-639 (2005), the entire content of which is incorporated herein by reference.

Biocompatible implants for placement in the eye have been disclosed in a number of patents, such as U.S. Pat. Nos. 4,521,210; 4,853,224; 4,997,652; 5,164, 188; 5,443,505; 5,501,856: 5.766,242; 5,824,072; 5,869,079: 6,074.661; 6,331,313: 6.369,116; 6,699,493; and 8,293,210, the entire contents of each of which are incorporated herein by reference.

The implants may be monolithic, i.e. having the active agent (e.g., a RUNX1 inhibitor) or agents homogenously distributed through the polymeric matrix, or encapsulated, where a reservoir of active agent is encapsulated by the polymeric matrix. Due to ease of manufacture, monolithic implants are usually preferred over encapsulated forms. However, the greater control afforded by the encapsulated, reservoir-type implant may be of benefit in some circumstances, where the therapeutic level of the drug falls within a narrow window. In addition, the therapeutic component, including a RUNX1 inhibitor, may be distributed in a non-homogenous pattern in the matrix. For example, the implant may include a portion that has a greater concentration of a RUNX1 inhibitor relative to a second portion of the implant.

The intraocular implants disclosed herein may have a size of between about 5 um and about 2 mm, or between about 10 um and about 1 mm for administration with a needle, greater than 1 mm, or greater than 2 mm, such as 3 mm or up to 10 mm, for administration by surgical implantation. The vitreous chamber in humans is able to accommodate relatively large implants of varying geometries, having lengths of, for example, 1 to 10 mm. The implant may be a cylindrical pellet (e.g., rod) with dimensions of about 2 mm×0.75 mm diameter. The implant may be a cylindrical pellet with a length of about 7 mm to about 10 mm, and a diameter of about 0.75 mm to about 1.5 mm.

The implants may also be at least somewhat flexible so as to facilitate both insertion of the implant in the eye, such as in the vitreous, and accommodation of the implant. The total weight of the implant is usually about 250-5000 μg, more preferably about 500-1000 g. For example, an implant may be about 500 ug. or about 1000 ug. For non-human subject, the dimensions and total weight of the implant(s) may be larger or smaller, depending on the type of subject. For example, humans have a vitreous volume of approximately 3.8 ml, compared with approximately 30 ml for horses, and approximately 60-100 ml for elephants. An implant sized for use in a human may be scaled up or down accordingly for other animals, for example, about 8 times larger for an implant for a horse, or about, for example, 26 times larger for an implant for an elephant.

Implants can be prepared where the center may be of one material and the surface may have one or more layers of the same or a different composition, where the layers may be cross-linked, or of a different molecular weight, different density or porosity, or the like. For example, where it is desirable to quickly release an initial bolus of drug, the center may be a polylactate coated with a polylactate-polyglycolate copolymer, so as to enhance the rate of initial degradation. Alternatively, the center may be polyvinyl alcohol coated with polylactate, so that upon degradation of the polylactate exterior the center would dissolve and be rapidly washed out of the eye.

The implants may be of any geometry including fibers, sheets, films, microspheres, spheres, circular discs, plaques, and the like. The upper limit for the implant size will be determined by factors such as toleration for the implant, size limitations on insertion, ease of handling, etc. Where sheets or films are employed, the sheets or films will be in the range of at least about 0.5 mm×0.5 mm, usually about 3-10 mm×5-10 mm with a thickness of about 0.1-1.0 mm for ease of handling. Where fibers are employed, the fiber diameter will generally be in the range of about 0.05 to 3 nim and the fiber length will generally be in the range of about 0.5-10 mm. Spheres may be in the range of 0.5 um to 4 mm in diameter, with comparable volumes for other shaped particles.

The size and form of the implant can also be used to control the rate of release, period of treatment, and drug concentration at the site of implantation. Larger implants will deliver a proportionately larger dose, but depending on the surface to mass ratio, may have a slower release rate. The particular size and geometry of the implant are chosen to suit the site of implantation.

Microspheres for ocular delivery are described, for example, in U.S. Pat. Nos. 5,837,226; 5,731,005; 5,641,750; 7,354,574; and U.S. Pub. No. 2008-0131484, the entire contents of each of which are incorporated herein by reference.

For oral or enteral formulations for use with the present invention, tablets can be formulated in accordance with conventional procedures employing solid carriers well-known in the art. Capsules employed for oral formulations to be used with the methods of the present invention can be made from any pharmaceutically acceptable material, such as gelatin or cellulose derivatives. Sustained release oral delivery systems and/or enteric coatings for orally administered dosage forms are also contemplated, such as those described in U.S. Pat. Nos. 4,704,295; 4, 556,552; 4,309,404; and 4,309,406, the entire contents of each of which are incorporated herein by reference.

### General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C:” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible

It is understood that where a parameter range is provided, all integers within that range, and tenths thereof, are also provided by the invention. For example, “0.2-5 mg” is a disclosure of 0.2 mg, 0.3 mg, 0.4 mg. 0.5 mg, 0.6 mg etc. up to and including 5.0 mg.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds, e.g., nucleic acid molecules, polynucleotides, polypeptides, proteins, or small molecules are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (RNA or DNA) is free of the genes or sequences that flank it in its naturally- occurring state. Similarly, a purified peptide or protein (e.g., identified by a specific amino acid sequence) is free of the amino acids that flank it in its naturally-occurring state. Purified also defines a degree of sterility that is safe for administration to a human subject, e.g ., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

The transitional term “comprising.” which is synonymous with “including.” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “subject,” “patient,” “individual,” and the like as used herein are not intended to be limiting and can be generally interchanged. An individual described as a “subject,” “patient,” “individual,” and the like does not necessarily have a given disease, but may be merely seeking medical advice. The terms “subject,” “patient,” “individual,” and the like as used herein include all members of the animal kingdom that may suffer from the indicated disorder. In some aspects, the subject is a mammal, and in some aspects, the subject is a buman.

As used herein, the singular forms “a,” “an.” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, “treating” encompasses, e.g., inhibition, regression, or stasis of the progression of a disorder. Treating also encompasses the prevention or amelioration of any symptom or symptoms of the disorder. As used herein, “inhibition” of disease progression of a disease complication in a subject means preventing or reducing the disease progression and/or disease complication in the subject.

As used herein, a “symptom” associated with a disorder includes any clinical or laboratory manifestation associated with the disorder, and is not limited to what the subject can feel or observe.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g ., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. Such sequences that are at least about 80% identical are said to be “substantially identical.” In some embodiments, two sequences are 100% identical. In certain embodiments, two sequences are 100% identical over the entire length of one of the sequences (e.g., the shorter of the two sequences where the sequences have different lengths). In various embodiments, identity may refer to the complement of a test sequence. In some embodiments, the identity exists over a region that is at least about 10 to about 100, about 20 to about 75, about 30 to about 50 amino acids or nucleotides in length. In certain embodiments, the identity exists over a region that is at least about 50 amino acids in length, or more preferably over a region that is 100 to 500, 100 to 200, 150 to 200, 175 to 200, 175 to 225, 175 to 250, 200 to 225, 200 to 250 or more amino acids in length.

For sequence comparison, typically one sequence acts as a reference sequence, to which test sequences are compared. In various embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A the “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In various embodiments, a comparison window is the entire length of one or both of two aligned sequences. In some embodiments, two sequences being compared comprise different lengths, and the comparison window is the entire length of the longer or the shorter of the two sequences. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement).

In various embodiments, an algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the

National Center for Biotechnology Information, as known in the art. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W. T. and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989) alignments (B) of 50, expectation (E) of 10, M=5, N=−4, and a comparison of both strands.

A “control” sample or value refers to a sample that serves as a reference, usually a known reference, for comparison to a test sample. For example, a test sample can be taken from a test subject, e.g., a subject in need of diagnosis for a disease, and compared to samples from known conditions, e.g., a subject (or subjects) that does not have the disease (a negative or normal control), or a subject (or subjects) who does have the disease (positive control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

The term, “normal amount” with respect to a compound (e.g., a protein) refers to a normal amount of the protein in an individual known not to be diagnosed with a disease that comprises increased proliferation or migration of RPE cells in a subject who comprises a retinal hole or retinal tear. The amount of a protein can be measured in a test sample and compared to the “normal control” level, utilizing techniques such as reference limits, discrimination limits, or risk defining thresholds to define cutoff points and abnormal values (e.g., for a particular retinal detachment complication or a symptom thereof). The normal control level means the level of one or more proteins or combined protein indices typically found in a subject known not suffering from a disease that comprises increased proliferation or migration of RPE cells in a subject who comprises a retinal hole or retinal tear. Such normal control levels and cutoff points may vary based on whether a protein is used alone or in a formula combining with other proteins into an index. Alternatively, the normal control level can be a database of protein patterns from previously tested subjects who did not develop increased proliferation or migration of RPE cells (in a subject also comprising a retinal hole or retinal tear) or a particular symptom thereof (e.g., in the event the disease develops or a subject already having the disease is tested) over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease (or a symptom thereof) in question or is not at risk for the disease.

Relative to a control level, the level that is determined may an increased level. As used herein, the term “increased” with respect to level (e.g., protein level) refers to any % increase above a control level. In various embodiments, the increased level may be at least or about a 5% increase, at least or about a 10% increase, at least or about a 15% increase, at least or about a 20% increase, at least or about a 2.5% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level

Relative to a control level, the level that is determined may a decreased level. As used herein, the term “decreased” with respect to level (e.g., protein level) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

“Risk” in the context of the present disclosure, relates to the probability that an event will occur over a specific time period, as in the development of a neovascularization disorder or a symptom thereof, and can mean a subject's “absolute” risk or “relative” risk. In various embodiments, a “high risk” subject is a subject who is likely to develop a disease that comprises increased proliferation or migration of RPE cells in a subject who comprises a retinal hole or retinal tear or a symptom thereof within, e.g., about 1, 2, 3, 4, or 5 years. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used [odds are according to the formula p/(1-p) where p is the probability of event and (1-p) is the probability of no event] to no-conversion.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### EMT

Epithelial-mesenchymal transition (EMT) is characterized by a loss of cell adhesion, which leads to constriction and extrusion of new mesenchymal cells. EMT is a process by which epithelial cells lose their cell polarity and cell-cell adhesion, and gain migratory and invasive properties to become mesenchymal stem cells (which are multipotent stromal cells that can differentiate into a variety of cell types. EMT is essential for numerous developmental processes including mesoderm formation and neural tube formation. EMT has also been shown to occur in wound healing, in organ fibrosis and in the initiation of metastasis in cancer progression. EMT, and its reverse process, MET (mesenchymal-epithelial transition) are critical for development of many tissues and organs in the developing embryo, and numerous embryonic events such as gastrulation, neural crest formation, heart valve formation, palatogenesis and myogenesis. Epithelial cells are closely connected to each other by tight junctions, gap junctions and adherens junctions, have an apico-basal polarity, polarization of the actin cytoskeleton and are bound by a basal lamina at their basal surface. Mesenchymal cells, on the other hand, lack this polarization, have a spindle-shaped morphology and interact with each other only through focal points. Epithelial cells express high levels of E-cadherin, whereas mesenchymal cells express those of N-cadherin, fibronectin and vimentin. Thus, EMT entails profound morphological and phenotypic changes to a cell. Based on the biological context, EMT has been categorized into 3 types: developmental (Type I), fibrosis and wound healing (Type II), and cancer (Type III).

Loss of E-cadherin is a fundamental event in EMT. Many transcription factors (TFs) that can repress E-cadherin directly or indirectly are considered as EMT-TF (EMT inducing TFs). SNAI1/Snail 1, SNAI2/Snail 2 (also known as Slug or Zinc finger protein), Zinc finger E-box binding homeobox 1 and 2 (ZEB1 and ZEB2), transcription factor 3 (TCF3) and krueppel-like factor 8 (KLF8) can bind to the E-cadherin promoter and repress its transcription, whereas factors such as Twist (also referred to as class A basic helix-loop-helix protein 38: bHLHa38), Goosecoid, transcription factor 4 (TCF4), homeobox protein Sineoculis homeobox homolog 1 (SIX1) and fork-head box protein C2 (FOXC2) repress E-cadherin indirectly.

Several signaling pathways (transforming growth factor beta (TGF-β), fibroblast growth factor (FGF), epidermal growth factor (EGF), hepatocyte growth factor (HGF), Wnt/beta-catenin and Notch) and hypoxia may induce EMT. In particular, Ras-MAPK (mitogen-activated protein kinases) activates Snail and Slug. Slug triggers the steps of desmosomal disruption, cell spreading, and partial separation at cell-cell borders, which comprise the first and necessary phase of the EMT process.

Wnt signaling pathway regulates EMT in gastrulation, cardiac valve formation and cancer. Activation of Wnt pathway in breast cancer cells induces the EMT regulator SNAIL and upregulates the mesenchymal marker, vimentin. Also, active Wnt/beta-catenin pathway correlates with poor prognosis in breast cancer patients in the clinic. Similarly, TGF-β activates the expression of SNAIL and ZEB to regulate EMT in heart development, palatogenesis, and cancer. The breast cancer bone metastasis has activated TGF-β signaling, which contributes to the formation of these lesions. However, on the other hand, tumor protein 53 (p53), a well-known tumor suppressor, represses EMT by activating the expression of various microRNAs—miR-200 and miR-34 that inhibit the production of protein ZEB and SNAIL, and thus maintain the epithelial phenotype.

After the initial stage of embryogenesis, the implantation of the embryo and the initiation of placenta formation are associated with EMT. The trophoectoderm cells undergo EMT to facilitate the invasion of endometrium and appropriate placenta placement, thus enabling nutrient and gas exchange to the embryo. Later in embryogenesis, during gastrulation. EMT allows the cells to ingress in a specific area of the embryo—the primitive streak in amniotes, and the ventral furrow in Drosophila. The cells in this tissue express E-cadherin and apical-basal polarity.

During wound healing, keratinocytes at the border of the wound undergo EMT and undergo re-epithelialization or MET when the wound is closed. Snail2 expression at the migratory front influences this state, as its overexpression accelerates wound healing. Similarly, in each menstrual cycle, the ovarian surface epithelium undergoes EMT during post-ovulatory wound healing.

Initiation of metastasis requires invasion, which is enabled by EMT. Carcinoma cells in a primary tumor lose cell-cell adhesion mediated by E-cadherin repression and break through the basement membrane with increased invasive properties, and enter the bloodstream through intravasation. Later, when these circulating tumor cells (CTCs) exit the bloodstream to form micro-metastases, they undergo MET for clonal outgrowth at these metastatic sites. Thus, EMT and MET form the initiation and completion of the invasion-metastasis c ascade. At this new metastatic site, the tumor may undergo other processes to optimize growth. For example, EMT has been associated with programmed death ligand 1 (PD-L1) expression, particularly in lung cancer. Increased levels of PD-L1 suppresses the immune system which allows the cancer to spread more easily.

EMT has been shown to be induced by androgen deprivation therapy in metastatic prostate cancer. Activation of EMT programs via inhibition of the androgen axis provides a mechanism by which tumor cells can adapt to promote disease recurrence and progression. Brachyury, Axl (tyrosine protein kinase receptor UFO), MEK, and Aurora kinase A are molecular drivers of these programs, and inhibitors are currently in clinical trials to determine therapeutic applications. Oncogenic protein kinase C iota type (PKC-jota) can promote melanoma cell invasion by activating Vimentin during EMT. PKC-iota inhibition or knockdown resulted an increase E-cadherin and ras homolog gene family. member A (RhoA) levels while decreasing total Vimentin, phophorylated Vimentin (S39) and partitioning defective 6 homolog alpha (Par6) in metastatic melanoma cells. Cells that undergo EMT gain stem cell-like properties, thus giving rise to Cancer Stem Cells (CSCs).

In addition to the treatment of PVR, the compositions and methods described herein can be used for the treatment and prevention of Epithelial-mesenchymal transition (EMT)-associated diseases. For example, EMT-associated diseases comprise pathological ocular fibrosis and pathological ocular proliferation. Additional diseases and conditions are described by Masoumpour, M. et al., in a journal article entitled, “Current and Future Techniques in Wound Healing Modulation after Glaucoma Filtering Surgeries” The Open Ophthalmology Journal, 2016 (10); 68-85, incorporated herein by reference in its entirety. Other exemplary EMT-associated diseases are described in an article by Friedlander. M., entitled, “Fibrosis and diseases of the eye” The Journal of Clinical Investigation 117(3): 576-586 (2007), incorporated herein by reference in its entirety. For example, the article describes diseases in the anterior segment of the eye (e.g., corneal opacification and glaucoma), dystrophies, herpetic keratitis, inflammation (e.g., pterygiun), macula edema, retinal and vitreous hemorrhage, fibrovascular scarring, neovascular glaucoma, age-related macular degeneration (ARMD), diabetic retinopathy (DR), retinopathy of prematurity (ROP), subretinal fibrosis, epiretinal fibrosis, and gliosis.

### Example 1: Expression of RUNX1 in Cells Derived from Human PVR Membranes (C-PVR)

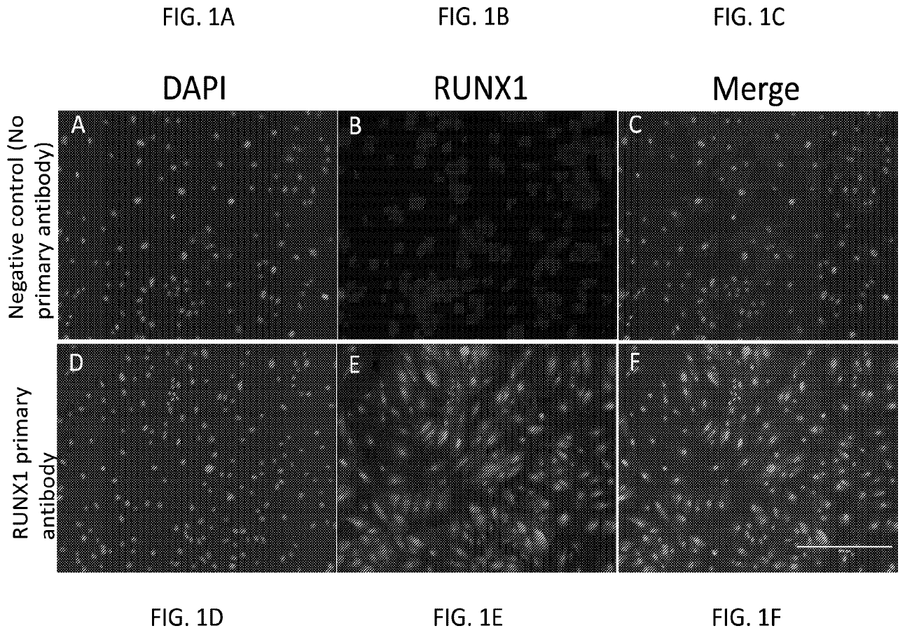

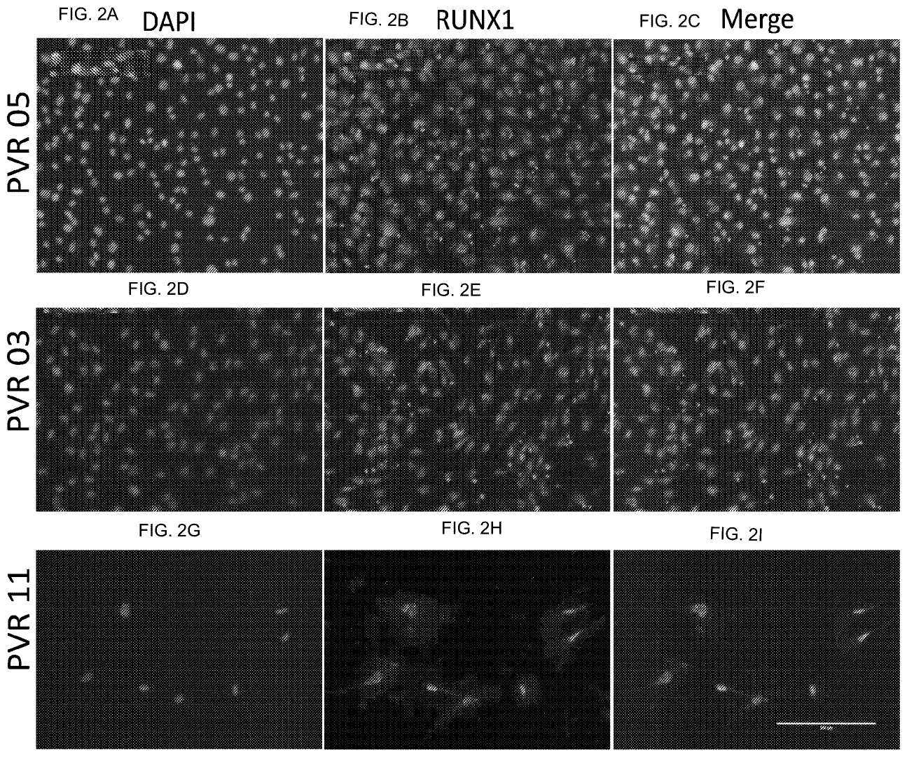

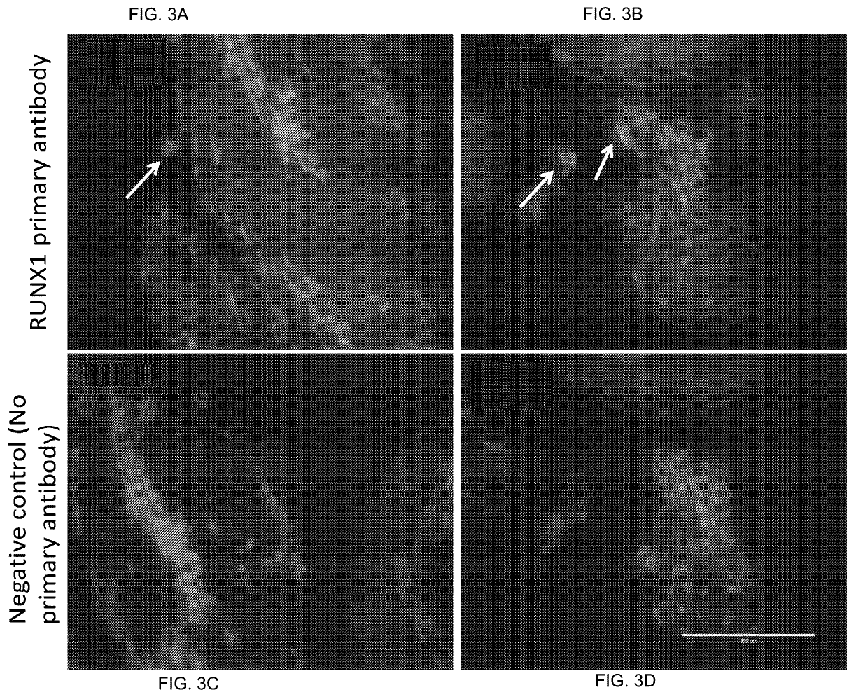

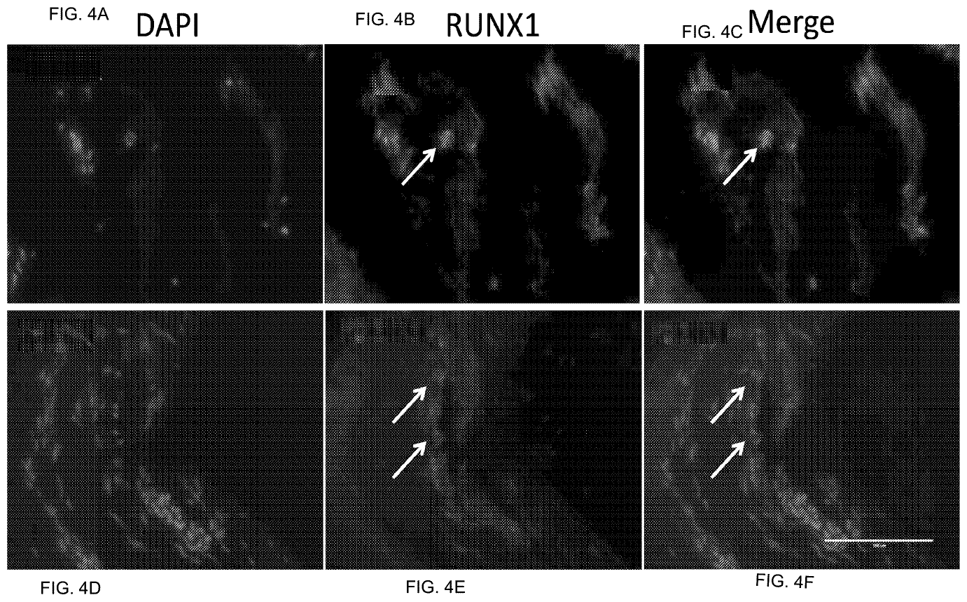

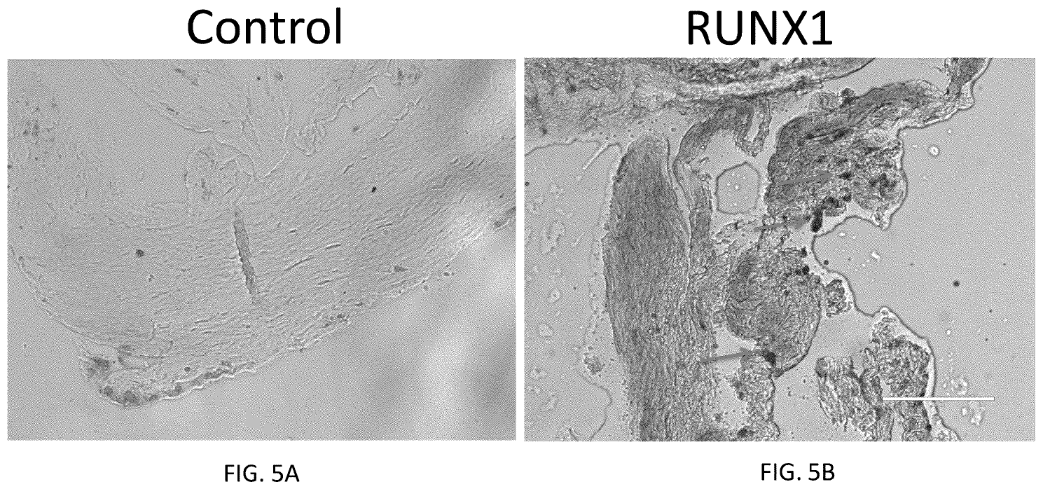

Immunofluorescence staining of RUNX1 in C-PVR cells showed expression of RUNX1 in C-PVR cells (FIG. 1). Immunofluorescence staining of RUNX1 in human PVR membranes (from patients with PVR) showed expression of RUNX1 (FIG. 3A-3D). Additionally, human PVR membrane from a patient (e.g., case 5) showed expression of RUNX1 (FIG. 3A-3D and FIG. 4A-4F). Additionally, immunohistochemistry of human PVR membranes showed RUNX1 expression (FIG. 5A and 5B).

Methods

C-PVR cells were grown in a 48 well plate for 24-72 hours, fixed in 4% paraformaldehyde overnight at 4° C. and stained with rabbit anti-RUNX1 primary antibody (1:100; LifeSpan Biosciences). Cells were incubated with goat anti-rabbit 594 secondary antibody (1:300; Life Technologies) and DAPI for 2 hours, then washed and imaged. Human PVR membrane tissue (e.g., case 5) was freshly obtained and was fixed in 10% formalin overnight and sectioned (6 μm). Serial sections were deparaffinized in 100% xylene, rehydrated in a series of ethanol steps and washed in PBS. Heat-induced epitope retrieval was performed in boiling citrate buffer (pH 6). The slides were blocked and incubated with primary antibody rabbit anti-RUNX1 (1:100; LifeSpan Biosciences) overnight at 4° C. followed by incubation with secondary goat anti-rabbit Alexa Fluor 594 (1:300 Life Technologies) and DAPI for two hours at room temperature. Slides were then washed and imaged (FIG. 3A-3D and 4A-4F).

Immunohistochemistry

Human PVR membrane (e.g., case 5) freshly obtained was fixed in 10% formalin overnight and sectioned (6 μm). Serial sections were deparaffinized in 100% xylene, rebydrated in a series of ethanol steps and washed in PBS. Heat-induced epitope retrieval was performed in boiling citrate buffer (pH 6). The slides were blocked and incubated with primary antibody mouse anti-RUNX1 (1:100); Santa Cruz Biotechnologies) overnight at 4° C. The following day, sections were incubated in a biotinylated secondary antibody (1:300; Life Technologies) followed by tyramide amplification system and 3,3′ Diaminobenzidine chromagen staining. Slides were then washed and imaged (FIG. 5A and 5B).

### Example 2: Inhibition of RUNX1 in Cells Derived from Human PVR Membrane

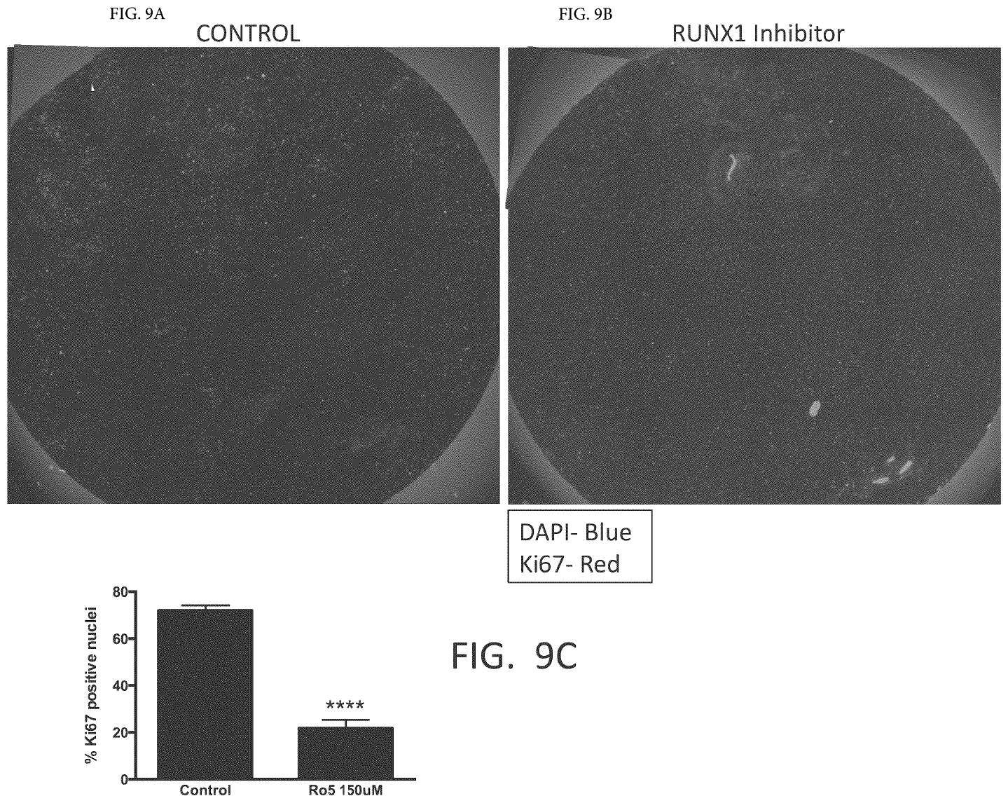

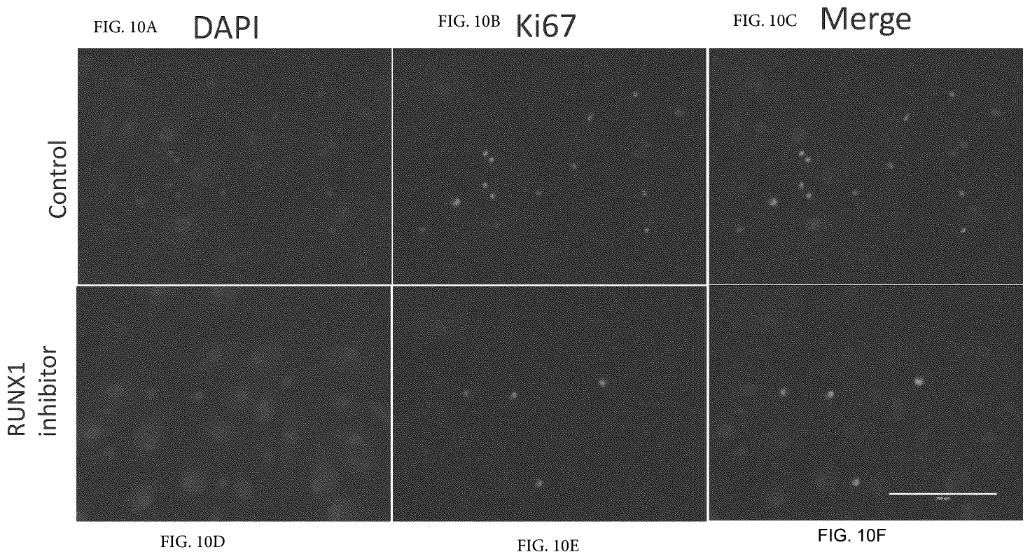

Proliferation of cells derived from human PVR membrane (C-PVR) was inhibited by the RUNX1 inhibitor, Ro5-335 (FIG. 9A-9C). A significant reduction in the cell number and proliferative capacity of C-PVR cells was observed (FIG. 9A-9C). The RUNX1 inhibitor, Ro5-335 reduced the growth of human PVR membranes in an explant model (FIG. 18A-18D). Growth was observed from control explants (FIG. 18A and 18C) as compared to the explants treated with RUNX1 inhibitor, Ro5-335 (FIG. 18B and 18D), which showed now growth after four days.

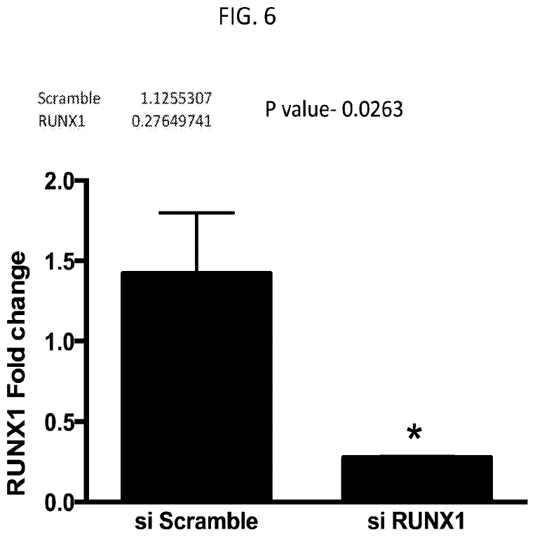

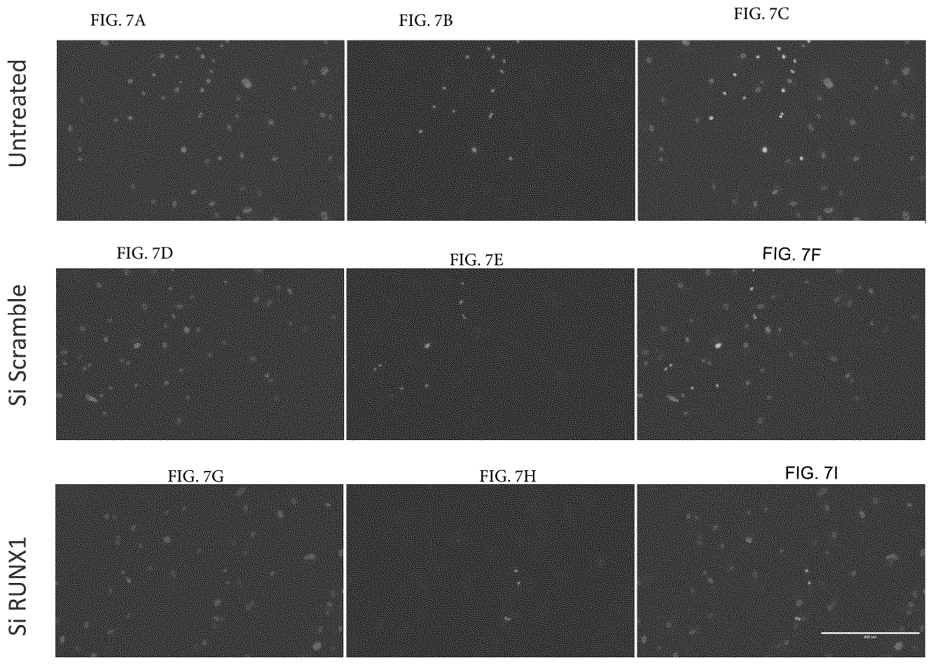

SiRNA knocked down RUNX1 expression in C-PVR cells (FIG. 6). A significant reduction in gene expression of RUNX1 in C-PVR was observed 48 hours after transfection with siRNUNXI compared to siScramble. Ki67 staining 48 hours post siRNA knockdown of RUNX1 (FIG. 7G) compared to scramble (FIG. 7D and 7F) and untreated controls (FIG. 7A-7C) showed a significant reduction in cell number and proliferative capacity of C-PVR. cells.

SIRNA

C-PVR cells (e.g., case S) were grown in a 48 well plate for 24 hours and transfected with siRNUNX1 or siScramble using Darmafect (Dharmacon). 48 hours post-transfection, cells were washed and RNA was collected and PCR was performed (FIG. 6).

Ki67 Staining of siRNA

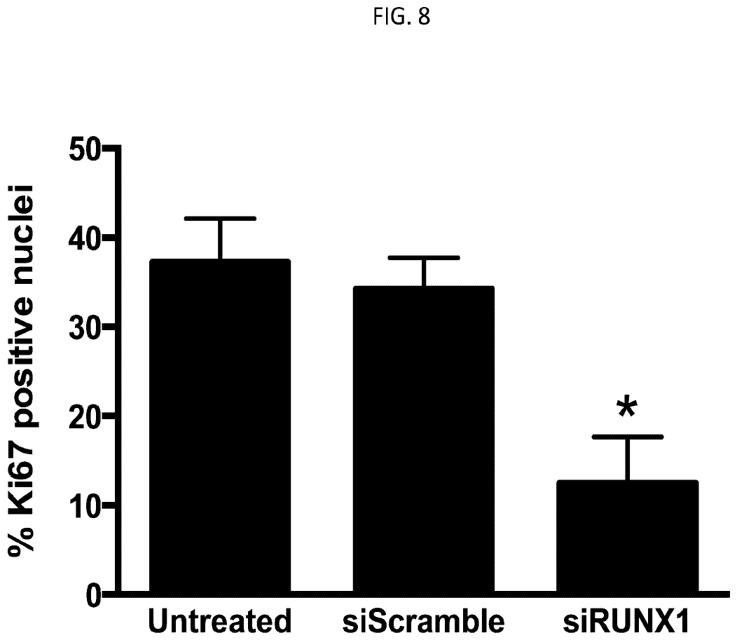

C-PVR (e.g., case 5) cells were transfected with the siRUNX1 or siScramble for 48 hours and washed and fixed with 4% paraformaldehyde for 10 minutes at room temperature. The cells were washed with PBS and blocked for an hour in (10% goat serum in PBS) incubated with rabbit anti-Ki67 antibody (1:50; Novus Biologicals) overnight at 4 degrees. Cells were incubated with secondary antibody goat anti-rabbit 594 (1:300; Life Technologies) for 2 hours along with DAPI, then were washed and imaged (FIG. 7A-7I; quantitation depicted in FIG. 8).

Treatment with RUNX1 Inhibitor

C-PVR cells (e.g., case 5) were cultured in 48 well plates for 24, followed by treatment with 150 μM RUNX1 inhibitor (Ro5-3335, Calbiochem), or vehicle only for a period of 48 hours. The cells were washed and fixed with 4% paraformaldehyde for 10 minutes at room temperature. The cells were washed with PBS and blocked for an hour in (10% goat serum in PBS) incubated with rabbit anti-Ki67 antibody (1:50; Novus Biologicals) overnight at 4 degrees. Cells were incubated with secondary antibody goat anti-rabbit 594 (1:300; Life Technologies) for 2 hours along with DAPI, washed, imaged and quantified using Image J (FIG. 9A and 9B).

Ki67 Staining of Treatment with RUNX1 Inhibitor

C-PVR cells (e.g., case 5) were cultured in 48 well plates for 24. followed by treatment with 150pM RUNX1 inhibitor (Ro5-3335, Calbiochem), or vehicle only for a period of 48 hours. The cells were washed and fixed with 4% paraformaldehyde for 10 minutes at room temperature. The cells were washed with PBS and blocked for an hour in (10% goat serum in PBS) incubated with rabbit anti-Ki67 antibody (1:50; Novas Biologicals) overnight at 4 degrees. Cells were incubated with secondary antibody goat anti-rabbit 594 (1:300); Life Technologies) for 2 hours along with DAPI, washed and imaged.

### Example 3: Effect of TGEβ, TNFα and IL-6 on ARPE-19 Cells

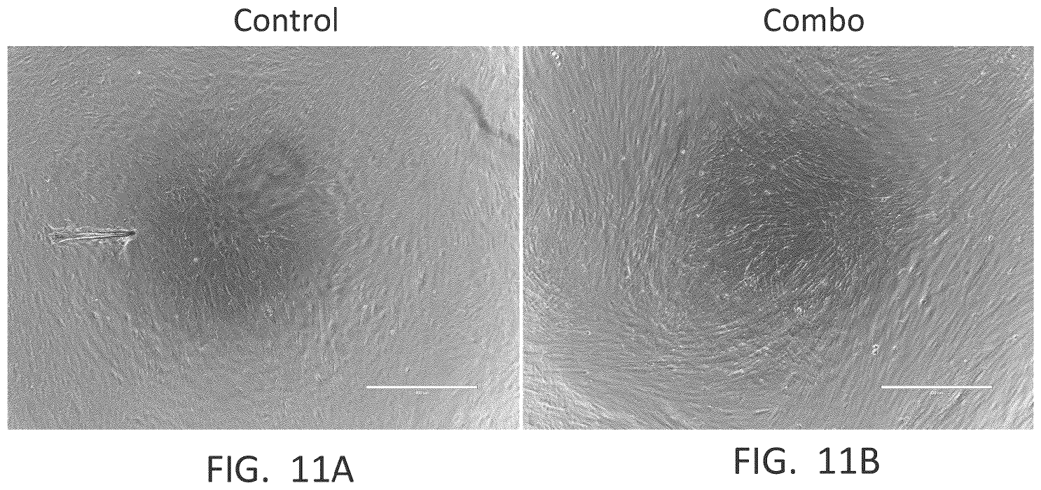

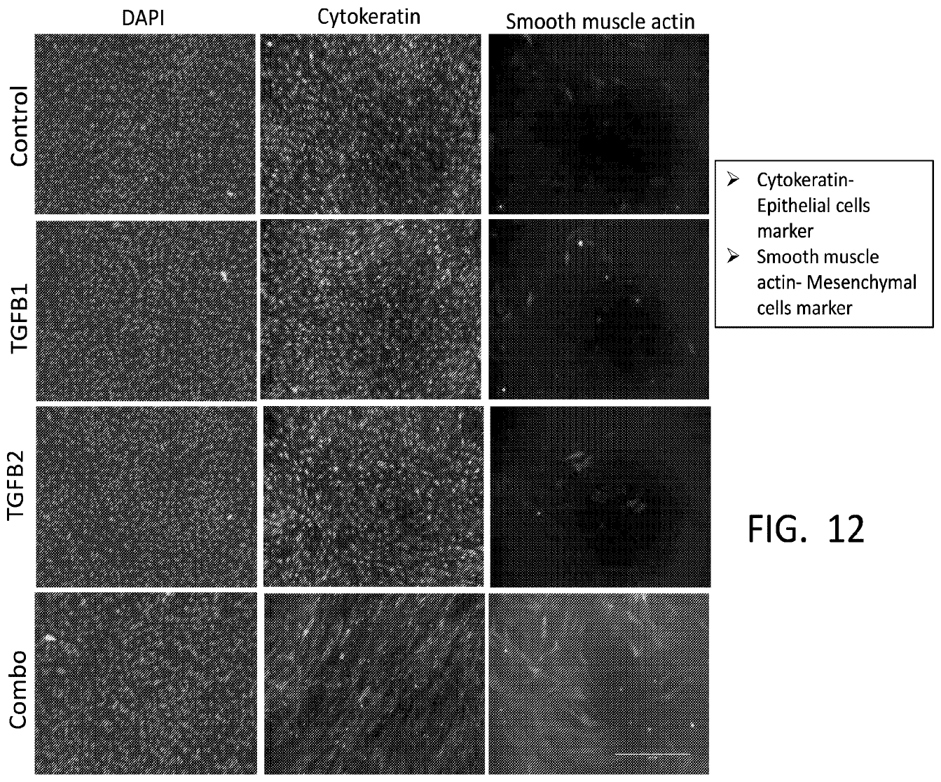

ARPE-19 cells at 7 days post treatment with TGFβ2 , TNFα, and IL-6 (all from Preprotec) showed Epithelial-Mesenchymal transition (FIG. 11B) as compared to control (FIG. 11A). ARPE-19 cells underwent EMT with combination treatment (e.g., combination of TGFβ2, TNFα, and IL-16), FIG. 12.

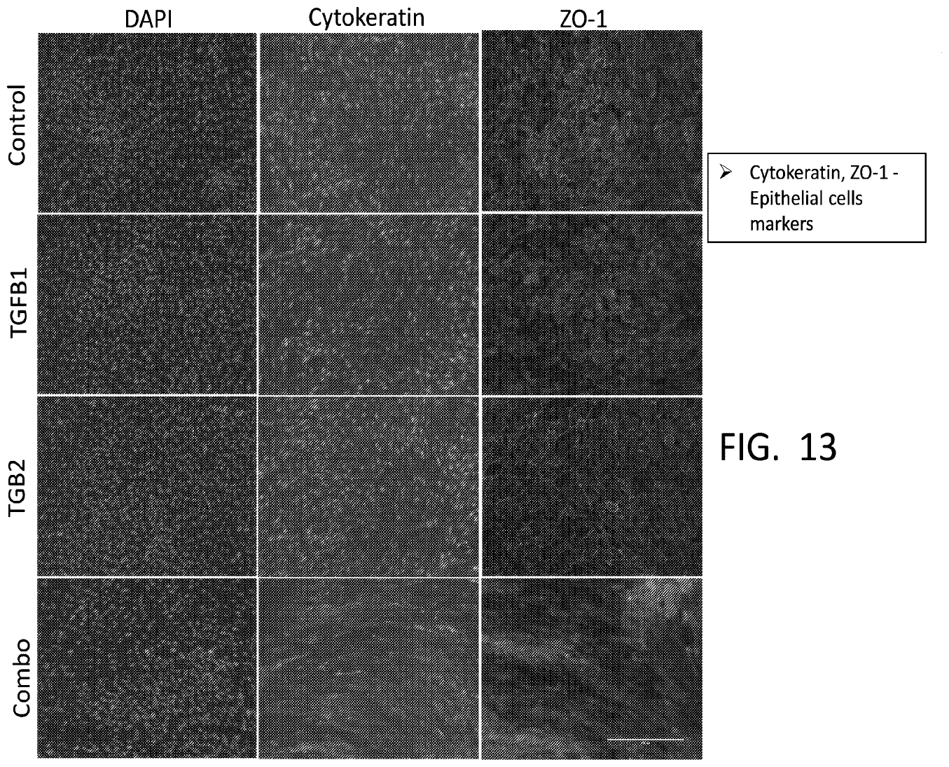

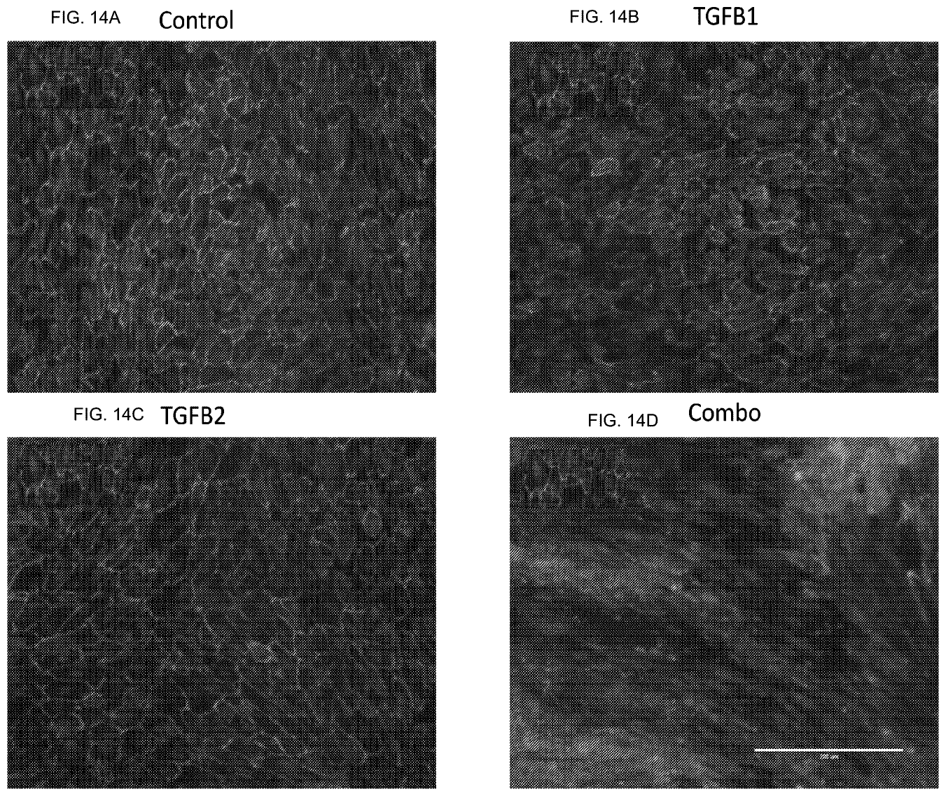

ARPE-19 cells 7 days post treatment with TGFβ1, TGFβ2, and combination of TGFβ2, TNFα and IL-6 (10 ng/ml each) showed a reduction in the epithelial markers. cytokeratin (FIG. 13, middle panel) and Zonula occludens-1 (ZO-1) (FIG. 13, right panel) in the treatments compared to controls. Losing ZO-1 organization is a marker of EMT, and therefore the data showed that the combination treatment also impacted ZO-1 distribution (FIG. 13). Additionally, ARPE-19 cells 7 days post treatment with TGFβ1, TGFβ2, and combination of TGFβ2, TNFo and IL-6 (10 ng/ml) showed a reduction in the epithelial markers Zonula occludens-1 in the treatments compared to controls (FIG. 14A-14D).

RUNX1 Expression Increases with EMT

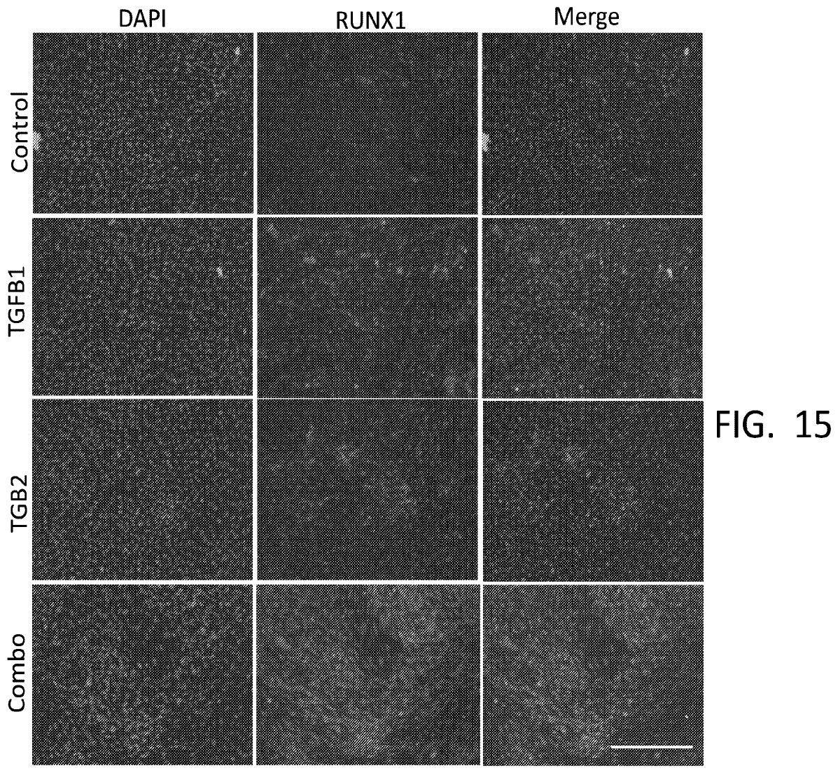

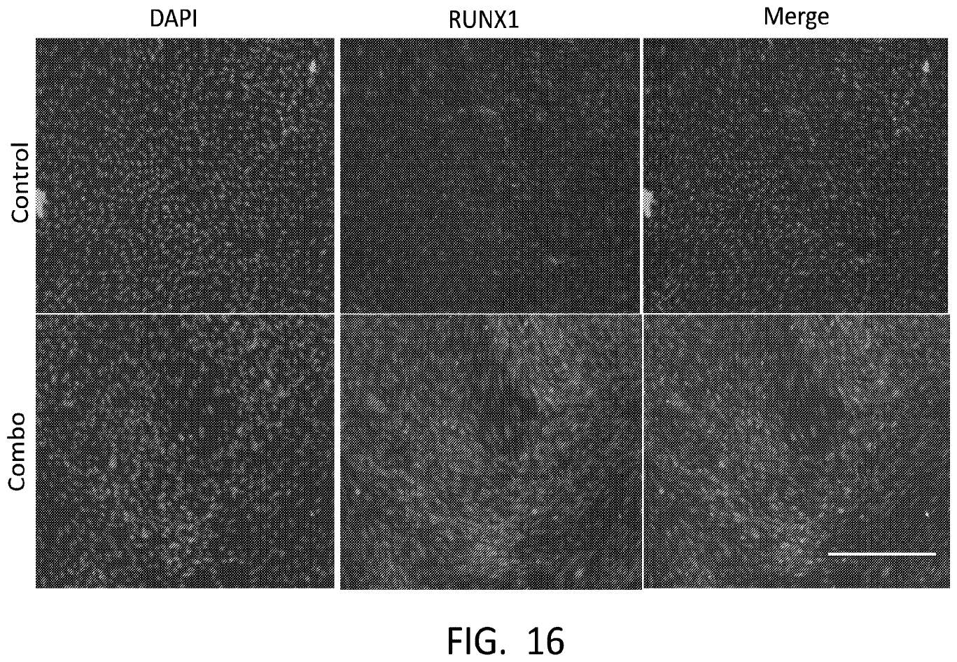

ARPE-19 cells 7 days post treatment with TGFβ1, TGFβ2, and combination of TGFβ2, TNFα and IL-6 (10 ng/ml each) showed a significant increase in the RUNX1 expression in the treatments compared to controls (FIG. 15). This indicated that RUNX1 expression increased with EMT. ARPE-19 cells 7 days post treatment with a combination of TGFβ2, TNFα and IL-6 (10 ng/ml) showed a significant increase in the RUNX1 expression in the treatments compared to controls (FIG. 16, which depicts magnified data from FIG. 13).

ARPE-19 cells 7 days post treatment with a combination of TGFβ2, TNFα and IL-6 (10 ng/ml each) showed a significant increase in the RUNX1 expression (FIG. 17, middle panel) and a reduction in the epithelial marker cytokeratin (FIG. 17, right panel) in the treatments compared to controls. This showed that RUNX1 increased expression and was also present at the 3-week time point when ZO-1 changes of distribution were present.

Cell Culture

ARPE-19 cells (ATCC) were grown to confluence and maintained in 1% for 7-10 days. Cells were treated with a combination of TGFβ2, TNFα and IL-6 (each at 10 ng/ml) for 7 days and images were taken.

Immunofluorescence

ARPE-19 cells were grown to confluence and maintained in 1% for 7-10 days. Cells were treated with TGβ1, TGFβ2, and a combination of TGFβ2, TNFα and IL-6 (10 ng/ml) for 7 days (combo). Cells were washed and fixed with 4% paraformaldehyde for 10 minutes at room temperature. Cells were incubated with primary antibodies, mouse anti-cytokeratin (1:250; Abcam) or smooth muscle actin (1:50; DAKO) overnight at 4° C. The following day, cells were incubated with secondary antibody goat anti-mouse 488 (1:300; Life Technologies) for 2 hours at room temperature and DAPI. Cells were washed with PBS and imaged (FIG. 12).

ARPE-19 cells were grown to confluence and maintained in 1% for 3 weeks. Cells were treated with TGβ1, TGFβ2, and a combination of TGFβ2, TNFα and IL-6 (10 ng/ml) for 7 days. Cells were washed and fixed with 4% paraformaldehyde for 10 minutes at room temperature. Cells were incubated with primary antibodies, mouse anti-cytokeratin (1:250; Abcam) or rabbit anti ZO1 (1:250; Life Technologies) overnight at 4° C. The following day, cells were incubated with secondary antibody goat anti-mouse 488 and goat anti- rabbit 594 (1:300; Life Technologies) for 2 hours at room temperature and DAPI. Cells were washed with PBS and imaged.

ARPE-19 cells were grown to confluence and maintained in 1% for 3 weeks. Cells were treated with TGβ1, TGFβ2, and a combination of TGFβ2, TNFα and IL-6 (10 ng/ml) for 7 days. Cells were washed and fixed with 4% paraformaldehyde for 10 minutes at room temperature. Cells were incubated with primary antibody rabbit anti ZOI (1:250; Life Technologies) overnight at 4° C. The following day, cells were incubated with secondary antibody goat anti-rabbit 594 (1:300; Life Technologies) for 2 hours at room temperature and DAPI. Cells were washed with PBS and imaged (FIG. 14A-14D).

ARPE-19 cells were grown to confluence and maintained in 1% for 7-10 days, Cells were treated with TGβ1, TGFβ2, and a combination of TGFβ2, TNFα and IL-6 (10) ng/ml) for 7 days. Cells were washed and fixed with 4% paraformaldehyde for 10 minutes at room temperature. Cells were incubated with primary antibody rabbit anti-RUNX1 (1:100; LifeSpan Biosciences) overnight at 4° C. The following day, cells were incubated with secondary antibody goat anti-mouse 594 (1:300; Life Technologies) for 2 hours at room temperature and DAPI. Cells were washed with PBS and imaged (FIG. 15).

ARPE-19 cells were grown to confluence and maintained in 1% for 7-10 days. Cells were treated with TGβ1, TGFβ2, and a combination of TGFβ2, TNFα and IL-6 (10 ng/ml) for 7 days. Cells were washed and fixed with 4% paraformaldehyde for 10 minutes at room temperature. Cells were incubated with primary antibody rabbit anti-RUNX1 (1:100; LifeSpan Biosciences) overnight at 4° C. The following day, cells were incubated with secondary antibody goat anti-rabbit 594 (1:300; Life Technologies) for 2 hours at room temperature and DAPI. Cells were washed with PBS and imaged (FIG. 16).

ARPE-19 cells were grown to confluence and maintained in 1% for 7-10 days. Cells were treated with a combination of TGFβ2, TNFα and IL-6 (10 ng/ml) for 7 days. Cells were washed and fixed with 4% paraformaldehyde for 10 minutes at room temperature. Cells were incubated with primary antibodies rabbit anti-RUNX1 (1:100; LifeSpan Biosciences) and mouse anti-cytokeratin (1:250; Abcam) overnight at 4° C. The following day, cells were incubated with secondary antibodies goat anti-mouse 488 and goat anti-rabbit 594 (1:300; Life Technologies) for 2 hours at room temperature and DAPI. Cells were washed with PBS and imaged (FIG. 17).

### Example 4: RUNX1 Expression and Effect of RUNX1 Inhibitor

RUNX1 expression is common in membranes from different donors with PVR

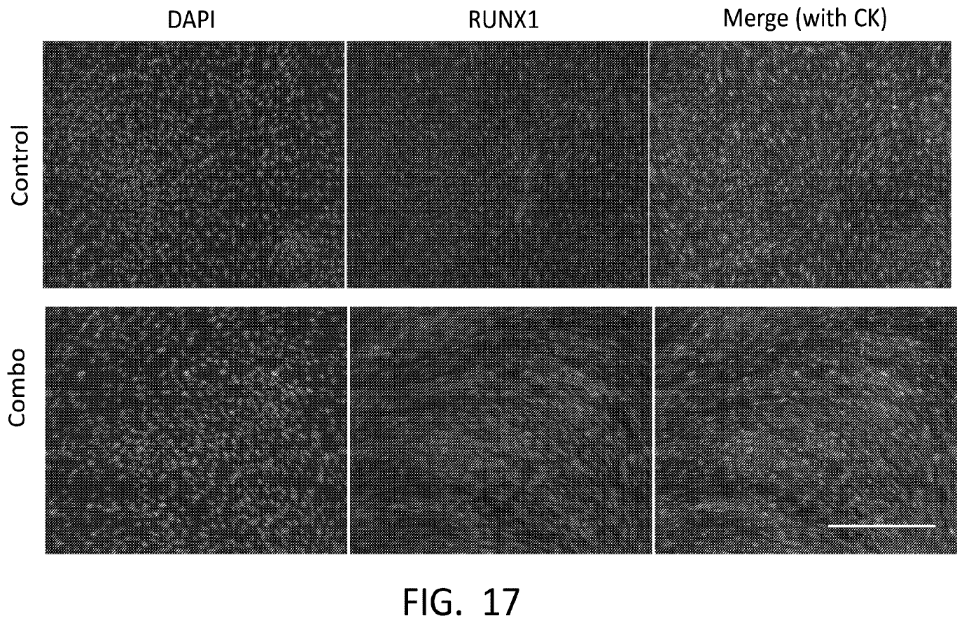

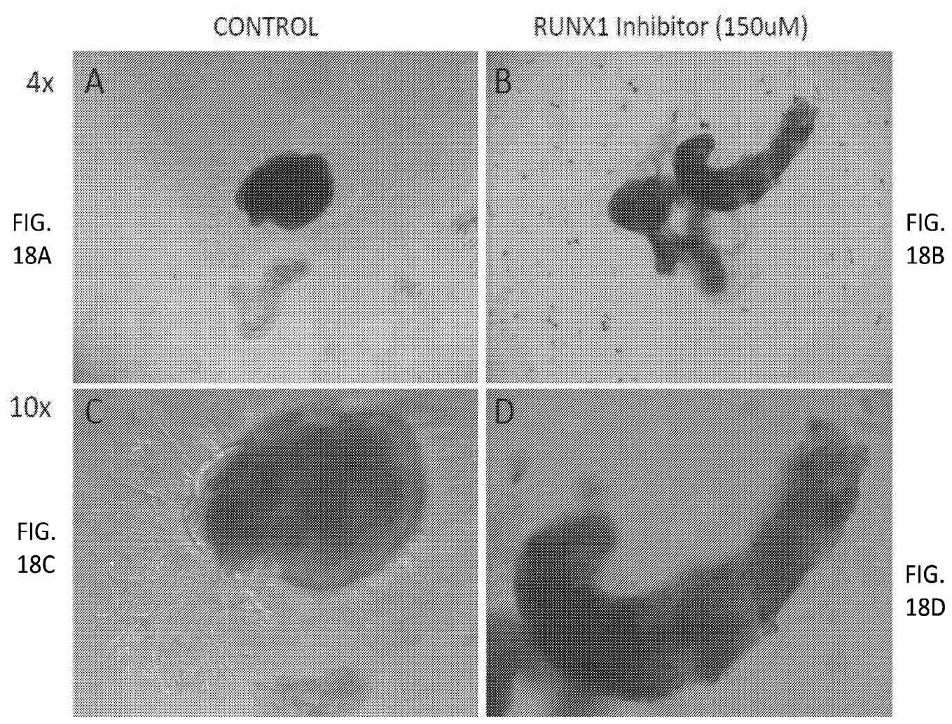

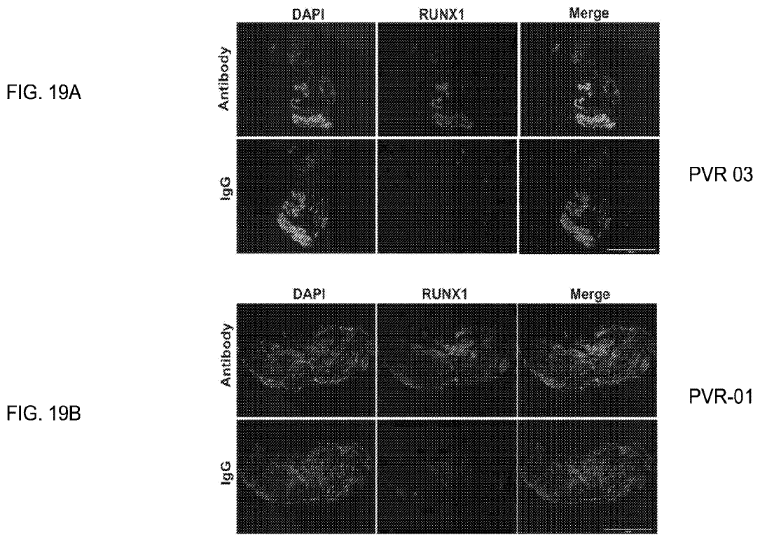

RUNX1 expression was a common feature in membranes from different donors with PVR (FIG. 19A and 19B). Freshly obtained donor human PVR membrane (Case 1 “PVR 01” and 3 “PVR 03”) were fixed in 10% formalin overnight and sectioned (6 μm). Serial sections were deparaffinized in 100% xylene, rehydrated in a series of ethanol steps and washed in phosphate buffered saline (PBS). Heat-induced epitope retrieval was performed in boiling citrate buffer (pH 6). The slides were blocked and incubated with primary antibody rabbit anti-RUNX1 (1:100; LifeSpan Biosciences) overnight at 4° C.′, followed by incubation with secondary goat anti rabbit Alexa Fluor 594 (1:300 Life Technologies) and DAPI. The slides were then washed and imaged. These data indicate common RUNX1 expression (e.g., level of RUNX1) among different donors with PVR.

The proliferation marker. Ki67. has a similar pattern for RUNX1

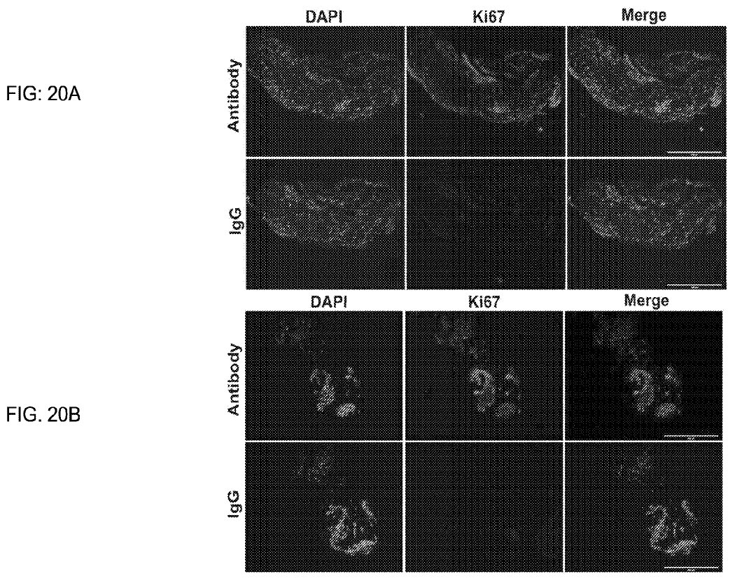

The population of cells expressing RUNX1 within PVR membranes actively proliferated, as demonstrated by their expression of Ki67 (FIG. 20A and 20B). Freshly obtained donor human PVR membrane was fixed in 10% formalin overnight and sectioned (6 μm). Serial sections were deparaffinized in 100% xylene, rehydrated in a series of ethanol steps and washed in PBS. Heat-induced epitope retrieval was performed in boiling citrate buffer (pH 6). The slides were blocked and incubated with primary antibody rabbit anti-Ki67 (1:100; Novus Biologicals) overnight at 4° C.followed by incubation with secondary goat anti rabbit Alexa Fluor 594 (1:300 Life Technologies) and DAPI. Slides were washed and imaged. For RUNX1, the slides were blocked and incubated with primary antibody rabbit anti-RUNX1 (1:100; LifeSpan Biosciences) overnight at 4° C.followed by incubation with secondary goat anti rabbit Alexa Fluor 594 (1:300 Life Technologies) and DAPI. Slides were washed and imaged. These data demonstrate that cells expressing RUNX1 within the PVR membrane were actively proliferating. This was demonstrated by the expression of Ki67.

RUNX1 Protein Expression Levels are Increased in Growth Factor-Induced EMT

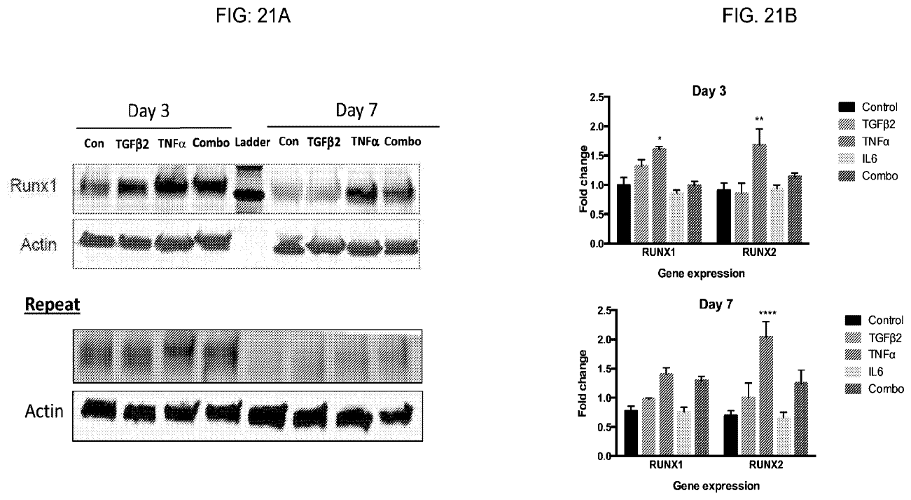

RUNX1 protein expression levels were increased in growth factor induced EMT (FIG. 21A and 21B). Increased levels of RUNX1 protein was observed upon treatment with growth factors TGFβ2, TNFα and combination treatment, including TGFβ2, TNFα and IL-6 at 3 and 7 days post treatment. Increase in RUNX1 and RUNX2 RNA expression was also observed upon treatment with growth factors at 3 and 7 days post treatment. ARPE-19 cells were seeded into 6-well pates at a density of 100,000 cells in complete media. At confluence cells were treated with growth factors TGFβ2. TNFα and a combination treatment of TGFβ2, TNFα and IL-6. Cells were washed with ice-cold PBS, lysed and collected at 3 and 7 day time points, and immunoblotted for RUNX1 (1:200, SantaCruz Biotechnology, Dallas, Texas) and B-Actin (1:1000, Cell Signaling Technology, Danvers. MA) as a loading control. ARPE-19 cells were also seeded into 6-well pates at a density of 100,000 cells, in complete media. At confluence, cells were treated with growth factors TGFβ2, TNFα and a combination treatment of TGFβ2, TNFα and IL-6. Cells were washed with PBS, lysed and collected. Quantitative RT-PCR of mRNA expression was performed and values were reported as relative fold change in expression level. These data indicate that an increase in RUNX1 protein level, and RUNX1 RNA was observed with growth factor treatment.

RUNX1 Inhibition Resulted in Inhibition of EMT

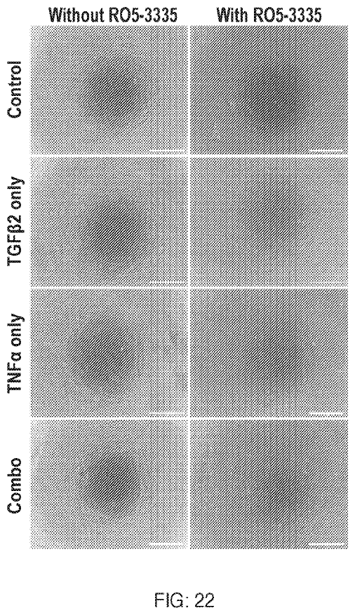

RUNX1 inhibition, using Ro5-3335, resulted in inhibition of EMT. ARPE-19 cells were grown to confluence and maintained in 1% serum for 7-10 days (FIG. 22). Cells were treated with growth factors TGFβ2, TNFα and IL-6 (10 ng/ml each) for 3 days, with 150 μM RUNX1 inhibitor (Ro5-3335, Calbiochem) or without (e.g., vehicle). The results demonstrated no (or reduced) EMT compared to vehicle treated cells. EMT was determined by morphological changes including cell shape.

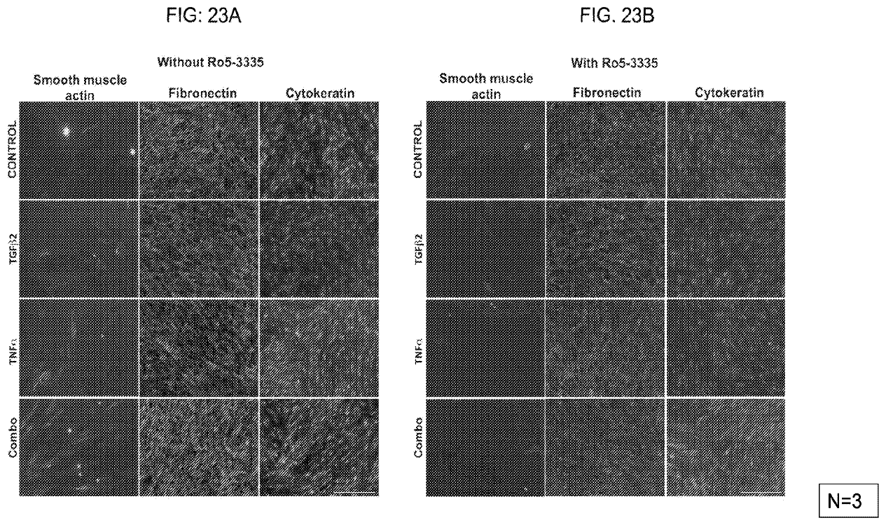

Furthermore, treatment with RUNX1 small molecule inhibitor, Ro5-335, in ARPE-19 cells prevented EMT. This was assessed by the reduction in the epithelial marker, cytokeratin, and an increase in the mesenchymal markers such as fibronectin, and smooth muscle actin (FIG. 23A and 23B). ARPE-19 cells were grown to confluence and maintained in 1% for 7-10 days. Cells were treated with TGFβ2, TNFα, and a combination of TGFβ2, TNFα and IL-6 (10 ng/ml each) for 3 days with or without 150 uM RUNX1 inhibitor (Ro5-3335, Calbiochem) or vehicle. Cells were washed and fixed with 4% paraformaldehyde for 10 minutes at room temperature. Cells were incubated with primary antibodies, mouse anti-cytokeratin (1:250; Abcam), or rabbit anti-fibronectin (1:500, Sigma) or smooth muscle actin (1:50; DAKO) overnight at 4° C. The following day, cells were incubated with secondary antibody goat anti-mouse 488 or goat anti-rabbit 594 (1:300; Life Technologies) respectively for 2 hours at room temperature and DAPI. Cells were washed with PBS and imaged. These data showed that inhibition of RUNX1 (e.g., with a small molecule), prevented EMT.

Growth Factors Induced EMT in C-PVR Cells Derived from Human Proliferative Vitreoretinopathy Membranes

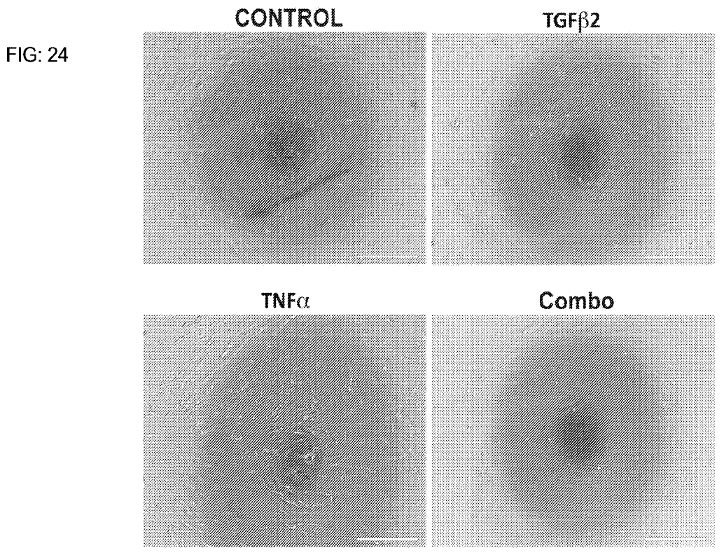

Growth factors, e.g., TNFα, TGFβ2 and IL-6 induced EMT in C-PVR cells derived from human proliferative vitreoretinopathy membranes (FIG. 24). EMT was assessed by morphological changes including cell shape, such as elongated, fibroblast-like shaped cells in the combination treatment (e.g., TNFα, TGFβ2 and IL-6). C-PVR cells were grown to confluence. Cells were treated with a combination of TGFβ2, TNFα and a combination of TGFβ2, TNFα and IL-6 (10 ng/ml each) for 7 days. These data demonstrate that growth factors induced EMT in C-PVR cells.

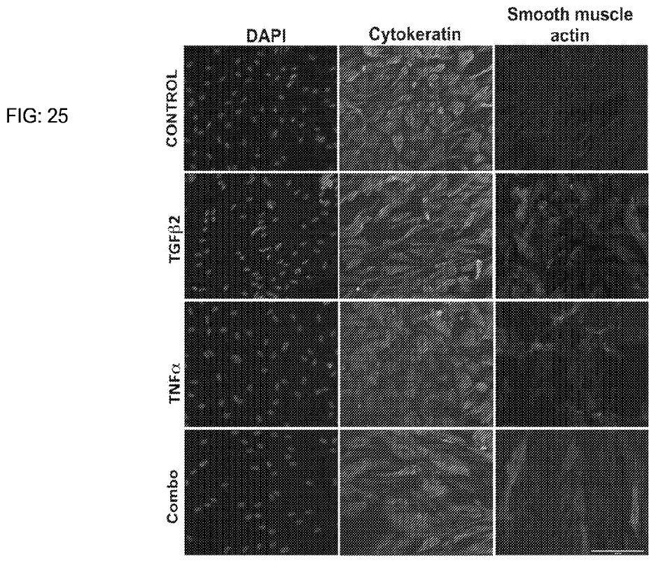

Furthermore, growth factors (e.g., TGFβ2, TNFα, and combination with TGFβ2, TNFα, and IL-6) induced EMT in C-PVR cells from human proliferative vitreoretinopathy (FIG. 25). C-PVR cells were grown to confluence. Cells were treated with TGFβ2, TNFα and a combination of TGFβ2, TNFα and IL-6 (10 ng/ml each) for 7 days. Cells were washed and fixed with 4% paraformaldehyde for 10 minutes at room temperature. Cells were incubated with primary antibodies, mouse anti-cytokeratin (1:250; Abcam) or smooth muscle actin (1:50; DAKO) overnight at 4° C. The following day, cells were incubated with secondary antibody goat anti-mouse 488 (1:300; Life Technologies) for 2 hours at room temperature and DAPI. Cells were washed with PBS and imaged. These data demonstrated that growth factors (e.g., TGFβ2, TNFα, and combination with TGFβ2, TNFα, and IL-6) induced EMT in C-PVR cells from human proliferative vitreoretinopathy (e.g., caused changes in cytokeratin and a-smooth muscle actin).

RUNX1 Expression Levels were Increased in Growth Factor-Induced EMT

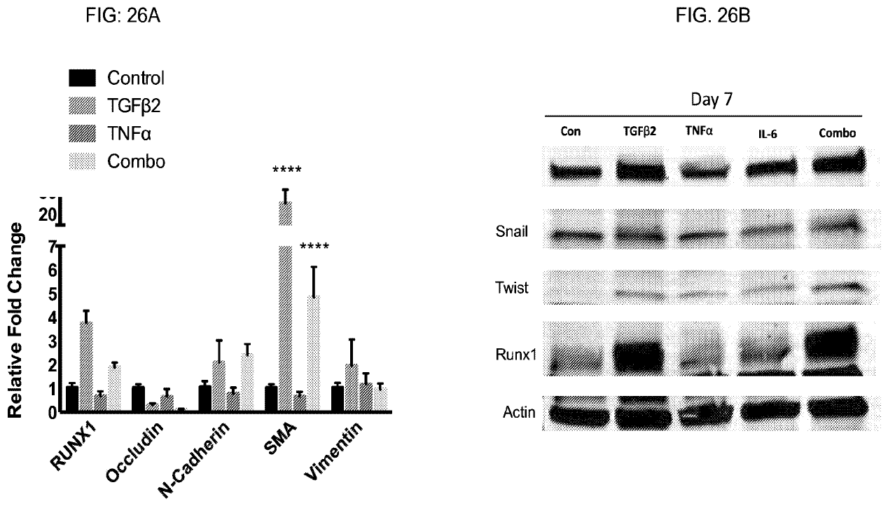

An increase in RUNX1 expression (e.g., RNA) and RUNX1 protein was observed in growth-factor induced EMT ((FIG. 26A and 26B). C-PVR cells were seeded into 12-well pates. At confluence, cells were treated with growth factors TGFβ2, TNFα and a combination treatment of TGFβ2, TNFα and IL-6. Cells were washed with PBS, lysed and collected. Quantitative RT-PCR of mRNA expression was performed and values were reported as relative fold change in expression level. C-PVR cells were also seeded into 6-well pates. At confluence, cells were treated with growth factors TGFβ2, TNFα and a combination treatment of TGFβ2, TNFα and IL-6. Cells were washed with ice-cold PBS, lysed and collected at 7 day time point and immunoblotted for RUNX1 (1:200, SantaCruz Biotechnology, Dallas, Texas), N-Cadherin (1:500, Santa Cruz Biotechnology), Snail (1: 500, Abcam), Twist (Abcam) and Beta-Actin (1:1000, Cell Signaling Technology, Danvers, MA) as a loading control. These data demonstrate that growth factors (e.g., TGFβ2 and combination—TGFβ2, TNFα, and IL-6) caused changes in EMT markers in C-PVR cells.

RUNX1 Inhibition was More Effective at Inhibiting Growth of ARPE-19 Cells and C-PVR Cells, as Compared to Methotrexate

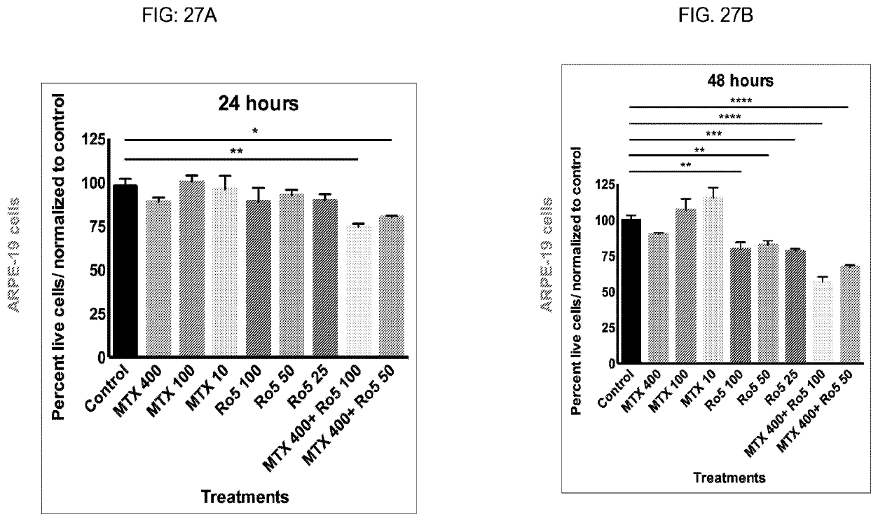

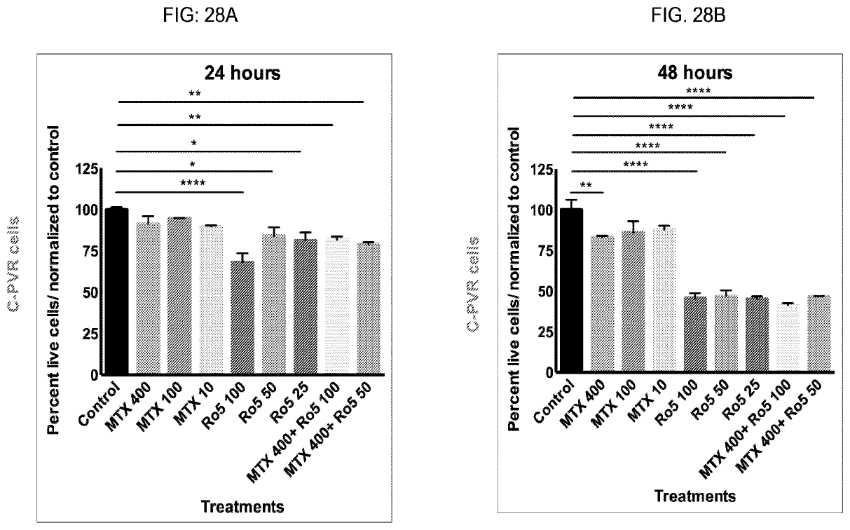

RUNX1 inhibition was more efficacious at inhibiting growth of ARPE-19 cells, as compared to treatment with methotrexate (FIG. 27A and 27B). Furthermore, the data reported that RUNX1 inhibition could be used as an adjuvant in combination to treatments (e.g., such as methotrexate). ARPE 19 cells were cultured in 96 well plates for 8 hours, followed by treatment with RUNX1 inhibitor (Ro5-3335, Calbiochem) or Methotrexate (Sigma) in growth media or vehicle only for a period of 24 or 48 hours. The cells were washed and the CyQuant Direct nucleic acid stain was added and incubated at 37° C. in the incubator. Fluorescence read outs were measured at wavelengths as instructed in the protocol. These data demonstrated the comparison of efficacy of methotrexate and RUNX1 inhibitor, and indicated that RUNX1 inhibition was more effective. Similar results were also observed in C-PVR cells. For example, it was shown that RUNX1 inhibition was more efficacious than treatment with methotrexate at inhibiting growth of C-PVR cells (FIG. 28A and FIG. 28B).

RUNX1 Knockdown Inhibited EMT in ARPE-19 Cells and Partially Inhibited EMT in C-PVR Cells

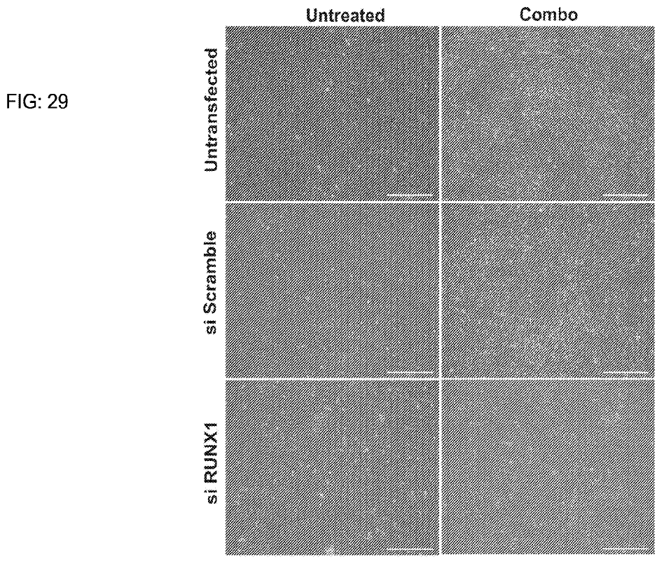

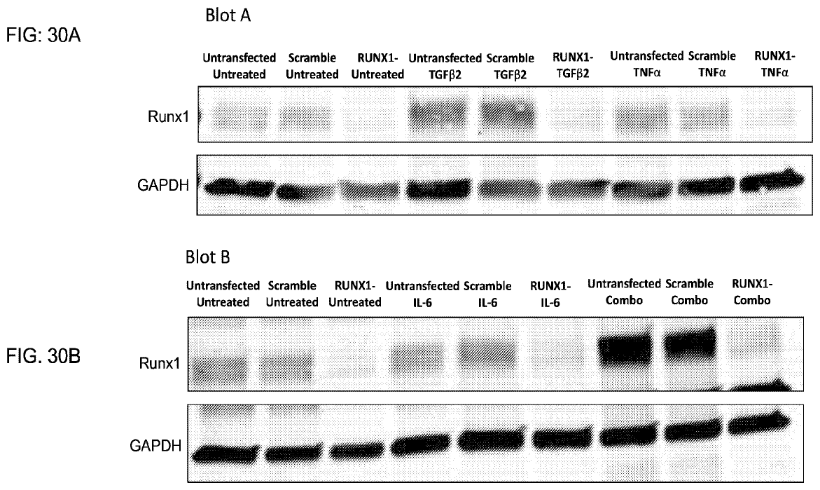

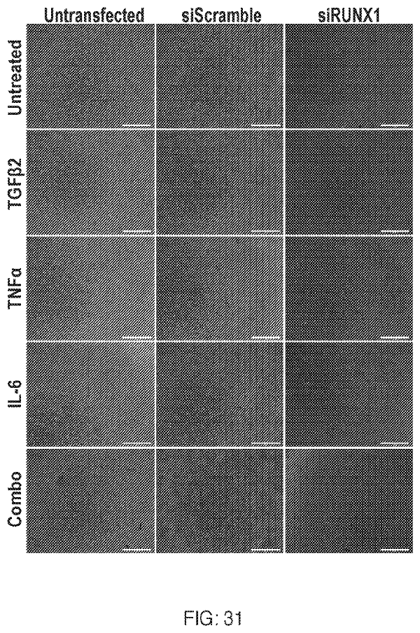

RUNX1 inhibition using siRNA resulted in the inhibition of EMT (FIG. 29). ARPE-19 cells were grown to 60-70% confluence. Cells were transfected with siScramble or siRUNX1overnight in and treated with growth factors TGFβ2, TNFα and IL-6 (10 ng/ ml) for 2 days. Bright field images were taken using the EVOS microscope. The data demonstrated that RUNX1 knockdown inhibited EMT in ARPE-19 cells. Similarly, RUNX1 inhibition using siRNA resulted in inhibition of EMT in C-PVR cells (FIG. 31). C-PVR cells were grown to 60-70% confluence. Cells were transfected with siScramble or siRUNX1 overnight in and treated with growth factors TGFβ2, TNFα and IL-6 (10 ng/ml each) for 2 days. Bright field images were taken using the EVOS microscope, and showed inhibition of EMT. EMT was determined by morphological changes (e.g., cell shape).

Growth Factor Triggered RUNX1 Induction was Inhibited Using siRNA

Growth factor triggered RUNX1 induction was inhibited using siRNA. ARPE˜19 cells were grown to 60-70% confluence. Cells were transfected with siScramble or siRUNX1 overnight in and treated with growth factors TGFβ2. TNFα and IL-6 (10 ng/ ml) for 2 days.

Cells were washed with ice-cold PBS, lysed and collected and immunoblotted for RUNX1 (1:200, SantaCruz Biotechnology, Dallas, Texas), and glyceraldehyde 3-phosphate dehydrogenase (GAPDH) (1:1000, SantaCruz Biotechnology) as a loading control. These data show that each of these growth factors (e.g., TGFβ2, TNFα and IL-6) induced protein expression of RUNX1 and that such induction was stronger when the growth factors were combined. Furthermore, the data also showed that siRNA treatment efficient reduced the induction of RUNX1 triggered by each of these growth factors alone or in combination. This data demonstrated that RUNX1 inhibition can be used to limit the effect of these growths factors as it relates to PVR.

Significant Reduction of Human PVR was Observed in an Explant Model After Treatment with RUNX1 Inhibitor

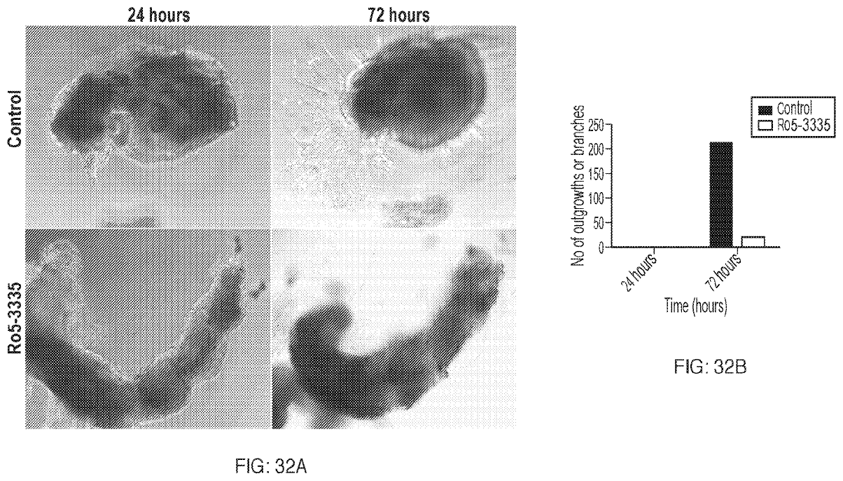

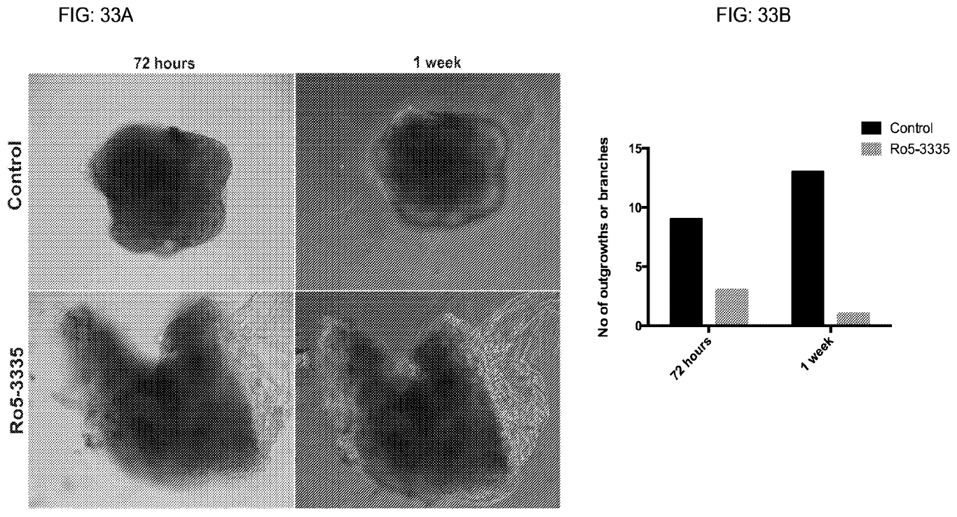

After treatment with a RUNX1 inhibitor, RoS-3335, a significant reduction of human PVR in an explant model was observed (FIG. 32A-32B and FIG. 33A-33B). This result was observed from two different donors. PVR membranes were freshly obtained from patients and placed in growth factor-reduced Matrigel (BD Biosciences) seeded in 24 well plates. 30 μl of matrigel were used to coat the bottom of 24 well plates without touching the edges of the well. After seeding the PVR membrane, explant plates were incubated in a 37° C. cell incubator without medium for 10 minutes in order for the Matrigel to solidify. 500 μl of medium was added to each well and incubated at 37° C. with 5% CO₂. The explants were treated with RUNX1 inhibitor (Ro5-3335). Phase contrast photos of individual explants were taken daily.

Synergistic Effect was Observed with the Combination of RUNX1 Inhibition and Methotrexate

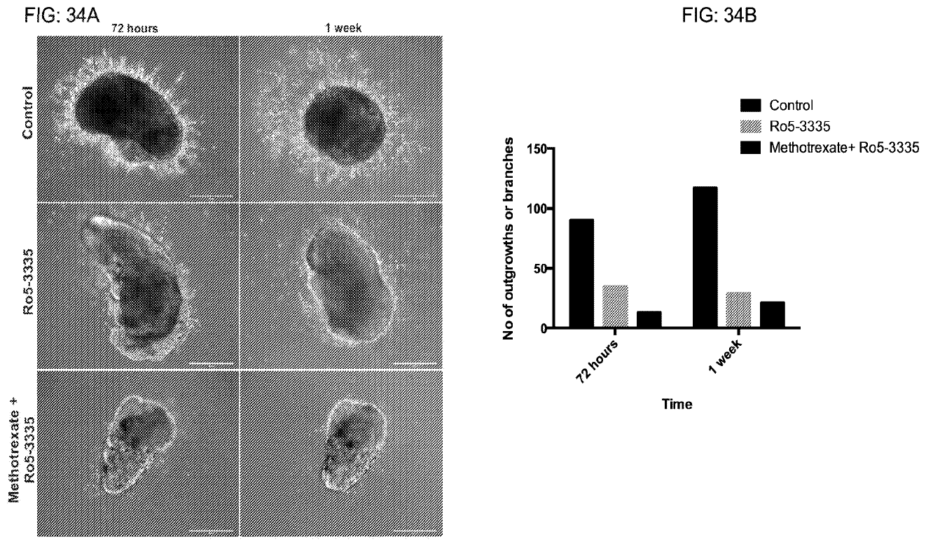

Explant data reveled the reduction of the growth of PVR membranes in an explant model. Furthermore, a synergistic effect was observed, when RUNX1 inhibition (e.g., with Ro5-3335) was combined with methotrexate (FIG. 34A and 34B). PVR membranes were freshly obtained from patients, placed in growth factor-reduced Matrigel (BD Biosciences), and seeded in 24 well plates. 30 μl of matrigel was used to coat the bottom of 24 well plates without touching the edges of the well. After seeding, the PVR membrane explant plates were incubated in a 37° C. cell incubator without medium for 10 minutes in order for the Matrigel to solidify. 500 μl of medium was added to each well and incubated at 37° C. with 5% CO₂. The explants were treated with RUNX1 inhibitor (Ro5-3335) or a combination of Ro5-3335 and methotrexate. Phase contrast photos of individual explants were taken daily.

Lenlidomide Inhibited Proliferation in C-PVR Cells

C-PVR cells were treated with lenalidomide, and it was found that lenalidomide treatment significantly reduced proliferation of C-PVR cells. Lenalidomide treatment alone or in combination with a RUNX1 inhibitor may be used for the treatment of PVR.

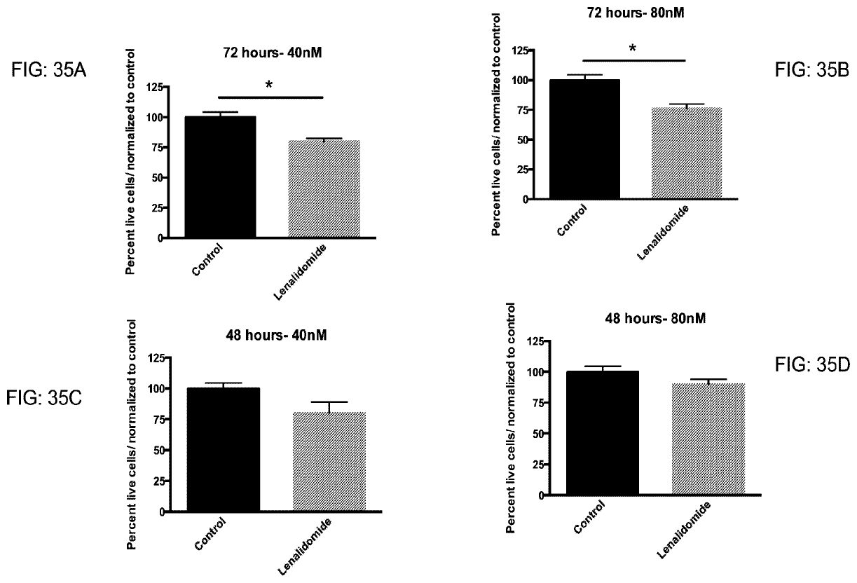

Lenlidomide inhibited proliferation in C-PVR cells at 48 and 72 hours, with 40 and 80 nM (FIG. 35A-35D). A CyQuant Cell Proliferation Assay was performed 48 and 72 hours post-treatment, with lenalidomide at 40 nM, and 80 nM. The data were compared to vehicle treated, and showed a significant reduction in percent live cells at 72 hours. C-PVR cells were cultured in 96 well plates for 8 hours, followed by treatment with lenalidomide (Sigma-Aldrich) in growth media or vehicle only for a period of 48 or 72 hours. The cells were washed, and CyQuant Direct and nucleic acid stain was added and incubated at 37° C. Fluorescence read outs were measured at wavelengths as instructed in the protocol.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A method for preventing or reducing proliferation or migration of retinal pigment epithelial (RPE) cells in a subject who comprises a retinal hole or retinal tear, the method comprising administering to the subject a composition comprising runt-related transcription factor 1 (RUNX1) inhibitor.

2.-28. (canceled)

29. A method for diagnosing aberrant epithelial to mesenchymal transition (EMT) of retinal cells in a subject or a method for monitoring whether a disease that comprises PVR is progressing in a subject who has been diagnosed with the disease, comprising periodically determining the level of RUNX1 protein or mRNA in a said subject, or a method for diagnosing a PVR in a subject, or a method for identifying whether a therapy has reduced or ameliorated a disease that comprises PVR, pathologic ocular fibrosis, or pathologic ocular proliferation in a subject,
wherein said method of diagnosing EMT comprises the steps of:
(a) providing a test sample from said subject
(b) assaying the level of RUNX1 protein or mRNA in the test sample; and
(c) diagnosing the subject as having aberrant EMT of retinal cells if the level of RUNX1 protein or mRNA is elevated in the test sample compared to a normal control,
wherein said monitoring comprises the steps of:
(1) identifying the disease as worsening if the level of RUNX1 protein or mRNA increases over time;
(2) identifying the disease as improving if the level of RUNX1 protein or mRNA decreases over time;
(3) identifying the disease as neither worsening or improving if the level of RUNX1 protein or mRNA remains the same or about the same over time,
wherein determining the level of RUNX1 protein or mRNA comprises
(a) providing a test sample from said subject; and
(b) assaying the level of RUNX1 protein or mRNA in the test sample;
wherein said diagnosing a PVR comprises the steps of:
(a) providing a test sample from said subject
(b) assaying the level of RUNX1 protein or mRNA in the test sample; and
(c) diagnosing the subject as having aberrant PVR if the level of RUNX1 protein or mRNA is elevated in the test sample compared to a normal control;
wherein said identifying whether a therapy has reduced or ameliorated a disease that comprises PVR, pathologic ocular fibrosis, or pathological ocular proliferation in a subject comprises:
(a) providing a pre-therapy test sample from said subject (b) assaying the pre-therapy level of RUNX1 protein or mRNA in the pre-therapy test sample;
(c) administering the therapy to the subject;
(d) providing a post-therapy test sample from said subject;
(e) assaying the post-therapy level of RUNX1 protein or mRNA in the post-therapy test sample; and
(f) identifying the therapy as having reduced or ameliorated said disease if the level of RUNX1 protein or mRNA in the post-therapy test sample is lower than the level of RUNX1 protein or mRNA in the pre-therapy sample.

30. A method for treating or reducing the severity of PVR in a subject, said method comprising:
(a) diagnosing PVR in a subject according to the method of claim 29, and
(b) administering to said subject a RUNX1 inhibitor.

31.-38. (canceled)

39. The method of claim 29, wherein the test sample from the subject is an ocular sample.

40. The method of claim 29, wherein the test sample from the subject is an ocular sample obtained during ocular surgery.

40. The method of claim 29, wherein the subject is a human.

41. The method of claim 29, wherein the level of RUNX1 protein is measured by ELISA, Western blot analysis, immunohistochemistry, or immunofluorescence.

42. The method of claim 29, wherein the level of RUNX1 mRNA is measured by Q-RT-PCR.

43. The method of claim 30, wherein the RUNX1 inhibitor comprises a small molecule.

44. The method of claim 30, wherein the small molecule comprises the structure of Formula I: 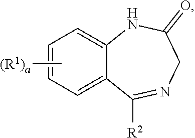
or a pharmaceutically acceptable salt or ester thereof, wherein
each R₁ is individually selected from halogen, alkyl, aryl, heteroaryl, or alkoxy,
and R2 is selected from aryl or heteroaryl, and
a is 0 to 4;
Formula III: 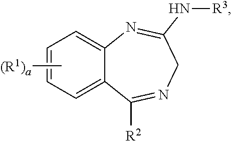
or a pharmaceutically acceptable salt or ester thereof, wherein
each R1 is individually selected from halogen, alkyl, aryl, heteroaryl, or alkoxy,
R² is selected from aryl or heteroaryl;
R³ is alkyl or aryl, and
a is 0 to 4; or
Formula V: 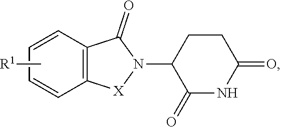
or a pharmaceutically acceptable salt or ester thereof, wherein
R¹ is H³, NH², or NHC(O)CH₃, and
X is CH₂ or C(O).

45. The method of claim 43, wherein the small molecule is Ro5-3335, Ro24-7429, or lenalidomide.

46. The method of claim 29, wherein the RUNX1 inhibitor comprises an inhibitory nucleic acid.

47. The method of claim 46, wherein the inhibitory nucleic acid is selected from the group consisting of an RNA interfering agent (RNAi), an RNA encoding a RUNX1 inhibitory protein.

48. The method of claim 29, wherein the RUNX1 inhibitor is a dominant negative CBF-Beta protein, or an RNA encoding a dominant negative CBF-Beta protein.

49. The method of claim 48, wherein the dominant negative CBF-Beta protein is CBFB-MYH11.

50. The method of claim 29, wherein the RUNX1 inhibitor is administered during or after intraocular surgery.

51. A method comprising providing a test sample from said subject, wherein the test sample is an ocular sample, and assaying levels of RUNX1 in the test sample.

52. The method of claim 51, wherein ocular sample is obtained during ocular surgery.

53. The method of claim 51, wherein the subject is a human.

54. The method of claim 51, wherein the level of RUNX1 is assayed by ELISA, Q-RT-PCR, Western blot analysis, immunohistochemistry, or immunofluorescence.


SEQUENCE LISTING (39 sequences)

SEQ ID NO: 1 - protein, 453 aa, Homo sapiens
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 2 - protein, 472 aa, Homo sapiens
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMGGASCSRQARRDPGPWARTPSWGRGRPTDRISL

SEQ ID NO: 3 - protein, 250 aa, Homo sapiens
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQEEDTAPWRC

SEQ ID NO: 4 - protein, 257 aa, Homo sapiens
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQS

SEQ ID NO: 5 - protein, 224 aa, Homo sapiens
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRSKCIHLGLVHPPGWYTLQAGILRDHVSDSLGSTFPPGGWQAPVKPKS

SEQ ID NO: 6 - protein, 188 aa, Homo sapiens
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRNSLTWPRYPHI

SEQ ID NO: 7 - protein, 242 aa, Homo sapiens
MRIPVDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPI

SEQ ID NO: 8 - protein, 480 aa, Homo sapiens
MASDSIFESFPSYPQCFMRECILGMNPSRDVHDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 9 - protein, 456 aa, Homo sapiens
MNPSRDVHDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 10 - protein, 468 aa, Homo sapiens
MPAAPRGPAQGEAAARTRSRDASTSRRFTPPSTALSPGKMSEALPLGAPDAGAALAGKLRSGDRSMVEVLADHPGELVRTDSPNFLCSVLPTHWRCNKTLPIAFKVVALGDVPDGTLVTVMAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 11 - protein, 348 aa, Homo sapiens
MAGNDENYSAELRNATAAMKNQVARFNDLRFVGRSGRGKSFTLTITVFTNPPQVATYHRAIKITVDGPREPRRHRQKLDDQTKPGSLSFSERLSELEQLRRTAMRVSPHHPAPTPNPRASLNHSTAFNPQPQSQMQDTRQIQPSPPWSYDQSYQYLGSIASPSVHPATPISPGRASGMTTLSAELSSRLSTAPDLTAFSDPRQFPALPSISDPRMHYPGAFTYSPTPVTSGIGIGMSAMGSATRYHTYLPPPYPGSSQAQGGPFQASSPSYHLYYGASAGSYQFSMVGGERSPPRILPPCTNASTGSALLNPSLPNQSDVVEAEGSHSNSPTNMAPSARLEEAVWRPY

SEQ ID NO: 12 - DNA, 7274 bp, Homo sapiens
  source 1..7274: mol_type = genomic DNA
catagagccagcgggcgcgggcgggacgggcgccccgcggccggacccagccagggcaccacgctgcccggccctgcgccgccaggcacttctttccggggctcctagggacgccagaaggaagtcaacctctgctgcttctccttggcctgcgttggaccttcctttttttgttgtttttttttgtttttcccctttcttccttttgaattaactggcttcttggctggatgttttcaacttctttcctggctgcgaacttttccccaattgttttccttttacaacagggggagaaagtgctctgtggtccgaggcgagccgtgaagttgcgtgtgcgtggcagtgtgcgtggcaggatgtgcgtgcgtgtgtaacccgagccgcccgatctgtttcgatctgcgccgcggagccctccctcaaggcccgctccacctgctgcggttacgcggcgctcgtgggtgttcgtgcctcggagcagctaaccggcgggtgctgggcgacggtggaggagtatcgtctcgctgctgcccgagtcagggctgagtcacccagctgatgtagacagtggctgccttccgaagagtgcgtgtttgcatgtgtgtgactctgcggctgctcaactcccaacaaaccagaggaccagccacaaacttaaccaacatccccaaacccgagttcacagatgtgggagagctgtagaaccctgagtgtcatcgactgggccttcttatgattgttgttttaagattagctgaagatctctgaaacgctgaattttctgcactgagcgttttgacagaattcattgagagaacagagaacatgacaagtacttctagctcagcactgctccaactactgaagctgattttcaaggctacttaaaaaaatctgcagcgtacattaatggatttctgttgtgtttaaattctccacagattgtattgtaaatattttatgaagtagagcatatgtatatatttatatatacgtgcacatacattagtagcactacctttggaagtctcagctcttgcttttcgggactgaagccagttttgcatgataaaagtggccttgttacgggagataattgtgttctgttgggactttagacaaaactcacctgcaaaaaactgacaggcattaactactggaacttccaaataatgtgtttgctgatcgttttactcttcgcataaatattttaggaagtgtatgagaattttgccttcaggaacttttctaacagccaaagacagaacttaacctctgcaagcaagattcgtggaagatagtctccactttttaatgcactaagcaatcggttgctaggagcccatcctgggtcagaggccgatccgcagaaccagaacgttttcccctcctggactgttagtaacttagtctccctcctcccctaaccacccccgcccccccccaccccccgcagtaataaaggcccctgaacgtgtatgttggtctcccgggagctgcttgctgaagatccgcgcccctgtcgccgtctggtaggagctgtttgcagggtcctaactcaatcggcttgttgtgatgcgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcaggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggcccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaactttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaaatgcttctttctactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacatacaccata

SEQ ID NO: 13 - DNA, 5967 bp, Homo sapiens
  source 1..5967: mol_type = genomic DNA
ctttgggcctcataaacaaccacagaaccacaagttgggtagcctggcagtgtcagaagtctgaacccagcatagtggtcagcaggcaggacgaatcacactgaatgcaaaccacagggtttcgcagcgtggtaaaagaaatcattgagtcccccgccttcagaagagggtgcattttcaggaggaagcgatggcttcagacagcatatttgagtcatttccttcgtacccacagtgcttcatgagagaatgcatacttggaatgaatccttctagagacgtccacgatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcaggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggcccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaactttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaaatgcttctttctactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacatacaccata

SEQ ID NO: 14 - DNA, 2722 bp, Homo sapiens
  source 1..2722: mol_type = genomic DNA
catagagccagcgggcgcgggcgggacgggcgccccgcggccggacccagccagggcaccacgctgcccggccctgcgccgccaggcacttctttccggggctcctagggacgccagaaggaagtcaacctctgctgcttctccttggcctgcgttggaccttcctttttttgttgtttttttttgtttttcccctttcttccttttgaattaactggcttcttggctggatgttttcaacttctttcctggctgcgaacttttccccaattgttttccttttacaacagggggagaaagtgctctgtggtccgaggcgagccgtgaagttgcgtgtgcgtggcagtgtgcgtggcaggatgtgcgtgcgtgtgtaacccgagccgcccgatctgtttcgatctgcgccgcggagccctccctcaaggcccgctccacctgctgcggttacgcggcgctcgtgggtgttcgtgcctcggagcagctaaccggcgggtgctgggcgacggtggaggagtatcgtctcgctgctgcccgagtcagggctgagtcacccagctgatgtagacagtggctgccttccgaagagtgcgtgtttgcatgtgtgtgactctgcggctgctcaactcccaacaaaccagaggaccagccacaaacttaaccaacatccccaaacccgagttcacagatgtgggagagctgtagaaccctgagtgtcatcgactgggccttcttatgattgttgttttaagattagctgaagatctctgaaacgctgaattttctgcactgagcgttttgacagaattcattgagagaacagagaacatgacaagtacttctagctcagcactgctccaactactgaagctgattttcaaggctacttaaaaaaatctgcagcgtacattaatggatttctgttgtgtttaaattctccacagattgtattgtaaatattttatgaagtagagcatatgtatatatttatatatacgtgcacatacattagtagcactacctttggaagtctcagctcttgcttttcgggactgaagccagttttgcatgataaaagtggccttgttacgggagataattgtgttctgttgggactttagacaaaactcacctgcaaaaaactgacaggcattaactactggaacttccaaataatgtgtttgctgatcgttttactcttcgcataaatattttaggaagtgtatgagaattttgccttcaggaacttttctaacagccaaagacagaacttaacctctgcaagcaagattcgtggaagatagtctccactttttaatgcactaagcaatcggttgctaggagcccatcctgggtcagaggccgatccgcagaaccagaacgttttcccctcctggactgttagtaacttagtctccctcctcccctaaccacccccgcccccccccaccccccgcagtaataaaggcccctgaacgtgtatgttggtctcccgggagctgcttgctgaagatccgcgcccctgtcgccgtctggtaggagctgtttgcagggtcctaactcaatcggcttgttgtgatgcgtatccccgtagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggaggaagacacagcaccctggagatgttaaggcagaagtcagttcttctgtccatccctctccccagccaggatagagctatcttttccatctcatcctcagaagagactcagaagaaagatgacagccctcagaatgcacgttatgaggaaggcagaatgtgggtctgtaattcctccgtgtcccttctccccctctgcaaaccgtcgtaacaataatagttcctaacacatgggacaattgtgaggattaaatgagttagcctgcagaaatcacttgatgcacagcacatgggaagcattgtgtgtatttattaatccttcacaaagtctttgagatatatttttatcaaatatttagcatggatcccggtacactttcaatacttaataaatggtcaatgttattctttttcactatt

SEQ ID NO: 15 - DNA, 6230 bp, Homo sapiens
  source 1..6230: mol_type = genomic DNA
agggcggctgctgccccctggcgtccgccagagcccgcggcgcgtcccgacagctccgcgcagtccgggcccgcccgggaaaggcctccacgccagtcaaccccggccccctcctgcgtccgggcccgcggcaacgcccggggacagtccctgcgcggcagcggccacagcaccctagccctgggggagggaggagcgggcgtcaggggccccgtgaggtcggcgagggacccccgcgccggggagcttaattgaggggctccgcggggaccacaaagcagcccgcggtggaacggggccgggatccggggactgggttgcagccccggggtggatgagaggccccgggaggtgcagggcccggcagggagggcgtggagggactcatggtgacgggagcagcctccgctgggagggagccccgccgggcggggtcggagttggcagcgcacgccggaccgggggcggggccctccgagctccgcgaaaatgcccgcagcaccgcggggaccggcgcagggggaagcggccgcccgcacgcgaagccgggatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcaggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggcccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaactttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaaatgcttctttctactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacatacaccata

SEQ ID NO: 16 - DNA, 1452 bp, Homo sapiens
  source 1..1452: mol_type = genomic DNA
acagttaaatttgtaatttgggttgtgtgaaaacttctttgggcctcataaacaaccacagaaccacaagttgggtagcctggcagtgtcagaagtctgaacccagcatagtggtcagcaggcaggacgaatcacactgaatgcaaaccacagggtttcgcagcgtggtaaaagaaatcattgagtcccccgccttcagaagagggtgcattttcaggaggaagcgatggcttcagacagcatatttgagtcatttccttcgtacccacagtgcttcatgagagaatgcatacttggaatgaatccttctagagacgtccacgatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggaggaagacacagcaccctggagatgttaaggcagaagtcagttcttctgtccatccctctccccagccaggatagagctatcttttccatctcatcctcagaagagactcagaagaaagatgacagccctcagaatgcacgttatgaggaaggcagaatgtgggtctgtaattcctccgtgtcccttctccccctctgcaaaccgtcgtaacaataatagttcctaacacatgggacaattgtgaggattaaatgagttagcctgcagaaatcacttgatgcacagcacatgggaagcattgtgtgtatttattaatccttcacaaagtctttgagatatatttttatcaaatatttagcatggatcccggtacactttcaatacttaataaatggtcaatgttattctttttcactatta

SEQ ID NO: 17 - DNA, 4693 bp, Homo sapiens
  source 1..4693: mol_type = genomic DNA
acagttaaatttgtaatttgggttgtgtgaaaacttctttgggcctcataaacaaccacagaaccacaagttgggtagcctggcagtgtcagaagtctgaacccagcatagtggtcagcaggcaggacgaatcacactgaatgcaaaccacagggtttcgcagcgtggtaaaagaaatcattgagtcccccgccttcagaagagggtgcattttcaggaggaagcgatggcttcagacagcatatttgagtcatttccttcgtacccacagtgcttcatgagagaatgcatacttggaatgaatccttctagagacgtccacgatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaaacagttgtgattacttcaggtttcaccagatgccttagctgctgttcagagcttcacagaatgccttctaagagcttcacaacggcagtactattgacatttggggccaaataattctttgttggggggggtctgtcctgagcatcggagaatatttagcagcatccttggcctctagatgccaatagcatccccctcaccccagttaggacaataaaaaatgtttccagacattgccaaatttcccaaggggcaaaactgtccccaggtgagaaccactgccttccagtgttcttgggtggtacagtaaaataggaaaaccaacttaagtgcttggttttcacattacttttggccattaatgccaatattttcatggtacattctgctccaaggcattgagagtagcacaggtatcttggcatcttgtttagcttgtggtttctagaagcttatccctcaagaacaaaggcacttgtcagcttttactgattgttctcagggcacccctctgcacaccggctacggtcttcacctgaaataatgcctgttctccttcaaatgttcctgctatgggtctatcctttggaatttattacataagtcttttgacttggtcatcacccaagatcagcaccttatagtccaagaaccctaggggccaactcctccagctgactggtgttcagatggtagggtgactttttaaataaaaggaatgagtaacatgtgcactccaaagtcccatatcacagttacatttgtgaatatcctggacttcaatcacccaaggagtaagagagaacattttatggcatgggagtgtgaagtagataaaaaccatgcctcatttcagaacccaagaatgattcttttctttttggagggaggacagaattccagatgagctttcacagtggcatagaacagtatgggagactgctcttcaatttggcagaactccctctgagaaatcaagcctggagcacaggcctgaaaagtgttgtcataaacctggaggcaggaggggggtggaatacataaaaggaaagtggctgcctggccgacagctttgagaacctgacactgctctgtggctgctttgccttttaatccagcccgacagctttgtgctctgcccctcttcccggccccagccccacctgggcttccttctgcgctggggacttggactctgtctgtctctctctctctatcctttgcagacagactccacaccttgtctgaaaagagttgtcatctagaggtttacttttttttttctttttcaaaaaaaattaatgtttagctgccaacctgggcgacagacagcatgcattaatctgagtcatgcaaaagctccttttaaggcagccattacctgctcaggttgtttaatcctctgggggcagtaggaggggccctgtagcgaggctgctttcatccatgtcaagctggagagcgaagacttgagctgtcatggcaaatatttagccccatcaatgggaaaacacttccaaccaaaatacctggacctcctaagccccagatttcctactcttaaaaactctctgtgaatatcaggggatcccaactccccccacacttctgctgaatcaggggacaccaggccagccaaaaggacagaagagctacagctggatgggtgaactgcgcagactgatgggtttgaataagggatctgagacaagacacaaagatgaaaaccaacatttgaatatagcatttgaatctgagcatatctagaaggcctaagatattgtattatcaaaatgtgtgtaaaaaagatagtttgaccaaatggatggtcttttgtagtatgacgttgctcatgtgtacatagaagaaggatcacaggcagcattgcagctctttcttttggatgttctcctcacaagtgaaagaactattttcccccaacagctgagaccataacatcattccagtacttatgtccatgacaaatcacggactcactctggattccagtaacttggtaccatcaccacattaccatgtagtagttggaacctactggcagagcatgtccagacattgctgtaaaacagggtgacctttcgttcattttccagtggattcacctttttgtctcagggcaaatgaagcaagaggaataatgggcagagtcatttatttgtgtccagagtattgcatcagatgacaacaaactgactgtatttgcctaatgcttttcctcctgtcccagcttctccgtagactctataaatctaaatggaaaaatggagtaaagcttcctgttaactctcaaacatgtcacagctctccatgtcagcaaaaacaagtgacacagggcttgctcacagatggccccaaagagcgggcagggatgaaccctgtggaatgaggtgtggcctggctcgagaatgactagagtgtggccaaggaagggcagcctcagaaccactgtccgtttacaacgttttccctgcccacatgtctgcctggctttgaaaagattaacaggagtgtttgtttgaaagtcagactcctggtttcctcattagtgaagggatctgctacagacttgggctgtttctataaactttaattacctctgatgaggagtgtatcccctcatcacattcaccccaaaggtacagaggagttcatttttaaaaatgtgttagagcaataaaaggccattagagggagggaggatggggtgtggaagagacgagagagcgagcgagagagagagaaaacacactagctctccctgctggaataataggcttgaaatatgaggaagttgatcaactgccgctgccttccaaaaacagattaatccaccttggtagctttcctttcagagcaagcttttggctctgtcgactttctctatcagcctgaactcaaaaggacacaggccacatgccatctgagcttaagagttattttgtgtgttgatctgagaacttcacattttaaaacaatgaattcatgtttctactgtttgttgctgtctgtggatttgctgatataaagaggagagtcctagcctggtcatttaatgaataattctaaggaacctgaacttctccagggtgcccttctacatctgcagtggtggttctcagctggggtgactttgcttccgtcaccccacccgcaggatatctggcaatgtctggagacattctgagttgtcacaagaggggcgggcgatgctactaacacctaatgggtaaagcagagatgctgccacacatcctacgatgaacgagacagccccctccaccccagcaaatcatgatctggcccaaaatgtcaacagtgtcaacagttgagaaactctgatctacagatatagggaaccctgcctaatacacaaatcctccgtagttcccaagggcggcctgtagcggcacccgacctgacagcgttcagcgacccgcgccagttccccgcgctgccctccatctccgacccccgcatgc

SEQ ID NO: 18 - DNA, 5772 bp, Homo sapiens
  source 1..5772: mol_type = genomic DNA
acagttaaatttgtaatttgggttgtgtgaaaacttctttgggcctcataaacaaccacagaaccacaagttgggtagcctggcagtgtcagaagtctgaacccagcatagtggtcagcaggcaggacgaatcacactgaatgcaaaccacagggtttcgcagcgtggtaaaagaaatcattgagtcccccgccttcagaagagggtgcattttcaggaggaagcgatggcttcagacagcatatttgagtcatttccttcgtacccacagtgcttcatgagagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaaatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcaggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggcccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaactttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaaatgcttctttctactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacatacaccata

SEQ ID NO: 19 - DNA, 5811 bp, Homo sapiens
  source 1..5811: mol_type = genomic DNA
acagttaaatttgtaatttgggttgtgtgaaaacttctttgggcctcataaacaaccacagaaccacaagttgggtagcctggcagtgtcagaagtctgaacccagcatagtggtcagcaggcaggacgaatcacactgaatgcaaaccacagggtttcgcagcgtggtaaaagaaatcattgagtcccccgccttcagaagagggtgcattttcaggaggaagcgatggcttcagacagcatatttgagtcatttccttcgtacccacagtgcttcatgagagaatgcatacttggaatgaatccttctagagacgtccacgatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaaatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcaggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggcccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaactttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaaatgcttctttctactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacatacaccata

SEQ ID NO: 20 - DNA, 5772 bp, Homo sapiens
  source 1..5772: mol_type = genomic DNA
cactttttaatgcactaagcaatcggttgctaggagcccatcctgggtcagaggccgatccgcagaaccagaacgttttcccctcctggactatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcaggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggcccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaactttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaaatgcttctttctactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacatacaccata

SEQ ID NO: 21 - DNA, 5964 bp, Homo sapiens
  source 1..5964: mol_type = genomic DNA
acagttaaatttgtaatttgggttgtgtgaaaacttctttgggcctcataaacaaccacagaaccacaagttgggtagcctggcagtgtcagaagtctgaacccagcatagtggtcagcaggcaggacgaatcacactgaatgcaaaccacagggtttcgcagcgtggtaaaagaaatcattgagtcccccgccttcagaagagggtgcattttcaggaggaagcgatggcttcagacagcatatttgagtcatttccttcgtacccacagtgcttcatgagagatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcaggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggcccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaactttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaaatgcttctttctactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacatacaccata

SEQ ID NO: 22 - DNA, 6078 bp, Homo sapiens
  source 1..6078: mol_type = genomic DNA
gtaatttgggttgtgtgaaaacttctttgggcctcataaacaaccacagaaccacaagttgggtagcctggcagtgtcagaagtctgaacccagcatagtggtcagcaggcaggacgaatcacactgaatgcaaaccacagggtttcgcagcgtggtgagcatcaccaacccacagccaaggcggcgctggctttttttttttttttaatctttaacaatttgaatatttgtttttacaaaggtaaaagaaatcattgagtcccccgccttcagaagagggtgcattttcaggaggaagcgatggcttcagacagcatatttgagtcatttccttcgtacccacagtgcttcatgagagaatgcatacttggaatgaatccttctagagacgtccacgatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaggcgccaggcctggcccggctgggccccgcgggccgccgccttcgcctccgggcgcgcgggcctcctgttcgcgacaagcccgccgggatcccgggccctgggcccggccaccgtcctggggccgagggcgcccgacggccaggatctcgctgtaggtcaggcccgcgcagcctcctgcgcccagaagcccacgccgccgccgtctgctggcgccccggccctcgcggaggtgtccgaggcgacgcacctcgagggtgtccgccggccccagcacccaggggacgcgctggaaagcaaacaggaagattcccggagggaaactgtgaatgcttctgatttagcaatgctgtgaataaaaagaaagattttatacccttgacttaactttttaaccaagttgtttattccaaagagtgtggaattttggttggggtggggggagaggagggatgcaactcgccctgtttggcatctaattcttatttttaatttttccgcaccttatcaattgcaaaatgcgtatttgcatttgggtggtttttatttttatatacgtttatataaatatatataaattgagcttgcttctttcttgctttgaccatggaaagaaatatgattcccttttctttaagttttatttaacttttcttttggacttttgggtagttgtttttttttgttttgttttgtttttttgagaaacagctacagctttgggtcatttttaactactgtattcccacaaggaatccccagatatttatgtatcttgatgttcagacatttatgtgttgataattttttaattatttaaatgtacttatattaagaaaaatatcaagtactacattttcttttgttcttgatagtagccaaagttaaatgtatcacattgaagaaggctagaaaaaaagaatgagtaatgtgatcgcttggttatccagaagtattgtttacattaaactccctttcatgttaatcaaacaagtgagtagctcacgcagcaacgtttttaataggatttttagacactgagggtcactccaaggatcagaagtatggaattttctgccaggctcaacaagggtctcatatctaacttcctccttaaaacagagaaggtcaatctagttccagagggttgaggcaggtgccaataattacatctttggagaggatttgatttctgcccagggatttgctcaccccaaggtcatctgataatttcacagatgctgtgtaacagaacacagccaaagtaaactgtgtaggggagccacatttacataggaaccaaatcaatgaatttaggggttacgattatagcaatttaagggcccaccagaagcaggcctcgaggagtcaatttgcctctgtgtgcctcagtggagacaagtgggaaaacatggtcccacctgtgcgagaccccctgtcctgtgctgctcactcaacaacatctttgtgttgctttcaccaggctgagaccctaccctatggggtatatgggcttttacctgtgcaccagtgtgacaggaaagattcatgtcactactgtccgtggctacaattcaaaggtatccaatgtcgctgtaaattttatggcactatttttattggaggatttggtcagaatgcagttgttgtacaactcataaatactaactgctgattttgacacatgtgtgctccaaatgatctggtggttatttaacgtacctcttaaaattcgttgaaacgatttcaggtcaactctgaagagtatttgaaagcaggacttcagaacagtgtttgatttttattttataaatttaagcattcaaattaggcaaatctttggctgcaggcagcaaaaacagctggacttatttaaaacaacttgtttttgagttttcttatatatatattgattatttgttttacacacatgcagtagcactttggtaagagttaaagagtaaagcagcttatgttgtcaggtcgttcttatctagagaagagctatagcagatctcggacaaactcagaatatattcactttcatttttgacaggattccctccacaactcagtttcatatattattccgtattacatttttgcagctaaattaccataaaatgtcagcaaatgtaaaaatttaatttctgaaaagcaccattagcccatttcccccaaattaaacgtaaatgttttttttcagcacatgttaccatgtctgacctgcaaaaatgctggagaaaaatgaaggaaaaaattatgtttttcagtttaattctgttaactgaagatattccaactcaaaaccagcctcatgctctgattagataatcttttacattgaacctttactctcaaagccatgtgtggagggggcttgtcactattgtaggctcactggattggtcatttagagtttcacagactcttaccagcatatatagtatttaattgtttcaaaaaaaatcaaactgtagttgttttggcgataggtctcacgcaacacatttttgtatgtgtgtgtgtgtgcgtgtgtgtgtgtgtgtgtgaaaaattgcattcattgacttcaggtagattaaggtatctttttattcattgccctcaggaaagttaaggtatcaatgagacccttaagccaatcatgtaataactgcatgtgtctggtccaggagaagtattgaataagccatttctactgcttactcatgtccctatttatgatttcaacatggatacatatttcagttctttctttttctcactatctgaaaatacatttccctccctctcttccccccaatatctccctttttttctctcttcctctatcttccaaaccccactttctccctcctccttttcctgtgttctcttaagcagatagcacatacccccacccagtaccaaatttcagaacacaagaaggtccagttcttcccccttcacataaaggaacatggtttgtcagcctttctcctgtttatgggtttcttccagcagaacagagacattgccaaccatattggatctgcttgctgtccaaaccagcaaactttcctgggcaaatcacaatcagtgagtaaatagacagcctttctgctgccttgggtttctgtgcagataaacagaaatgctctgattagaaaggaaatgaatggttccactcaaatgtcctgcaatttaggattgcagatttctgccttgaaatacctgtttctttgggacattccgtcctgatgatttttatttttgttggtttttatttttggggggaatgacatgtttgggtcttttatacatgaaaatttgtttgacaataatctcacaaaacatattttacatctgaacaaaatgcctttttgtttaccgtagcgtatacatttgttttgggatttttgtgtgtttgttgggaattttgtttttagccaggtcagtattgatgaggctgatcatttggctctttttttccttccagaagagttgcatcaacaaagttaattgtatttatgtatgtaaatagattttaagcttcattataaaatattgttaatgcctataactttttttcaatttttttgtgtgtgtttctaaggactttttcttaggtttgctaaatactgtagggaaaaaaatgcttctttctactttgtttattttagactttaaaatgagctacttcttattcacttttgtaaacagctaatagcatggttccaattttttttaagttcactttttttgttctaggggaaatgaatgtgcaaaaaaagaaaaagaactgttggttatttgtgttattctggatgtataaaaatcaatggaaaaaaataaactttcaaattgaaatgacggtataacacatctactgaaaaagcaacgggaaatgtggtcctatttaagccagcccccacctagggtctatttgtgtggcagttattgggtttggtcacaaaacatcctgaaaattcgtgcgtgggcttctttctccctggtacaaacgtatggaatgcttcttaaaggggaactgtcaagctggtgtcttcagccagatgacatgagagaatatcccagaaccctctctccaaggtgtttctagatagcacaggagagcaggcactgcactgtccacagtccacggtacacagtcgggtgggccgcctcccctctcctgggagcattcgtcgtgcccagcctgagcagggcagctggactgctgctgttcaggagccaccagagccttcctctctttgtaccacagtttcttctgtaaatccagtgttacaatcagtgtgaatggcaaataaacagtttgacaagtacatacaccata

SEQ ID NO: 23 - protein, 521 aa, Homo sapiens
MASNSLFSTVTPCQQNFFWDPSTSRRFSPPSSSLQPGKMSDVSPVVAAQQQQQQQQQQQQQQQQQQQQQQQEAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSTLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSGSYQFPMVPGGDRSPSRMLPPCTTTSNGSTLLNPNLPNQNDGVDADGSHSSSPTVLNSSGRMDESVWRPY

SEQ ID NO: 24 - protein, 499 aa, Homo sapiens
MASNSLFSTVTPCQQNFFWDPSTSRRFSPPSSSLQPGKMSDVSPVVAAQQQQQQQQQQQQQQQQQQQQQQQEAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISGASELGPFSDPRQFPSISSLTESRFSNPRMHYPATFTYTPPVTSGMSLGMSATTHYHTYLPPPYPGSSQSQSGPFQTSSTPYLYYGTSSGSYQFPMVPGGDRSPSRMLPPCTTTSNGSTLLNPNLPNQNDGVDADGSHSSSPTVLNSSGRMDESVWRPY

SEQ ID NO: 25 - protein, 521 aa, Homo sapiens; identical to SEQ ID NO: 23

SEQ ID NO: 26 - protein, 485 aa, Homo sapiens
MRIPVDPSTSRRFSPPSSSLQPGKMSDVSPVVAAQQQQQQQQQQQQQQQQQQQQQQQEAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISGASELGPFSDPRQFPSISSLTESRFSNPRMHYPATFTYTPPVTSGMSLGMSATTHYHTYLPPPYPGSSQSQSGPFQTSSTPYLYYGTSSGSYQFPMVPGGDRSPSRMLPPCTTTSNGSTLLNPNLPNQNDGVDADGSHSSSPTVLNSSGRMDESVWRPY

SEQ ID NO: 27 - protein, 514 aa, Mus musculus
MRIPVDPSTSRRFSPPSSSLQPGKMSDVSPVVAAQQQQQQQQQQQQQQQQQQQQQQQQQQQQQEAAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSSLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSASYQFPMVPGGDRSPSRMVPPCTTTSNGSTLLNPNLPNQNDGVDADGSHSSSPTVLNSSGRMDESVWRPY

SEQ ID NO: 28 - protein, 528 aa, Mus musculus
MASNSLFSAVTPCQQSFFWDPSTSRRFSPPSSSLQPGKMSDVSPVVAAQQQQQQQQQQQQQQQQQQQQQQQQQQQQQEAAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRRHRQKLDDSKPSLFSDRLSDLGRIPHPSMRVGVPPQNPRPSLNSAPSPFNPQGQSQITDPRQAQSSPPWSYDQSYPSYLSQMTSPSIHSTTPLSSTRGTGLPAITDVPRRISDDDTATSDFCLWPSSLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSASYQFPMVPGGDRSPSRMVPPCTTTSNGSTLLNPNLPNQNDGVDADGSHSSSPTVLNSSGRMDESVWRPY

SEQ ID NO: 29 - protein, 528 aa, Mus musculus; identical to SEQ ID NO: 28

SEQ ID NO: 30 - protein, 456 aa, Mus musculus
MRIPVDPSTSRRFSPPSSSLQPGKMSDVSPVVAAQQQQQQQQQQQQQQQQQQQQQQQQQQQQQEAAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRGKSFTLTITVFTNPPQVATYHRAIKVTVDGPREPRNPRQAQSSPPWSYDQSYPSYLSQMTSPSIHSTTPLSSTRGTGLPAITDVPRRISDDDTATSDFCLWPSSLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSASYQFPMVPGGDRSPSRMVPPCTTTSNGSTLLNPNLPNQNDGVDADGSHSSSPTVLNSSGRMDESVWRPY

SEQ ID NO: 31 - protein, 421 aa, Mus musculus
MRIPVDPSTSRRFSPPSSSLQPGKMSDVSPVVAAQQQQQQQQQQQQQQQQQQQQQQQQQQQQQEAAAAAAAAAAAAAAAAAAVPRLRPPHDNRTMVEIIADHPAELVRTDSPNFLCSVLPSHWRCNKTLPVAFKVVALGEVPDGTVVTVMAGNDENYSAELRNASAVMKNQVARFNDLRFVGRSGRDPRQAQSSPPWSYDQSYPSYLSQMTSPSIHSTTPLSSTRGTGLPAITDVPRRISDDDTATSDFCLWPSSLSKKSQAGASELGPFSDPRQFPSISSLTESRFSNPRMHYPATFTYTPPVTSGMSLGMSATTHYHTYLPPPYPGSSQSQSGPFQTSSTPYLYYGTSSASYQFPMVPGGDRSPSRMVPPCTTTSNGSTLLNPNLPNQNDGVDADGSHSSSPTVLNSSGRMDESVWRPY

SEQ ID NO: 32 - DNA, 5487 bp, Homo sapiens
  source 1..5487: mol_type = genomic DNA
gtgtgaatgcttcattcgcctcacaaacaaccacagaaccacaagtgcggtgcaaactttctccaggaggacagcaagaagtctctggtttttaaatggttaatctccgcaggtcactaccagccaccgagaccaacagagtcatttaaggctgcaagcagtatttacaacagagggtacaagttctatctgaaaaaaaaaggagggactatggcatcaaacagcctcttcagcacagtgacaccatgtcagcaaaacttcttttgggatccgagcaccagccggcgcttcagccccccctccagcagcctgcagcccggcaaaatgagcgacgtgagcccggtggtggctgcgcaacagcagcagcaacagcagcagcagcaacagcagcagcagcagcagcaacagcagcagcagcagcaggaggcggcggcggcggctgcggcggcggcggcggctgcggcggcggcagctgcagtgccccggttgcggccgccccacgacaaccgcaccatggtggagatcatcgccgaccacccggccgaactcgtccgcaccgacagccccaacttcctgtgctcggtgctgccctcgcactggcgctgcaacaagaccctgcccgtggccttcaaggtggtagccctcggagaggtaccagatgggactgtggttactgtcatggcgggtaacgatgaaaattattctgctgagctccggaatgcctctgctgttatgaaaaaccaagtagcaaggttcaacgatctgagatttgtgggccggagtggacgaggcaagagtttcaccttgaccataaccgtcttcacaaatcctccccaagtagctacctatcacagagcaattaaagttacagtagatggacctcgggaacccagaaggcacagacagaagcttgatgactctaaacctagtttgttctctgaccgcctcagtgatttagggcgcattcctcatcccagtatgagagtaggtgtcccgcctcagaacccacggccctccctgaactctgcaccaagtccttttaatccacaaggacagagtcagattacagaccccaggcaggcacagtcttccccgccgtggtcctatgaccagtcttacccctcctacctgagccagatgacgtccccgtccatccactctaccaccccgctgtcttccacacggggcactgggcttcctgccatcaccgatgtgcctaggcgcatttcaggtgcttcagaactgggccctttttcagaccccaggcagttcccaagcatttcatccctcactgagagccgcttctccaacccacgaatgcactatccagccacctttacttacaccccgccagtcacctcaggcatgtccctcggtatgtccgccaccactcactaccacacctacctgccaccaccctaccccggctcttcccaaagccagagtggacccttccagaccagcagcactccatatctctactatggcacttcgtcaggatcctatcagtttcccatggtgccggggggagaccggtctccttccagaatgcttccgccatgcaccaccacctcgaatggcagcacgctattaaatccaaatttgcctaaccagaatgatggtgttgacgctgatggaagccacagcagttccccaactgttttgaattctagtggcagaatggatgaatctgtttggcgaccatattgaaattcctcagcagtggcccagtggtatctgggggccacatcccacacgtatcaatatatacatatatagagagagtgcatatatatgtatatcgattagctatctacaaagtgcctattttttagaagatttttcattcactcactcagtcatgatcttgcagccataagagggtagatattgagaagcagaaggctcaagagagacaattgcaatcgagcttcagattgtttactatttaagatgtacttttacaaaggaacaaagaagggaaaaggtatttttgtttttgttgtttggtctgttatcatcaataacctgttcatatgccaattcagagaggtggactccaggttcaggagggagaagagcaaagccgcttcctctctgtgctttgaaacttcacaccctcacggtggcagctgtgtatggaccagtgccctccgcagacagctcacaaaaccagttgaggtgcactaaagggacatgaggtagaatggatgcttccatcacagtaccatcattcagaataactcttccaatttctgctttcagacatgctgcaggtcctcatctgaactgttgggttcgtttttttttttttttttcctgctccaagaaagtgacttcaaaaataactgatcaggatagattattttattttactttttaacactccttctccccttttcccactgaaccaaaaagaaatcccatccctaaaacctgccttctccttttatgcaaaactgaaaatggcaatacattattatagccataatggtatagatagtgattgcgtttggctatgtgttgttttctttttttttaaattatgaatatgtgtaaaatctgaggtaacttgctaacgtgaatggtcatataactttaaagatatatttataattatttaatgacatttggacccttgaaacatttcttagtgtattgatatgttgacttcggtctctaaaagtgctctttattaaataacaaatttcttcagtggtctagagccatatctgaaatattgctaagcaatttcagttcatccaggcacaatgtgattttaaaaaatacttccatctccaaatattttagatatagattgtttttgtgatgtatgaaggaaatgttatgtttagttctttcagatctttgaatgcctctaacacagctttgccttctaaagcggtaattagggatttaaaaaacaacctttagccctttatcagcatgaaatgctggagtgatgtggttttctaatttctttggggtaattatgactcttgtcatattaaaaagacaagcacaagtaaatcattgaactacagaaaaatgttctgtggtttcatagttaagcaaaactctaaatcgccaggcttcatagcaaagacatagtcagctaaaagccgcacatgtggatagagggttcaattatgagacacctagtacaggagagcaaaattgcaccagagattcttaaccaaccagccttaccaaacaacacaacaggggaaccccaatctgccttacccaaggccccactggcagctttccacagaatttgcatttagaggagcagaatgacatcactgtcctttgggagtaggtcctctgaaaaggcagcaggttccagcaggtagctgagctgagaggacatatggcccacggggacctacagacagcctttgacatttgtatttcttacaatggagggccaaggagggcaaggggctgtggagtttggtgtctactagtgtgtatgaatttgagctagagtccttctgtggcatgcactttgaccactcctggcagtcacatggcagatttccaagtgcaaatccttaatccaaacaaggatcatctaatgacaccaccaggccaatccctgctctcctccccgaaaagtcagggtcccttcattggaatcctccacccacccaagcagaatttagcagagatttgccttcaaaccctaacggcccccttgttctctggtccttctcaaacccacctttgtaggccacccagcattgcaggacagcgtgtggggcagctggacctgtgcttcctgcctgggagtctcccttggaattcatcctgactccttctaataaaaatggatgggaaagcaaaacactttgccttctaaaggccgtataccaagtatgcttagataaataagccacttttctattacttaagtaagaaggaagtagtaattgatactatttattgtttgtgtgtggtagcttgaagcacaccactgtccatttatttgtaagtgtaaaatatgtgtgtttgtttcagcagcacttaaaaaagccagtgtctggttacacatttcaattttaattaattgacataaaaatgctaccgccagtgccagctgcatcctatttaattaaaaaggtactatatttgtacattattttttaatgttaaaagggcttttttaagtttacagtacacataccgagtgactttagggatgcttttgtgttgaaatgttactatagtggctgcaggcagcaacccagaaacactttagaagctttttttccttgggaaaaattcaagcacttcttccctccaccctcactccaaccaccccaatgggggtaattcacatttcttagaacaaattctgcccttttttggtctagggattaaaattttgtttttctttctttctttttttttttttttcactgaacccttaatttgcactgggtcatgtgtttgatttgtgatttcaagaccaaagcaaagtcttactactactgtggaaccatgtactagttcctgggaattaaaatagcgtggttctctttgtagcacaaacattgctggaatttgcagtcttttcaatgcagccacatttttatccatttcagttgtctcacaaattttaacccatatcagagttccagaacaggtaccacagctttggttttagattagtggaataacattcagcccagaactgagaaactcaacagattaactatcgtttgctctttagacggtctcactgcctctcacttgccagagccctttcaaaatgagcagagaagtccacaccattagggaccatctgtgataaattcagaagggaggagatgtgtgtacagctttaaggattccctcaattccgaggaaagggactggcccagaatccaggttaatacatggaaacacgaagcattagcaaaagtaataattatacctatggtatttgaaagaacaataataaaagacacttcttccaaaccttgaatttgttgtttttagaaaacgaatgcatttaaaaatattttctatgtgagaattttttagatgtgtgtttacttcatgtttacaaataactgtttgctttttaatgcagtactttgaaatatatcagccaaaaccataacttacaataatttcttaggtattctgaataaaattccatttcttttggatatgctttaccattcttaggtttctgtggaacaaaaatatttgtagcattttgtgtaaatacaagctttcatttttattttttccaattgctattgcccaagaattgctttccatgcacatattgtaaaaattccgctttgtgccacaggtcatgattgtggatgagtttactcttaacttcaaagggactatttgtattgtatgttgcaactgtaaattgaattatttggcatttttctcatgattgtaatattaatttgaagtttgaatttaattttcaataaaatggcttttttggttttgtta

SEQ ID NO: 33 - DNA, 5553 bp, Homo sapiens
  source 1..5553: mol_type = genomic DNA
gtgtgaatgcttcattcgcctcacaaacaaccacagaaccacaagtgcggtgcaaactttctccaggaggacagcaagaagtctctggtttttaaatggttaatctccgcaggtcactaccagccaccgagaccaacagagtcatttaaggctgcaagcagtatttacaacagagggtacaagttctatctgaaaaaaaaaggagggactatggcatcaaacagcctcttcagcacagtgacaccatgtcagcaaaacttcttttgggatccgagcaccagccggcgcttcagccccccctccagcagcctgcagcccggcaaaatgagcgacgtgagcccggtggtggctgcgcaacagcagcagcaacagcagcagcagcaacagcagcagcagcagcagcaacagcagcagcagcagcaggaggcggcggcggcggctgcggcggcggcggcggctgcggcggcggcagctgcagtgccccggttgcggccgccccacgacaaccgcaccatggtggagatcatcgccgaccacccggccgaactcgtccgcaccgacagccccaacttcctgtgctcggtgctgccctcgcactggcgctgcaacaagaccctgcccgtggccttcaaggtggtagccctcggagaggtaccagatgggactgtggttactgtcatggcgggtaacgatgaaaattattctgctgagctccggaatgcctctgctgttatgaaaaaccaagtagcaaggttcaacgatctgagatttgtgggccggagtggacgaggcaagagtttcaccttgaccataaccgtcttcacaaatcctccccaagtagctacctatcacagagcaattaaagttacagtagatggacctcgggaacccagaaggcacagacagaagcttgatgactctaaacctagtttgttctctgaccgcctcagtgatttagggcgcattcctcatcccagtatgagagtaggtgtcccgcctcagaacccacggccctccctgaactctgcaccaagtccttttaatccacaaggacagagtcagattacagaccccaggcaggcacagtcttccccgccgtggtcctatgaccagtcttacccctcctacctgagccagatgacgtccccgtccatccactctaccaccccgctgtcttccacacggggcactgggcttcctgccatcaccgatgtgcctaggcgcatttcagatgatgacactgccacctctgacttctgcctctggccttccactctcagtaagaagagccaggcaggtgcttcagaactgggccctttttcagaccccaggcagttcccaagcatttcatccctcactgagagccgcttctccaacccacgaatgcactatccagccacctttacttacaccccgccagtcacctcaggcatgtccctcggtatgtccgccaccactcactaccacacctacctgccaccaccctaccccggctcttcccaaagccagagtggacccttccagaccagcagcactccatatctctactatggcacttcgtcaggatcctatcagtttcccatggtgccggggggagaccggtctccttccagaatgcttccgccatgcaccaccacctcgaatggcagcacgctattaaatccaaatttgcctaaccagaatgatggtgttgacgctgatggaagccacagcagttccccaactgttttgaattctagtggcagaatggatgaatctgtttggcgaccatattgaaattcctcagcagtggcccagtggtatctgggggccacatcccacacgtatcaatatatacatatatagagagagtgcatatatatgtatatcgattagctatctacaaagtgcctattttttagaagatttttcattcactcactcagtcatgatcttgcagccataagagggtagatattgagaagcagaaggctcaagagagacaattgcaatcgagcttcagattgtttactatttaagatgtacttttacaaaggaacaaagaagggaaaaggtatttttgtttttgttgtttggtctgttatcatcaataacctgttcatatgccaattcagagaggtggactccaggttcaggagggagaagagcaaagccgcttcctctctgtgctttgaaacttcacaccctcacggtggcagctgtgtatggaccagtgccctccgcagacagctcacaaaaccagttgaggtgcactaaagggacatgaggtagaatggatgcttccatcacagtaccatcattcagaataactcttccaatttctgctttcagacatgctgcaggtcctcatctgaactgttgggttcgtttttttttttttttttcctgctccaagaaagtgacttcaaaaataactgatcaggatagattattttattttactttttaacactccttctccccttttcccactgaaccaaaaagaaatcccatccctaaaacctgccttctccttttatgcaaaactgaaaatggcaatacattattatagccataatggtatagatagtgattgcgtttggctatgtgttgttttctttttttttaaattatgaatatgtgtaaaatctgaggtaacttgctaacgtgaatggtcatataactttaaagatatatttataattatttaatgacatttggacccttgaaacatttcttagtgtattgatatgttgacttcggtctctaaaagtgctctttattaaataacaaatttcttcagtggtctagagccatatctgaaatattgctaagcaatttcagttcatccaggcacaatgtgattttaaaaaatacttccatctccaaatattttagatatagattgtttttgtgatgtatgaaggaaatgttatgtttagttctttcagatctttgaatgcctctaacacagctttgccttctaaagcggtaattagggatttaaaaaacaacctttagccctttatcagcatgaaatgctggagtgatgtggttttctaatttctttggggtaattatgactcttgtcatattaaaaagacaagcacaagtaaatcattgaactacagaaaaatgttctgtggtttcatagttaagcaaaactctaaatcgccaggcttcatagcaaagacatagtcagctaaaagccgcacatgtggatagagggttcaattatgagacacctagtacaggagagcaaaattgcaccagagattcttaaccaaccagccttaccaaacaacacaacaggggaaccccaatctgccttacccaaggccccactggcagctttccacagaatttgcatttagaggagcagaatgacatcactgtcctttgggagtaggtcctctgaaaaggcagcaggttccagcaggtagctgagctgagaggacatatggcccacggggacctacagacagcctttgacatttgtatttcttacaatggagggccaaggagggcaaggggctgtggagtttggtgtctactagtgtgtatgaatttgagctagagtccttctgtggcatgcactttgaccactcctggcagtcacatggcagatttccaagtgcaaatccttaatccaaacaaggatcatctaatgacaccaccaggccaatccctgctctcctccccgaaaagtcagggtcccttcattggaatcctccacccacccaagcagaatttagcagagatttgccttcaaaccctaacggcccccttgttctctggtccttctcaaacccacctttgtaggccacccagcattgcaggacagcgtgtggggcagctggacctgtgcttcctgcctgggagtctcccttggaattcatcctgactccttctaataaaaatggatgggaaagcaaaacactttgccttctaaaggccgtataccaagtatgcttagataaataagccacttttctattacttaagtaagaaggaagtagtaattgatactatttattgtttgtgtgtggtagcttgaagcacaccactgtccatttatttgtaagtgtaaaatatgtgtgtttgtttcagcagcacttaaaaaagccagtgtctggttacacatttcaattttaattaattgacataaaaatgctaccgccagtgccagctgcatcctatttaattaaaaaggtactatatttgtacattattttttaatgttaaaagggcttttttaagtttacagtacacataccgagtgactttagggatgcttttgtgttgaaatgttactatagtggctgcaggcagcaacccagaaacactttagaagctttttttccttgggaaaaattcaagcacttcttccctccaccctcactccaaccaccccaatgggggtaattcacatttcttagaacaaattctgcccttttttggtctagggattaaaattttgtttttctttctttctttttttttttttttcactgaacccttaatttgcactgggtcatgtgtttgatttgtgatttcaagaccaaagcaaagtcttactactactgtggaaccatgtactagttcctgggaattaaaatagcgtggttctctttgtagcacaaacattgctggaatttgcagtcttttcaatgcagccacatttttatccatttcagttgtctcacaaattttaacccatatcagagttccagaacaggtaccacagctttggttttagattagtggaataacattcagcccagaactgagaaactcaacagattaactatcgtttgctctttagacggtctcactgcctctcacttgccagagccctttcaaaatgagcagagaagtccacaccattagggaccatctgtgataaattcagaagggaggagatgtgtgtacagctttaaggattccctcaattccgaggaaagggactggcccagaatccaggttaatacatggaaacacgaagcattagcaaaagtaataattatacctatggtatttgaaagaacaataataaaagacacttcttccaaaccttgaatttgttgtttttagaaaacgaatgcatttaaaaatattttctatgtgagaattttttagatgtgtgtttacttcatgtttacaaataactgtttgctttttaatgcagtactttgaaatatatcagccaaaaccataacttacaataatttcttaggtattctgaataaaattccatttcttttggatatgctttaccattcttaggtttctgtggaacaaaaatatttgtagcattttgtgtaaatacaagctttcatttttattttttccaattgctattgcccaagaattgctttccatgcacatattgtaaaaattccgctttgtgccacaggtcatgattgtggatgagtttactcttaacttcaaagggactatttgtattgtatgttgcaactgtaaattgaattatttggcatttttctcatgattgtaatattaatttgaagtttgaatttaattttcaataaaatggcttttttggttttgtta

SEQ ID NO: 34 - DNA, 5235 bp, Homo sapiens
  source 1..5235: mol_type = genomic DNA
atgcgtattcccgtagatccgagcaccagccggcgcttcagccccccctccagcagcctgcagcccggcaaaatgagcgacgtgagcccggtggtggctgcgcaacagcagcagcaacagcagcagcagcaacagcagcagcagcagcagcaacagcagcagcagcagcaggaggcggcggcggcggctgcggcggcggcggcggctgcggcggcggcagctgcagtgccccggttgcggccgccccacgacaaccgcaccatggtggagatcatcgccgaccacccggccgaactcgtccgcaccgacagccccaacttcctgtgctcggtgctgccctcgcactggcgctgcaacaagaccctgcccgtggccttcaaggtggtagccctcggagaggtaccagatgggactgtggttactgtcatggcgggtaacgatgaaaattattctgctgagctccggaatgcctctgctgttatgaaaaaccaagtagcaaggttcaacgatctgagatttgtgggccggagtggacgaggcaagagtttcaccttgaccataaccgtcttcacaaatcctccccaagtagctacctatcacagagcaattaaagttacagtagatggacctcgggaacccagaaggcacagacagaagcttgatgactctaaacctagtttgttctctgaccgcctcagtgatttagggcgcattcctcatcccagtatgagagtaggtgtcccgcctcagaacccacggccctccctgaactctgcaccaagtccttttaatccacaaggacagagtcagattacagaccccaggcaggcacagtcttccccgccgtggtcctatgaccagtcttacccctcctacctgagccagatgacgtccccgtccatccactctaccaccccgctgtcttccacacggggcactgggcttcctgccatcaccgatgtgcctaggcgcatttcaggtgcttcagaactgggccctttttcagaccccaggcagttcccaagcatttcatccctcactgagagccgcttctccaacccacgaatgcactatccagccacctttacttacaccccgccagtcacctcaggcatgtccctcggtatgtccgccaccactcactaccacacctacctgccaccaccctaccccggctcttcccaaagccagagtggacccttccagaccagcagcactccatatctctactatggcacttcgtcaggatcctatcagtttcccatggtgccggggggagaccggtctccttccagaatgcttccgccatgcaccaccacctcgaatggcagcacgctattaaatccaaatttgcctaaccagaatgatggtgttgacgctgatggaagccacagcagttccccaactgttttgaattctagtggcagaatggatgaatctgtttggcgaccatattgaaattcctcagcagtggcccagtggtatctgggggccacatcccacacgtatcaatatatacatatatagagagagtgcatatatatgtatatcgattagctatctacaaagtgcctattttttagaagatttttcattcactcactcagtcatgatcttgcagccataagagggtagatattgagaagcagaaggctcaagagagacaattgcaatcgagcttcagattgtttactatttaagatgtacttttacaaaggaacaaagaagggaaaaggtatttttgtttttgttgtttggtctgttatcatcaataacctgttcatatgccaattcagagaggtggactccaggttcaggagggagaagagcaaagccgcttcctctctgtgctttgaaacttcacaccctcacggtggcagctgtgtatggaccagtgccctccgcagacagctcacaaaaccagttgaggtgcactaaagggacatgaggtagaatggatgcttccatcacagtaccatcattcagaataactcttccaatttctgctttcagacatgctgcaggtcctcatctgaactgttgggttcgtttttttttttttttttcctgctccaagaaagtgacttcaaaaataactgatcaggatagattattttattttactttttaacactccttctccccttttcccactgaaccaaaaagaaatcccatccctaaaacctgccttctccttttatgcaaaactgaaaatggcaatacattattatagccataatggtatagatagtgattgcgtttggctatgtgttgttttctttttttttaaattatgaatatgtgtaaaatctgaggtaacttgctaacgtgaatggtcatataactttaaagatatatttataattatttaatgacatttggacccttgaaacatttcttagtgtattgatatgttgacttcggtctctaaaagtgctctttattaaataacaaatttcttcagtggtctagagccatatctgaaatattgctaagcaatttcagttcatccaggcacaatgtgattttaaaaaatacttccatctccaaatattttagatatagattgtttttgtgatgtatgaaggaaatgttatgtttagttctttcagatctttgaatgcctctaacacagctttgccttctaaagcggtaattagggatttaaaaaacaacctttagccctttatcagcatgaaatgctggagtgatgtggttttctaatttctttggggtaattatgactcttgtcatattaaaaagacaagcacaagtaaatcattgaactacagaaaaatgttctgtggtttcatagttaagcaaaactctaaatcgccaggcttcatagcaaagacatagtcagctaaaagccgcacatgtggatagagggttcaattatgagacacctagtacaggagagcaaaattgcaccagagattcttaaccaaccagccttaccaaacaacacaacaggggaaccccaatctgccttacccaaggccccactggcagctttccacagaatttgcatttagaggagcagaatgacatcactgtcctttgggagtaggtcctctgaaaaggcagcaggttccagcaggtagctgagctgagaggacatatggcccacggggacctacagacagcctttgacatttgtatttcttacaatggagggccaaggagggcaaggggctgtggagtttggtgtctactagtgtgtatgaatttgagctagagtccttctgtggcatgcactttgaccactcctggcagtcacatggcagatttccaagtgcaaatccttaatccaaacaaggatcatctaatgacaccaccaggccaatccctgctctcctccccgaaaagtcagggtcccttcattggaatcctccacccacccaagcagaatttagcagagatttgccttcaaaccctaacggcccccttgttctctggtccttctcaaacccacctttgtaggccacccagcattgcaggacagcgtgtggggcagctggacctgtgcttcctgcctgggagtctcccttggaattcatcctgactccttctaataaaaatggatgggaaagcaaaacactttgccttctaaaggccgtataccaagtatgcttagataaataagccacttttctattacttaagtaagaaggaagtagtaattgatactatttattgtttgtgtgtggtagcttgaagcacaccactgtccatttatttgtaagtgtaaaatatgtgtgtttgtttcagcagcacttaaaaaagccagtgtctggttacacatttcaattttaattaattgacataaaaatgctaccgccagtgccagctgcatcctatttaattaaaaaggtactatatttgtacattattttttaatgttaaaagggcttttttaagtttacagtacacataccgagtgactttagggatgcttttgtgttgaaatgttactatagtggctgcaggcagcaacccagaaacactttagaagctttttttccttgggaaaaattcaagcacttcttccctccaccctcactccaaccaccccaatgggggtaattcacatttcttagaacaaattctgcccttttttggtctagggattaaaattttgtttttctttctttctttttttttttttttcactgaacccttaatttgcactgggtcatgtgtttgatttgtgatttcaagaccaaagcaaagtcttactactactgtggaaccatgtactagttcctgggaattaaaatagcgtggttctctttgtagcacaaacattgctggaatttgcagtcttttcaatgcagccacatttttatccatttcagttgtctcacaaattttaacccatatcagagttccagaacaggtaccacagctttggttttagattagtggaataacattcagcccagaactgagaaactcaacagattaactatcgtttgctctttagacggtctcactgcctctcacttgccagagccctttcaaaatgagcagagaagtccacaccattagggaccatctgtgataaattcagaagggaggagatgtgtgtacagctttaaggattccctcaattccgaggaaagggactggcccagaatccaggttaatacatggaaacacgaagcattagcaaaagtaataattatacctatggtatttgaaagaacaataataaaagacacttcttccaaaccttgaatttgttgtttttagaaaacgaatgcatttaaaaatattttctatgtgagaattttttagatgtgtgtttacttcatgtttacaaataactgtttgctttttaatgcagtactttgaaatatatcagccaaaaccataacttacaataatttcttaggtattctgaataaaattccatttcttttggatatgctttaccattcttaggtttctgtggaacaaaaatatttgtagcattttgtgtaaatacaagctttcatttttattttttccaattgctattgcccaagaattgctttccatgcacatattgtaaaaattccgctttgtgccacaggtcatgattgtggatgagtttactcttaacttcaaagggactatttgtattgtatgttgcaactgtaaattgaattatttggcatttttctcatgattgtaatattaatttgaagtttgaatttaattttcaataaaatggcttttttggttttgtta

SEQ ID NO: 35 - DNA, 6475 bp, Mus musculus
  source 1..6475: mol_type = genomic DNA
ctgaagttaacaacgaaaaattaacgccagtcggagcagcctgaggctctcccgcttctcagctttagcgtcgtcagaccgagaagtggttcccggtcctgagggtgaagtggggagccgaggacgcaggcggcgatgtcctaggcggggacctcctccctacagcttcgggcgccgagcgagcgcagcggcgcttctagcggccggcgggcggcggcagcggctgcgatccgcaggctccagatctgtcgccccgagatccgctctccccccgcccccacttacctcccggcaccttgaaacgcgaggggggcccggggcactttgcaaagagcaggagagacgaggctgcgagctagacggcggcgaaggagagggcgagaggagaagccggggaaaggaaggactcggcggccggaggactcggagcgcgctgccggcgcggggagcgcgcagcggcctcggaggaggaggcggaggaggcggcgcgggcaagcgacggcgccgcgagctgggcagccgcgctctgcttggcggtggcggacagcgaggagccacacgcaccgccgagatggactgctgaacctgcggggctccactaccgtactggaacccggagcggggcgccagcgcacccaggacacggtgccccaaggggcccctactgcaagctgttaacttcaagtcccttgcggcggctgggccaaacacgcccccgcgtcccgctcgttgcagccaccgccagggactcccaagcctcccggtggagatccgcgcctctcgggtgtccccacccgctaccccgactctgtccggtctccagtcgccggcccccagcagactccgcccggcacacggcttcctcgaacttgatttctcacctcctctgtcccgtcacctccatcctctttccgcccccgtctcgcctccaccccctcgatttcctcctcctcgccccccatttccaccctcctccccctcccccggccacttcgctaacttgtggctgttgtgatgcgtattcctgtagatccgagcaccagccggcgcttcagccccccctccagcagcctgcagcccggcaagatgagcgacgtgagcccggtggtggctgcgcagcagcagcaacagcagcagcagcagcagcaacagcagcagcaacaacagcaacagcaacaacagcagcagcagcagcagcagcaggaggcggccgcagcagcagcggcggcagcggcggcggcagcagcggcggcggccgcagtgccccgattgaggccgccgcacgacaaccgcaccatggtggagatcatcgcggaccacccggccgaactggtccgcaccgacagtcccaacttcctgtgctccgtgctgccctcgcactggcggtgcaacaagaccctgcccgtggccttcaaggttgtagccctcggagaggtaccagatgggactgtggttaccgtcatggccgggaatgatgagaactactccgccgagctccgaaatgcctccgctgttatgaaaaaccaagtagccaggttcaacgatctgagatttgtgggccggagcggacgaggcaagagtttcaccttgaccataacagtcttcacaaatcctccccaagtggccacttaccacagagctattaaagtgacagtggacggtccccgggaaccaagaaggcacagacagaagcttgatgactctaaacctagtttgttctctgatcgcctcagtgatttagggcgcattcctcatcccagtatgagagtaggtgtcccgcctcagaacccacggccctccctgaactctgcaccaagtccttttaatccacaaggacagagtcagattacagatcccaggcaggcacagtcttccccaccgtggtcctatgaccagtcttacccctcctatctgagccagatgacatccccatccatccactccaccacgccgctgtcttccacacggggcaccgggctacctgccatcactgacgtgcccaggcgtatttcagatgatgacactgccacctctgacttctgcctctggccttcctctctcagtaagaagagccaggcaggtgcttcagaactgggccctttttcagaccccaggcagttcccaagcatttcatccctcactgagagccgcttctccaacccacgaatgcactacccagccacctttacctacaccccgccagtcacgtcaggcatgtccctcggcatgtccgccaccactcactaccacacgtacctgccaccaccctaccccggctcttcccaaagccagagtggacccttccagaccagcagcactccatatctctactatggtacttcgtcagcatcctatcagttcccaatggtacccgggggagaccggtctccttccaggatggtcccaccatgcaccaccacctcgaatggcagcacgctattaaatccaaatttgcctaaccagaatgatggtgttgacgctgacggaagccacagcagttccccaactgttttgaattctagcggcagaatggatgagtctgtttggcggccatattgaaattcgtcaaccatggcccagtggcatgggggccacatcccgcatgtgttaatatatacatatataaagagagtgcctatatatgtatattgattagctaactagaagatttctcattcaatccctagtcatgatcttgcaaccctaagagggtgggggcagtcataactgggtttcatattgtttactatttaagatgtcccctttaccaaggaacaaaccgtcaaaggtgttgtctggtctgttttcataagtgacctgttcccacgccggttcagagaggtggactctgggtctgggaggaaggagagacacttcctctctgtgctttgaaaccacagcctctgctgtgtggcagccggtacactctgcagacccgcttacagagtcagatgtggtgcactcagaaagggacaagaggcagagtggctgcttctgtccgctgccgtccactctgccgtccacctgttccaaagttttccttcagacttgctgcaggtactcatttgaacttttgagttcactttttttttttcctattctaagaaagtgacttcaaaaatactgatcaggacagataattttattttaccttttatattttctcacttcccccatttaaccaaaaagaaatcccgttccccctcccccgttccttctgcttctccctttatgcaaactgaaaatggcaatgccttattattatagccataatggtatagtgtttgagttggctgtgtgttatgtgtttttttctttttttttcttttttaaattatgaatatgtgtaaaatctgaagtaacttgctaacgtgaatggtcatataactttaaagatatatttataattatttaatgacatttggacatttggaacatttcttagtgtaatgatatgttgacttcggtctctaaaagtgtgcttcttcttcaataccaagtttcttcagtgggctagagccatatcggaaatattgctaagcaatctcaattccttcaggcataatgtgatttttttttttttttgaagataactcccatctccaaatagtttagatgtagtttgttttcacgatgtatgaaggagatgctctgtttctttctttcaggcatttgattgcctctgacacagctttgccttttaaagcaataattagggattaaaataacaaaaacaaaacaaaagccacctatagccctttaacacttaacgtggcccctttactagcatgaaatgctggagacatgtggtttcctaatttctccattttgggggtggtgggaggggggagggtggccattatgactcttatcatattaaaaagccaagcacaagtgattggttgaactgcagaaaagtgttctgtggtctctgagttgagcaaaactctaaattgcaggcttcgtggttgagggcctagtcagctgaaagccacgcgtgtggtaaaggctcaggcatggcttggagaacctaggaacacattaggagcctgcacctaccagcctcaccatacagccattcaggggaacccaaaaagtgccttacccaaggagggcccccagcagctttccaggaagtcgaatgaagtcgctgtcctcggggaactggtcagctgaagtagcaaccggtagctgatgtcagtagacaacagaacctgtggggacctccaggaaacctttgacattggaggctttcattaggcagggccaacaagagcagggaaggccatgtacccattggtatctgccattgtgtgtgagtttgagatccagccctccttggaggatgtactgtgatcattcctggtaccttagggccaatccctaagtgtggcttcctaatccaggccagggatcattcagtgataccaccaggccaatcccagcattcctcctgcacaaagtgtttgtgtgtggggggatagattggggtggggccaccttcatttgaatcctgagtcattctaagagttctgcaagcttttgccttcagcaccctataccccctcgctctctgttccttctcaggttgacctttgtcccaatgcgggacagtccagaggcagatggggacctatgtgtgcctccaacctgcgttttcctcagaattcatcctgactccctctgacacagattgaggggggggggaagaaacccaacccgcaccaaagcaaaacactttgccttctaaaggctgtgcaccaagtagacgcagatggtcagcccacctttgtgtttccttaagatggaaattgtaactgatgctatttattgtttgtgtgtggtagcttgaagcacaccacggtccatgtgtttgtctgatacctatttcagcagcatttacaaaagccagtgtctggttacacttttcagttttcattaatcaacatgaaaatgttaccattggtgccagctgcaccctatttaatttttttaagggcactatatttgtacatttcgtttttaatgttaaagggcttcttaaagtttacagtacagttatcaagggaatagaggggatgcattagtgcctaaatgttattctagtggctgcaggcagcaacccagaagcagttttgaaaacaggttgtttccctctgtcctcccttatttgggaaaattcaagtgctttcttcacctttcaggcacctcacggtgactcccgttacttagagcagtctgtcgtcgtcttcttcttcttcctcttcctcttcctcttcctcttcctcttcctcttcctcttcctcttcctcttcctcttcttcttcttcttcttcttcttcttcttcttcttcttcctcctagggtttaaaatcccccttcctcttcccttatctcttaaactcttcatttgcactgggtcacacgtatgatttgtggttttaagaccaaagcaatgtcttattactcttctggagccgtttatgtgtactaaccaacccttccctccacttccctgggtttagatgcacacggttctcaaaggagcacaaacatggccagattcacagtgggacccacacagccatgttaaaaaaaaaaacaaaaaaaaacctcttcacttgtctgagaattttaacctgggccccaattgctaacgggcaccatggcttgggtttcaggttagggaacgttgcccagtgagtaacagaaagacttaactgatttaattagtttgccctcatccttcactccaagaccctaagaaaccgatcagagaaacatctccacaccattagaggttgagaagggaggagccatgtgggggttccctccgctcggaggaaaggcactgactgacctagttagagtggtagcagaagcacccatggtatctgagagagcaataaacaataaaagatgatcctcccaagcttagaatttgttgttcttaaagaagacgatgcatctaaaacaaagaatttttttttctatgtgggaactttcttcgttgtctgtttacttcatgtttacaaataattgtttgctttttaatgcagtactttaaaaaatatatcagccaaaaccataacttacagtaatttttttaggtattctgaataaaattccatgtcttttgatatgtctactgttcttaggtttctgtggaacaacaacaacaacaacaacaaaattgtagcattttgtgtaaatacagctttcgttcttattttttatttttctgattgctattgcccaagatttgctttctatgcacgtattgtacaaattgtgctttgtgccacaggtcatgatcgtggatgagtttactctgaacttcaaagggactatttgtattgtatgttgcaactgtaaattgaattatttggcatttccccctctcatgattgtaatattaatttgaagtttgaatttaattttcaataaaaaggctttttttttccttttggttttg

SEQ ID NO: 36 - DNA, 5740 bp, Mus musculus
  source 1..5740: mol_type = genomic DNA
atgcttcattcgcctcacaaacaaccacagaaccacaagtgcggtgcaaactttctccaggaagactgcaagaaggctctggcgtttaaatggttaatctctgcaggtcactaccagccaccgagaccaaccgagtcagtgagtgctctaaccacagtccatgcaggaatatttaaggctgcaagcagtatttacaacagagggcacaagttctatctggaaaaaaaaggagggactatggcgtcaaacagcctcttcagcgcagtgacaccgtgtcagcaaagcttcttttgggatccgagcaccagccggcgcttcagccccccctccagcagcctgcagcccggcaagatgagcgacgtgagcccggtggtggctgcgcagcagcagcaacagcagcagcagcagcagcaacagcagcagcaacaacagcaacagcaacaacagcagcagcagcagcagcagcaggaggcggccgcagcagcagcggcggcagcggcggcggcagcagcggcggcggccgcagtgccccgattgaggccgccgcacgacaaccgcaccatggtggagatcatcgcggaccacccggccgaactggtccgcaccgacagtcccaacttcctgtgctccgtgctgccctcgcactggcggtgcaacaagaccctgcccgtggccttcaaggttgtagccctcggagaggtaccagatgggactgtggttaccgtcatggccgggaatgatgagaactactccgccgagctccgaaatgcctccgctgttatgaaaaaccaagtagccaggttcaacgatctgagatttgtgggccggagcggacgaggcaagagtttcaccttgaccataacagtcttcacaaatcctccccaagtggccacttaccacagagctattaaagtgacagtggacggtccccgggaaccaagaaggcacagacagaagcttgatgactctaaacctagtttgttctctgatcgcctcagtgatttagggcgcattcctcatcccagtatgagagtaggtgtcccgcctcagaacccacggccctccctgaactctgcaccaagtccttttaatccacaaggacagagtcagattacagatcccaggcaggcacagtcttccccaccgtggtcctatgaccagtcttacccctcctatctgagccagatgacatccccatccatccactccaccacgccgctgtcttccacacggggcaccgggctacctgccatcactgacgtgcccaggcgtatttcagatgatgacactgccacctctgacttctgcctctggccttcctctctcagtaagaagagccaggcaggtgcttcagaactgggccctttttcagaccccaggcagttcccaagcatttcatccctcactgagagccgcttctccaacccacgaatgcactacccagccacctttacctacaccccgccagtcacgtcaggcatgtccctcggcatgtccgccaccactcactaccacacgtacctgccaccaccctaccccggctcttcccaaagccagagtggacccttccagaccagcagcactccatatctctactatggtacttcgtcagcatcctatcagttcccaatggtacccgggggagaccggtctccttccaggatggtcccaccatgcaccaccacctcgaatggcagcacgctattaaatccaaatttgcctaaccagaatgatggtgttgacgctgacggaagccacagcagttccccaactgttttgaattctagcggcagaatggatgagtctgtttggcggccatattgaaattcgtcaaccatggcccagtggcatgggggccacatcccgcatgtgttaatatatacatatataaagagagtgcctatatatgtatattgattagctaactagaagatttctcattcaatccctagtcatgatcttgcaaccctaagagggtgggggcagtcataactgggtttcatattgtttactatttaagatgtcccctttaccaaggaacaaaccgtcaaaggtgttgtctggtctgttttcataagtgacctgttcccacgccggttcagagaggtggactctgggtctgggaggaaggagagacacttcctctctgtgctttgaaaccacagcctctgctgtgtggcagccggtacactctgcagacccgcttacagagtcagatgtggtgcactcagaaagggacaagaggcagagtggctgcttctgtccgctgccgtccactctgccgtccacctgttccaaagttttccttcagacttgctgcaggtactcatttgaacttttgagttcactttttttttttcctattctaagaaagtgacttcaaaaatactgatcaggacagataattttattttaccttttatattttctcacttcccccatttaaccaaaaagaaatcccgttccccctcccccgttccttctgcttctccctttatgcaaactgaaaatggcaatgccttattattatagccataatggtatagtgtttgagttggctgtgtgttatgtgtttttttctttttttttcttttttaaattatgaatatgtgtaaaatctgaagtaacttgctaacgtgaatggtcatataactttaaagatatatttataattatttaatgacatttggacatttggaacatttcttagtgtaatgatatgttgacttcggtctctaaaagtgtgcttcttcttcaataccaagtttcttcagtgggctagagccatatcggaaatattgctaagcaatctcaattccttcaggcataatgtgatttttttttttttttgaagataactcccatctccaaatagtttagatgtagtttgttttcacgatgtatgaaggagatgctctgtttctttctttcaggcatttgattgcctctgacacagctttgccttttaaagcaataattagggattaaaataacaaaaacaaaacaaaagccacctatagccctttaacacttaacgtggcccctttactagcatgaaatgctggagacatgtggtttcctaatttctccattttgggggtggtgggaggggggagggtggccattatgactcttatcatattaaaaagccaagcacaagtgattggttgaactgcagaaaagtgttctgtggtctctgagttgagcaaaactctaaattgcaggcttcgtggttgagggcctagtcagctgaaagccacgcgtgtggtaaaggctcaggcatggcttggagaacctaggaacacattaggagcctgcacctaccagcctcaccatacagccattcaggggaacccaaaaagtgccttacccaaggagggcccccagcagctttccaggaagtcgaatgaagtcgctgtcctcggggaactggtcagctgaagtagcaaccggtagctgatgtcagtagacaacagaacctgtggggacctccaggaaacctttgacattggaggctttcattaggcagggccaacaagagcagggaaggccatgtacccattggtatctgccattgtgtgtgagtttgagatccagccctccttggaggatgtactgtgatcattcctggtaccttagggccaatccctaagtgtggcttcctaatccaggccagggatcattcagtgataccaccaggccaatcccagcattcctcctgcacaaagtgtttgtgtgtggggggatagattggggtggggccaccttcatttgaatcctgagtcattctaagagttctgcaagcttttgccttcagcaccctataccccctcgctctctgttccttctcaggttgacctttgtcccaatgcgggacagtccagaggcagatggggacctatgtgtgcctccaacctgcgttttcctcagaattcatcctgactccctctgacacagattgaggggggggggaagaaacccaacccgcaccaaagcaaaacactttgccttctaaaggctgtgcaccaagtagacgcagatggtcagcccacctttgtgtttccttaagatggaaattgtaactgatgctatttattgtttgtgtgtggtagcttgaagcacaccacggtccatgtgtttgtctgatacctatttcagcagcatttacaaaagccagtgtctggttacacttttcagttttcattaatcaacatgaaaatgttaccattggtgccagctgcaccctatttaatttttttaagggcactatatttgtacatttcgtttttaatgttaaagggcttcttaaagtttacagtacagttatcaagggaatagaggggatgcattagtgcctaaatgttattctagtggctgcaggcagcaacccagaagcagttttgaaaacaggttgtttccctctgtcctcccttatttgggaaaattcaagtgctttcttcacctttcaggcacctcacggtgactcccgttacttagagcagtctgtcgtcgtcttcttcttcttcctcttcctcttcctcttcctcttcctcttcctcttcctcttcctcttcctcttcctcttcttcttcttcttcttcttcttcttcttcttcttcttcctcctagggtttaaaatcccccttcctcttcccttatctcttaaactcttcatttgcactgggtcacacgtatgatttgtggttttaagaccaaagcaatgtcttattactcttctggagccgtttatgtgtactaaccaacccttccctccacttccctgggtttagatgcacacggttctcaaaggagcacaaacatggccagattcacagtgggacccacacagccatgttaaaaaaaaaaacaaaaaaaaacctcttcacttgtctgagaattttaacctgggccccaattgctaacgggcaccatggcttgggtttcaggttagggaacgttgcccagtgagtaacagaaagacttaactgatttaattagtttgccctcatccttcactccaagaccctaagaaaccgatcagagaaacatctccacaccattagaggttgagaagggaggagccatgtgggggttccctccgctcggaggaaaggcactgactgacctagttagagtggtagcagaagcacccatggtatctgagagagcaataaacaataaaagatgatcctcccaagcttagaatttgttgttcttaaagaagacgatgcatctaaaacaaagaatttttttttctatgtgggaactttcttcgttgtctgtttacttcatgtttacaaataattgtttgctttttaatgcagtactttaaaaaatatatcagccaaaaccataacttacagtaatttttttaggtattctgaataaaattccatgtcttttgatatgtctactgttcttaggtttctgtggaacaacaacaacaacaacaacaaaattgtagcattttgtgtaaatacagctttcgttcttattttttatttttctgattgctattgcccaagatttgctttctatgcacgtattgtacaaattgtgctttgtgccacaggtcatgatcgtggatgagtttactctgaacttcaaagggactatttgtattgtatgttgcaactgtaaattgaattatttggcatttccccctctcatgattgtaatattaatttgaagtttgaatttaattttcaataaaaaggctttttttttccttttggttttg

SEQ ID NO: 37 - DNA, 5904 bp, Mus musculus
  source 1..5904: mol_type = genomic DNA
atgcttcattcgcctcacaaacaaccacagaaccacaagtgcggtgcaaactttctccaggaagactgcaagaaggctctggcgtttaaatggttaatctctgcaggtcactaccagccaccgagaccaaccgagtcagtgagtgctctaaccacagtccatgcaggaatagtaggtccttcaaatatttgctcactccgttttgttttgtttccttgcttttcacatgttaccagctacataatttcttgacagaaaaaaataaatataaagtctatgtactccaggcatactgtacaactaaaacagggactgggtatggtttgtattttcagtttaaggctgcaagcagtatttacaacagagggcacaagttctatctggaaaaaaaaggagggactatggcgtcaaacagcctcttcagcgcagtgacaccgtgtcagcaaagcttcttttgggatccgagcaccagccggcgcttcagccccccctccagcagcctgcagcccggcaagatgagcgacgtgagcccggtggtggctgcgcagcagcagcaacagcagcagcagcagcagcaacagcagcagcaacaacagcaacagcaacaacagcagcagcagcagcagcagcaggaggcggccgcagcagcagcggcggcagcggcggcggcagcagcggcggcggccgcagtgccccgattgaggccgccgcacgacaaccgcaccatggtggagatcatcgcggaccacccggccgaactggtccgcaccgacagtcccaacttcctgtgctccgtgctgccctcgcactggcggtgcaacaagaccctgcccgtggccttcaaggttgtagccctcggagaggtaccagatgggactgtggttaccgtcatggccgggaatgatgagaactactccgccgagctccgaaatgcctccgctgttatgaaaaaccaagtagccaggttcaacgatctgagatttgtgggccggagcggacgaggcaagagtttcaccttgaccataacagtcttcacaaatcctccccaagtggccacttaccacagagctattaaagtgacagtggacggtccccgggaaccaagaaggcacagacagaagcttgatgactctaaacctagtttgttctctgatcgcctcagtgatttagggcgcattcctcatcccagtatgagagtaggtgtcccgcctcagaacccacggccctccctgaactctgcaccaagtccttttaatccacaaggacagagtcagattacagatcccaggcaggcacagtcttccccaccgtggtcctatgaccagtcttacccctcctatctgagccagatgacatccccatccatccactccaccacgccgctgtcttccacacggggcaccgggctacctgccatcactgacgtgcccaggcgtatttcagatgatgacactgccacctctgacttctgcctctggccttcctctctcagtaagaagagccaggcaggtgcttcagaactgggccctttttcagaccccaggcagttcccaagcatttcatccctcactgagagccgcttctccaacccacgaatgcactacccagccacctttacctacaccccgccagtcacgtcaggcatgtccctcggcatgtccgccaccactcactaccacacgtacctgccaccaccctaccccggctcttcccaaagccagagtggacccttccagaccagcagcactccatatctctactatggtacttcgtcagcatcctatcagttcccaatggtacccgggggagaccggtctccttccaggatggtcccaccatgcaccaccacctcgaatggcagcacgctattaaatccaaatttgcctaaccagaatgatggtgttgacgctgacggaagccacagcagttccccaactgttttgaattctagcggcagaatggatgagtctgtttggcggccatattgaaattcgtcaaccatggcccagtggcatgggggccacatcccgcatgtgttaatatatacatatataaagagagtgcctatatatgtatattgattagctaactagaagatttctcattcaatccctagtcatgatcttgcaaccctaagagggtgggggcagtcataactgggtttcatattgtttactatttaagatgtcccctttaccaaggaacaaaccgtcaaaggtgttgtctggtctgttttcataagtgacctgttcccacgccggttcagagaggtggactctgggtctgggaggaaggagagacacttcctctctgtgctttgaaaccacagcctctgctgtgtggcagccggtacactctgcagacccgcttacagagtcagatgtggtgcactcagaaagggacaagaggcagagtggctgcttctgtccgctgccgtccactctgccgtccacctgttccaaagttttccttcagacttgctgcaggtactcatttgaacttttgagttcactttttttttttcctattctaagaaagtgacttcaaaaatactgatcaggacagataattttattttaccttttatattttctcacttcccccatttaaccaaaaagaaatcccgttccccctcccccgttccttctgcttctccctttatgcaaactgaaaatggcaatgccttattattatagccataatggtatagtgtttgagttggctgtgtgttatgtgtttttttctttttttttcttttttaaattatgaatatgtgtaaaatctgaagtaacttgctaacgtgaatggtcatataactttaaagatatatttataattatttaatgacatttggacatttggaacatttcttagtgtaatgatatgttgacttcggtctctaaaagtgtgcttcttcttcaataccaagtttcttcagtgggctagagccatatcggaaatattgctaagcaatctcaattccttcaggcataatgtgatttttttttttttttgaagataactcccatctccaaatagtttagatgtagtttgttttcacgatgtatgaaggagatgctctgtttctttctttcaggcatttgattgcctctgacacagctttgccttttaaagcaataattagggattaaaataacaaaaacaaaacaaaagccacctatagccctttaacacttaacgtggcccctttactagcatgaaatgctggagacatgtggtttcctaatttctccattttgggggtggtgggaggggggagggtggccattatgactcttatcatattaaaaagccaagcacaagtgattggttgaactgcagaaaagtgttctgtggtctctgagttgagcaaaactctaaattgcaggcttcgtggttgagggcctagtcagctgaaagccacgcgtgtggtaaaggctcaggcatggcttggagaacctaggaacacattaggagcctgcacctaccagcctcaccatacagccattcaggggaacccaaaaagtgccttacccaaggagggcccccagcagctttccaggaagtcgaatgaagtcgctgtcctcggggaactggtcagctgaagtagcaaccggtagctgatgtcagtagacaacagaacctgtggggacctccaggaaacctttgacattggaggctttcattaggcagggccaacaagagcagggaaggccatgtacccattggtatctgccattgtgtgtgagtttgagatccagccctccttggaggatgtactgtgatcattcctggtaccttagggccaatccctaagtgtggcttcctaatccaggccagggatcattcagtgataccaccaggccaatcccagcattcctcctgcacaaagtgtttgtgtgtggggggatagattggggtggggccaccttcatttgaatcctgagtcattctaagagttctgcaagcttttgccttcagcaccctataccccctcgctctctgttccttctcaggttgacctttgtcccaatgcgggacagtccagaggcagatggggacctatgtgtgcctccaacctgcgttttcctcagaattcatcctgactccctctgacacagattgaggggggggggaagaaacccaacccgcaccaaagcaaaacactttgccttctaaaggctgtgcaccaagtagacgcagatggtcagcccacctttgtgtttccttaagatggaaattgtaactgatgctatttattgtttgtgtgtggtagcttgaagcacaccacggtccatgtgtttgtctgatacctatttcagcagcatttacaaaagccagtgtctggttacacttttcagttttcattaatcaacatgaaaatgttaccattggtgccagctgcaccctatttaatttttttaagggcactatatttgtacatttcgtttttaatgttaaagggcttcttaaagtttacagtacagttatcaagggaatagaggggatgcattagtgcctaaatgttattctagtggctgcaggcagcaacccagaagcagttttgaaaacaggttgtttccctctgtcctcccttatttgggaaaattcaagtgctttcttcacctttcaggcacctcacggtgactcccgttacttagagcagtctgtcgtcgtcttcttcttcttcctcttcctcttcctcttcctcttcctcttcctcttcctcttcctcttcctcttcctcttcttcttcttcttcttcttcttcttcttcttcttcttcctcctagggtttaaaatcccccttcctcttcccttatctcttaaactcttcatttgcactgggtcacacgtatgatttgtggttttaagaccaaagcaatgtcttattactcttctggagccgtttatgtgtactaaccaacccttccctccacttccctgggtttagatgcacacggttctcaaaggagcacaaacatggccagattcacagtgggacccacacagccatgttaaaaaaaaaaacaaaaaaaaacctcttcacttgtctgagaattttaacctgggccccaattgctaacgggcaccatggcttgggtttcaggttagggaacgttgcccagtgagtaacagaaagacttaactgatttaattagtttgccctcatccttcactccaagaccctaagaaaccgatcagagaaacatctccacaccattagaggttgagaagggaggagccatgtgggggttccctccgctcggaggaaaggcactgactgacctagttagagtggtagcagaagcacccatggtatctgagagagcaataaacaataaaagatgatcctcccaagcttagaatttgttgttcttaaagaagacgatgcatctaaaacaaagaatttttttttctatgtgggaactttcttcgttgtctgtttacttcatgtttacaaataattgtttgctttttaatgcagtactttaaaaaatatatcagccaaaaccataacttacagtaatttttttaggtattctgaataaaattccatgtcttttgatatgtctactgttcttaggtttctgtggaacaacaacaacaacaacaacaaaattgtagcattttgtgtaaatacagctttcgttcttattttttatttttctgattgctattgcccaagatttgctttctatgcacgtattgtacaaattgtgctttgtgccacaggtcatgatcgtggatgagtttactctgaacttcaaagggactatttgtattgtatgttgcaactgtaaattgaattatttggcatttccccctctcatgattgtaatattaatttgaagtttgaatttaattttcaataaaaaggctttttttttccttttggttttg

SEQ ID NO: 38 - DNA, 5829 bp, Mus musculus
  source 1..5829: mol_type = genomic DNA
ggcggaggaggcggcgcgggcaagcgacggcgccgcgagctgggcagccgcgctctgcttggcggtggcggacagcgaggagccacacgcaccgccgagatggactgctgaacctgcggggctccactaccgtactggaacccggagcggggcgccagcgcacccaggacacggtgccccaaggggcccctactgcaagctgttaacttcaagtcccttgcggcggctgggccaaacacgcccccgcgtcccgctcgttgcagccaccgccagggactcccaagcctcccggtggagatccgcgcctctcgggtgtccccacccgctaccccgactctgtccggtctccagtcgccggcccccagcagactccgcccggcacacggcttcctcgaacttgatttctcacctcctctgtcccgtcacctccatcctctttccgcccccgtctcgcctccaccccctcgatttcctcctcctcgccccccatttccaccctcctccccctcccccggccacttcgctaacttgtggctgttgtgatgcgtattcctgtagatccgagcaccagccggcgcttcagccccccctccagcagcctgcagcccggcaagatgagcgacgtgagcccggtggtggctgcgcagcagcagcaacagcagcagcagcagcagcaacagcagcagcaacaacagcaacagcaacaacagcagcagcagcagcagcagcaggaggcggccgcagcagcagcggcggcagcggcggcggcagcagcggcggcggccgcagtgccccgattgaggccgccgcacgacaaccgcaccatggtggagatcatcgcggaccacccggccgaactggtccgcaccgacagtcccaacttcctgtgctccgtgctgccctcgcactggcggtgcaacaagaccctgcccgtggccttcaaggttgtagccctcggagaggtaccagatgggactgtggttaccgtcatggccgggaatgatgagaactactccgccgagctccgaaatgcctccgctgttatgaaaaaccaagtagccaggttcaacgatctgagatttgtgggccggagcggacgaggcaagagtttcaccttgaccataacagtcttcacaaatcctccccaagtggccacttaccacagagctattaaagtgacagtggacggtccccgggaaccaagaaatcccaggcaggcacagtcttccccaccgtggtcctatgaccagtcttacccctcctatctgagccagatgacatccccatccatccactccaccacgccgctgtcttccacacggggcaccgggctacctgccatcactgacgtgcccaggcgtatttcagatgatgacactgccacctctgacttctgcctctggccttcctctctcagtaagaagagccaggcaggtgcttcagaactgggccctttttcagaccccaggcagttcccaagcatttcatccctcactgagagccgcttctccaacccacgaatgcactacccagccacctttacctacaccccgccagtcacgtcaggcatgtccctcggcatgtccgccaccactcactaccacacgtacctgccaccaccctaccccggctcttcccaaagccagagtggacccttccagaccagcagcactccatatctctactatggtacttcgtcagcatcctatcagttcccaatggtacccgggggagaccggtctccttccaggatggtcccaccatgcaccaccacctcgaatggcagcacgctattaaatccaaatttgcctaaccagaatgatggtgttgacgctgacggaagccacagcagttccccaactgttttgaattctagcggcagaatggatgagtctgtttggcggccatattgaaattcgtcaaccatggcccagtggcatgggggccacatcccgcatgtgttaatatatacatatataaagagagtgcctatatatgtatattgattagctaactagaagatttctcattcaatccctagtcatgatcttgcaaccctaagagggtgggggcagtcataactgggtttcatattgtttactatttaagatgtcccctttaccaaggaacaaaccgtcaaaggtgttgtctggtctgttttcataagtgacctgttcccacgccggttcagagaggtggactctgggtctgggaggaaggagagacacttcctctctgtgctttgaaaccacagcctctgctgtgtggcagccggtacactctgcagacccgcttacagagtcagatgtggtgcactcagaaagggacaagaggcagagtggctgcttctgtccgctgccgtccactctgccgtccacctgttccaaagttttccttcagacttgctgcaggtactcatttgaacttttgagttcactttttttttttcctattctaagaaagtgacttcaaaaatactgatcaggacagataattttattttaccttttatattttctcacttcccccatttaaccaaaaagaaatcccgttccccctcccccgttccttctgcttctccctttatgcaaactgaaaatggcaatgccttattattatagccataatggtatagtgtttgagttggctgtgtgttatgtgtttttttctttttttttcttttttaaattatgaatatgtgtaaaatctgaagtaacttgctaacgtgaatggtcatataactttaaagatatatttataattatttaatgacatttggacatttggaacatttcttagtgtaatgatatgttgacttcggtctctaaaagtgtgcttcttcttcaataccaagtttcttcagtgggctagagccatatcggaaatattgctaagcaatctcaattccttcaggcataatgtgatttttttttttttttgaagataactcccatctccaaatagtttagatgtagtttgttttcacgatgtatgaaggagatgctctgtttctttctttcaggcatttgattgcctctgacacagctttgccttttaaagcaataattagggattaaaataacaaaaacaaaacaaaagccacctatagccctttaacacttaacgtggcccctttactagcatgaaatgctggagacatgtggtttcctaatttctccattttgggggtggtgggaggggggagggtggccattatgactcttatcatattaaaaagccaagcacaagtgattggttgaactgcagaaaagtgttctgtggtctctgagttgagcaaaactctaaattgcaggcttcgtggttgagggcctagtcagctgaaagccacgcgtgtggtaaaggctcaggcatggcttggagaacctaggaacacattaggagcctgcacctaccagcctcaccatacagccattcaggggaacccaaaaagtgccttacccaaggagggcccccagcagctttccaggaagtcgaatgaagtcgctgtcctcggggaactggtcagctgaagtagcaaccggtagctgatgtcagtagacaacagaacctgtggggacctccaggaaacctttgacattggaggctttcattaggcagggccaacaagagcagggaaggccatgtacccattggtatctgccattgtgtgtgagtttgagatccagccctccttggaggatgtactgtgatcattcctggtaccttagggccaatccctaagtgtggcttcctaatccaggccagggatcattcagtgataccaccaggccaatcccagcattcctcctgcacaaagtgtttgtgtgtggggggatagattggggtggggccaccttcatttgaatcctgagtcattctaagagttctgcaagcttttgccttcagcaccctataccccctcgctctctgttccttctcaggttgacctttgtcccaatgcgggacagtccagaggcagatggggacctatgtgtgcctccaacctgcgttttcctcagaattcatcctgactccctctgacacagattgaggggggggggaagaaacccaacccgcaccaaagcaaaacactttgccttctaaaggctgtgcaccaagtagacgcagatggtcagcccacctttgtgtttccttaagatggaaattgtaactgatgctatttattgtttgtgtgtggtagcttgaagcacaccacggtccatgtgtttgtctgatacctatttcagcagcatttacaaaagccagtgtctggttacacttttcagttttcattaatcaacatgaaaatgttaccattggtgccagctgcaccctatttaatttttttaagggcactatatttgtacatttcgtttttaatgttaaagggcttcttaaagtttacagtacagttatcaagggaatagaggggatgcattagtgcctaaatgttattctagtggctgcaggcagcaacccagaagcagttttgaaaacaggttgtttccctctgtcctcccttatttgggaaaattcaagtgctttcttcacctttcaggcacctcacggtgactcccgttacttagagcagtctgtcgtcgtcttcttcttcttcctcttcctcttcctcttcctcttcctcttcctcttcctcttcctcttcctcttcctcttcttcttcttcttcttcttcttcttcttcttcttcttcctcctagggtttaaaatcccccttcctcttcccttatctcttaaactcttcatttgcactgggtcacacgtatgatttgtggttttaagaccaaagcaatgtcttattactcttctggagccgtttatgtgtactaaccaacccttccctccacttccctgggtttagatgcacacggttctcaaaggagcacaaacatggccagattcacagtgggacccacacagccatgttaaaaaaaaaaacaaaaaaaaacctcttcacttgtctgagaattttaacctgggccccaattgctaacgggcaccatggcttgggtttcaggttagggaacgttgcccagtgagtaacagaaagacttaactgatttaattagtttgccctcatccttcactccaagaccctaagaaaccgatcagagaaacatctccacaccattagaggttgagaagggaggagccatgtgggggttccctccgctcggaggaaaggcactgactgacctagttagagtggtagcagaagcacccatggtatctgagagagcaataaacaataaaagatgatcctcccaagcttagaatttgttgttcttaaagaagacgatgcatctaaaacaaagaatttttttttctatgtgggaactttcttcgttgtctgtttacttcatgtttacaaataattgtttgctttttaatgcagtactttaaaaaatatatcagccaaaaccataacttacagtaatttttttaggtattctgaataaaattccatgtcttttgatatgtctactgttcttaggtttctgtggaacaacaacaacaacaacaacaaaattgtagcattttgtgtaaatacagctttcgttcttattttttatttttctgattgctattgcccaagatttgctttctatgcacgtattgtacaaattgtgctttgtgccacaggtcatgatcgtggatgagtttactctgaacttcaaagggactatttgtattgtatgttgcaactgtaaattgaattatttggcatttccccctctcatgattgtaatattaatttgaagtttgaatttaattttcaataaaaaggctttttttttccttttggttttg

SEQ ID NO: 39 - DNA, 5724 bp, Mus musculus
  source 1..5724: mol_type = genomic DNA
ggcggaggaggcggcgcgggcaagcgacggcgccgcgagctgggcagccgcgctctgcttggcggtggcggacagcgaggagccacacgcaccgccgagatggactgctgaacctgcggggctccactaccgtactggaacccggagcggggcgccagcgcacccaggacacggtgccccaaggggcccctactgcaagctgttaacttcaagtcccttgcggcggctgggccaaacacgcccccgcgtcccgctcgttgcagccaccgccagggactcccaagcctcccggtggagatccgcgcctctcgggtgtccccacccgctaccccgactctgtccggtctccagtcgccggcccccagcagactccgcccggcacacggcttcctcgaacttgatttctcacctcctctgtcccgtcacctccatcctctttccgcccccgtctcgcctccaccccctcgatttcctcctcctcgccccccatttccaccctcctccccctcccccggccacttcgctaacttgtggctgttgtgatgcgtattcctgtagatccgagcaccagccggcgcttcagccccccctccagcagcctgcagcccggcaagatgagcgacgtgagcccggtggtggctgcgcagcagcagcaacagcagcagcagcagcagcaacagcagcagcaacaacagcaacagcaacaacagcagcagcagcagcagcagcaggaggcggccgcagcagcagcggcggcagcggcggcggcagcagcggcggcggccgcagtgccccgattgaggccgccgcacgacaaccgcaccatggtggagatcatcgcggaccacccggccgaactggtccgcaccgacagtcccaacttcctgtgctccgtgctgccctcgcactggcggtgcaacaagaccctgcccgtggccttcaaggttgtagccctcggagaggtaccagatgggactgtggttaccgtcatggccgggaatgatgagaactactccgccgagctccgaaatgcctccgctgttatgaaaaaccaagtagccaggttcaacgatctgagatttgtgggccggagcggacgagatcccaggcaggcacagtcttccccaccgtggtcctatgaccagtcttacccctcctatctgagccagatgacatccccatccatccactccaccacgccgctgtcttccacacggggcaccgggctacctgccatcactgacgtgcccaggcgtatttcagatgatgacactgccacctctgacttctgcctctggccttcctctctcagtaagaagagccaggcaggtgcttcagaactgggccctttttcagaccccaggcagttcccaagcatttcatccctcactgagagccgcttctccaacccacgaatgcactacccagccacctttacctacaccccgccagtcacgtcaggcatgtccctcggcatgtccgccaccactcactaccacacgtacctgccaccaccctaccccggctcttcccaaagccagagtggacccttccagaccagcagcactccatatctctactatggtacttcgtcagcatcctatcagttcccaatggtacccgggggagaccggtctccttccaggatggtcccaccatgcaccaccacctcgaatggcagcacgctattaaatccaaatttgcctaaccagaatgatggtgttgacgctgacggaagccacagcagttccccaactgttttgaattctagcggcagaatggatgagtctgtttggcggccatattgaaattcgtcaaccatggcccagtggcatgggggccacatcccgcatgtgttaatatatacatatataaagagagtgcctatatatgtatattgattagctaactagaagatttctcattcaatccctagtcatgatcttgcaaccctaagagggtgggggcagtcataactgggtttcatattgtttactatttaagatgtcccctttaccaaggaacaaaccgtcaaaggtgttgtctggtctgttttcataagtgacctgttcccacgccggttcagagaggtggactctgggtctgggaggaaggagagacacttcctctctgtgctttgaaaccacagcctctgctgtgtggcagccggtacactctgcagacccgcttacagagtcagatgtggtgcactcagaaagggacaagaggcagagtggctgcttctgtccgctgccgtccactctgccgtccacctgttccaaagttttccttcagacttgctgcaggtactcatttgaacttttgagttcactttttttttttcctattctaagaaagtgacttcaaaaatactgatcaggacagataattttattttaccttttatattttctcacttcccccatttaaccaaaaagaaatcccgttccccctcccccgttccttctgcttctccctttatgcaaactgaaaatggcaatgccttattattatagccataatggtatagtgtttgagttggctgtgtgttatgtgtttttttctttttttttcttttttaaattatgaatatgtgtaaaatctgaagtaacttgctaacgtgaatggtcatataactttaaagatatatttataattatttaatgacatttggacatttggaacatttcttagtgtaatgatatgttgacttcggtctctaaaagtgtgcttcttcttcaataccaagtttcttcagtgggctagagccatatcggaaatattgctaagcaatctcaattccttcaggcataatgtgatttttttttttttttgaagataactcccatctccaaatagtttagatgtagtttgttttcacgatgtatgaaggagatgctctgtttctttctttcaggcatttgattgcctctgacacagctttgccttttaaagcaataattagggattaaaataacaaaaacaaaacaaaagccacctatagccctttaacacttaacgtggcccctttactagcatgaaatgctggagacatgtggtttcctaatttctccattttgggggtggtgggaggggggagggtggccattatgactcttatcatattaaaaagccaagcacaagtgattggttgaactgcagaaaagtgttctgtggtctctgagttgagcaaaactctaaattgcaggcttcgtggttgagggcctagtcagctgaaagccacgcgtgtggtaaaggctcaggcatggcttggagaacctaggaacacattaggagcctgcacctaccagcctcaccatacagccattcaggggaacccaaaaagtgccttacccaaggagggcccccagcagctttccaggaagtcgaatgaagtcgctgtcctcggggaactggtcagctgaagtagcaaccggtagctgatgtcagtagacaacagaacctgtggggacctccaggaaacctttgacattggaggctttcattaggcagggccaacaagagcagggaaggccatgtacccattggtatctgccattgtgtgtgagtttgagatccagccctccttggaggatgtactgtgatcattcctggtaccttagggccaatccctaagtgtggcttcctaatccaggccagggatcattcagtgataccaccaggccaatcccagcattcctcctgcacaaagtgtttgtgtgtggggggatagattggggtggggccaccttcatttgaatcctgagtcattctaagagttctgcaagcttttgccttcagcaccctataccccctcgctctctgttccttctcaggttgacctttgtcccaatgcgggacagtccagaggcagatggggacctatgtgtgcctccaacctgcgttttcctcagaattcatcctgactccctctgacacagattgaggggggggggaagaaacccaacccgcaccaaagcaaaacactttgccttctaaaggctgtgcaccaagtagacgcagatggtcagcccacctttgtgtttccttaagatggaaattgtaactgatgctatttattgtttgtgtgtggtagcttgaagcacaccacggtccatgtgtttgtctgatacctatttcagcagcatttacaaaagccagtgtctggttacacttttcagttttcattaatcaacatgaaaatgttaccattggtgccagctgcaccctatttaatttttttaagggcactatatttgtacatttcgtttttaatgttaaagggcttcttaaagtttacagtacagttatcaagggaatagaggggatgcattagtgcctaaatgttattctagtggctgcaggcagcaacccagaagcagttttgaaaacaggttgtttccctctgtcctcccttatttgggaaaattcaagtgctttcttcacctttcaggcacctcacggtgactcccgttacttagagcagtctgtcgtcgtcttcttcttcttcctcttcctcttcctcttcctcttcctcttcctcttcctcttcctcttcctcttcctcttcttcttcttcttcttcttcttcttcttcttcttcttcctcctagggtttaaaatcccccttcctcttcccttatctcttaaactcttcatttgcactgggtcacacgtatgatttgtggttttaagaccaaagcaatgtcttattactcttctggagccgtttatgtgtactaaccaacccttccctccacttccctgggtttagatgcacacggttctcaaaggagcacaaacatggccagattcacagtgggacccacacagccatgttaaaaaaaaaaacaaaaaaaaacctcttcacttgtctgagaattttaacctgggccccaattgctaacgggcaccatggcttgggtttcaggttagggaacgttgcccagtgagtaacagaaagacttaactgatttaattagtttgccctcatccttcactccaagaccctaagaaaccgatcagagaaacatctccacaccattagaggttgagaagggaggagccatgtgggggttccctccgctcggaggaaaggcactgactgacctagttagagtggtagcagaagcacccatggtatctgagagagcaataaacaataaaagatgatcctcccaagcttagaatttgttgttcttaaagaagacgatgcatctaaaacaaagaatttttttttctatgtgggaactttcttcgttgtctgtttacttcatgtttacaaataattgtttgctttttaatgcagtactttaaaaaatatatcagccaaaaccataacttacagtaatttttttaggtattctgaataaaattccatgtcttttgatatgtctactgttcttaggtttctgtggaacaacaacaacaacaacaacaaaattgtagcattttgtgtaaatacagctttcgttcttattttttatttttctgattgctattgcccaagatttgctttctatgcacgtattgtacaaattgtgctttgtgccacaggtcatgatcgtggatgagtttactctgaacttcaaagggactatttgtattgtatgttgcaactgtaaattgaattatttggcatttccccctctcatgattgtaatattaatttgaagtttgaatttaattttcaataaaaaggctttttttttccttttggttttg